(19) United States
(12) Patent Application Publication
(10) Pub. No.: US 2007/0078144 A1
(43) Pub. Date: Apr. 5, 2007

(54) Agents for treating neurodegenerative diseases

(76) Inventors: Brent R. Stockwell, New York, NY (US); Benjamin Hoffstrom, New York, NY (US); Hemant Varma, New York, NY (US)

Correspondence Address: BAKER & BOTTS L.L.P., 30 ROCKEFELLER PLAZA, 44TH FLOOR, NEW YORK, NY, 10112-4498, US

(21) Appl. No.: 11/498,110

(22) Filed: Aug. 2, 2006

Related U.S. Application Data
(63) Continuation-in-part of application No. 11/349,653, filed on Feb. 7, 2006.
Application No. 11/349,653 is a continuation-in-part of application No. 10/837,360, filed on Apr. 30, 2004.
Application No. 11/349,653 is a continuation-in-part of application No. 10/767,591, filed on Jan. 29, 2004, now abandoned.
(60) Provisional application No. 60/467,290, filed on May 2, 2003.
(60) Provisional application No. 60/496,209, filed on Aug. 19, 2003.
(60) Provisional application No. 60/482,688, filed on Jun. 25, 2003.
(60) Provisional application No. 60/467,290, filed on May 2, 2003.
(60) Provisional application No. 60/457,401, filed on Mar. 25, 2003.
(60) Provisional application No. 60/443,728, filed on Jan. 29, 2003.

(51) Int. Cl. A61K 31/496; A61K 31/4709; A61K 31/452; A61K 31/4025

(52) U.S. Cl. 514/254.11; 514/307; 514/422; 514/320; 514/314

(57) ABSTRACT

The present invention relates to compounds effective in preventing neuronal cell death, which may be used in the treatment of neurodegenerative diseases. It is based, at least in part, on the discovery that particular compounds were effective in preventing neuronal death in model systems of Huntington's Disease.

### RELATED APPLICATIONS

This application is a continuation-in-part of U.S. patent application Ser. No. 11/349,653, filed Feb. 7, 2006, which is a continuation-in-part of U.S. patent application Ser. No. 10/837,360, filed Apr. 30, 2004, which claims the benefit of U.S. Provisional Application No. 60/467,290, filed May 2, 2003, and is a Continuation-in-part of U.S. application Ser. No. 10/767,591, filed Jan. 29, 2004, which claims the benefit of U.S. Provisional Application No. 60/496,209, filed Aug. 19, 2003; U.S. Provisional Application No. 60/482,688, filed Jun. 25, 2003; U.S. Provisional Application No. 60/467,290, filed May 2, 2003; U.S. Provisional Application No. 60/457,401, filed Mar. 25, 2003; and U.S. Provisional Application No. 60/443,728, filed Jan. 29, 2003. The teachings of these referenced Applications are incorporated herein by reference in their entireties.

Work described herein was funded, in whole or in part, by National Cancer Institute Grant 1R01CA97061-01. The United States government has certain rights in the invention.

### BACKGROUND OF THE INVENTION

Huntington's Disease (HD) is one of nine inherited neurodegenerative disorders caused by trinucleotide (CAG) repeat expansion. Huntington's disease (HD) is a fatal autosomal dominant neurodegenerative disease, characterized by selective neuronal loss in the striatum and cortex. (Tobin, A. J. & Signer, E. R. Huntington's disease: the challenge for cell biologists. Trends Cell Biol 10, 531-536 (2000)). There are nine inherited neurodegenerative disorders caused by a polyglutamine (polyQ)-encoding trinucleotide (CAG) repeat expansion within the coding sequence of a gene. These diseases include HD, Spinobulbar muscular atrophy (SBMA), dentatorubral pallidoluysian atrophy (DRPLA), and the spinocerebellar ataxias type 1, 2, 3, 6, 7, and 17. Although the length of the CAG expansion is variable in the different disorders, it appears that the threshold for toxicity is approximately 40 CAG repeats, with longer repeat lengths generally resulting in earlier disease onset. Precisely how polyQ mutations lead to neuronal loss in each disease remains unclear; however, several molecular characteristics appear to be shared among the different disorders. Mutant htt has been proposed to cause toxicity by multiple mechanisms, including protein aggregation, transcriptional dysregulation, mitochondrial dysfunction, altered intracellular transport and activation of apoptotic machinery. (Ross, C. A. Polyglutamine pathogenesis: emergence of unifying mechanisms for Huntington's disease and related disorders. Neuron 35, 819-822 (2002)).

HD has a complex phenotype involving neuronal dysfunction and death that occurs over decades in HD patients. This precludes an exact recapitulation of the human disease phenotype in cell culture models that would be amenable to rapid compound screening. However, models that recapitulate some aspects of HD have been developed. The phenotypes in such models range from aggregation of mutant htt, specific cellular dysfunctions (in some cases susceptibility to stresses) and cell death. (Sipione, S. & Cattaneo, E. Modeling Huntington's disease in cells, flies, and mice. Mol Neurobiol 23, 21-51 (2001)). Mechanism-based screens that seek to reverse specific phenotypes, such as aggregation of mutant htt, have identified leads. (Heiser, V. et al. Identification of benzothiazoles as potential polyglutamine aggregation inhibitors of Huntington's disease by using an automated filter retardation assay. Proceedings of the National Academy of Sciences of the United States of America 99 Suppl 4, 16400-16406 (2002); Zhang, X. et al. A potent small molecule inhibits polyglutamine aggregation in Huntington's disease neurons and suppresses neurodegeneration in vivo. Proc Natl Acad Sci USA 102, 892-897 (2005)). However, the role of aggregates in HD pathology is unresolved, and they may be protective. (Arrasate, M., Mitra, S., Schweitzer, E. S., Segal, M. R. & Finkbeiner, S. Inclusion body formation reduces levels of mutant huntingtin and the risk of neuronal death. Nature 431, 805-810 (2004)). Assays that seek to reverse a toxic phenotype induced by mutant htt (cell death, dysfunction or cell vulnerability to stresses) without assumptions of the underlying mechanisms may provide a wider range of mechanistic interventions. (Aiken, C. T., Tobin, A. J. & Schweitzer, E. S. A cell-based screen for drugs to treat Huntington's disease. Neurobiol Dis 16, 546-555 (2004)).

Striatal neuronal cell loss is ubiquitous in HD patients. (Hickey, M. A. & Chesselet, M. F. Apoptosis in Huntington's disease. Prog Neuropsychopharmacol Biol Psychiatry 27, 255-265 (2003)). Though the role for cell death in HD pathophysiology is debated, evidence points to prevention of neuronal death as a valid therapeutic end point. Pharmacological and genetic prevention of neuronal death alleviates disease symptoms and extends life-span in a transgenic HD mouse model. (Chen, M. et al. Minocycline inhibits caspase-1 and caspase-3 expression and delays mortality in a transgenic mouse model of Huntington disease. [see comment]. Nature Medicine 6, 797-801 (2000); Ona, V. O. et al. Inhibition of caspase-1 slows disease progression in a mouse model of Huntington's disease. Nature 399, 263-267 (1999)). Additionally, neuronal transplantation, to replace lost neuronal cell functions, may have a role in HD therapy. (Bachoud-Lâevi, A. C. et al. Motor and cognitive improvements in patients with Huntington's disease after neural transplantation. Lancet 356, 1975-1979 (2000)). Thus, identification of cellular pathways that prevent death of mutant-htt-expressing neurons and small molecules that target these pathways have therapeutic potential.

Therefore, there is a need to develop novel methods and compounds for treating HD.

### SUMMARY OF THE INVENTION

The present invention provides for compounds which may be used to inhibit neuronal cell death, for example in the context of neurodegenerative disorders such as Huntington's disease (HD). The present invention further provides for a genotype-selective method for identifying additional drugs or agents for treating or preventing neurodegenerative disorders.

In certain embodiments, the invention relates to isolated compounds or their analogs that suppress neuronal cell toxicity caused by polyQ expansion. For example, the compounds of the invention may be encompassed within a general formula as set forth in Formulas I-XIV herein, or have a specific formula as shown in FIG. 6, 16, 17, 19, 20, 36, or 37. In further embodiments, the invention provides analogs of the subject compounds in FIGS. 6, 16-17, 19-20, and 36-37, wherein the analogs selectively suppress neuronal cell toxicity caused by polyQ expansion. The compounds and analogs of the invention may be formulated with a pharmaceutically acceptable carrier, in amounts effective at inhibiting neuronal cell death and/or neuronal degeneration, as pharmaceutical compositions.

In further non-limiting embodiments, the present invention relates to a method of treating or preventing a neurodegenerative disorder associated with polyglutamine (polyQ) expansion in an individual comprising administering to the individual in need of the treatment, a therapeutically effective amount of a compound identified by the methods, such as a tubulin inhibitor (e.g., as shown in FIG. 5), other known compounds (e.g., see FIG. 24), a compound of Formula I-XIV (as set forth below) or a compound shown in FIG. 6, 16, 17, 18, 19, 20, 36, or 37, or an analog thereof. Examples of the neurodegenerative disorders which may be treated include, but are not limited to, Huntington's disease, spinobulbar muscular atrophy, dentatorubral pallidoluysian atrophy, and the spinocerebellar ataxias type 1, 2, 3, 6, 7, and 17.

In other certain non-limiting embodiments, the present invention relates to screening methods for identifying compounds that suppress neuronal cell toxicity caused by polyglutamine (polyQ) expansion. For example, the engineered neuronal cells express a polyQ-expanded protein which causes toxicity. Preferably, the identified compounds suppress such toxicity to engineered neuronal cells, but not their isogenic normal cell counterparts. An example of engineered neuronal cells includes engineered neuronal cells expressing a mutant huntingtin protein. The method has been used to identify known and novel compounds which protect against the neurotoxic effects of the huntingtin protein (e.g., see FIGS. 5-6, 16-20, 24, 36, and 37). For example, these compounds include known tubulin inhibitors (e.g., colchicines, podophyllotoxin, vincristine, and vinblastine), thiomuscimol, N-P-tosyl-L-valine chloromethyl ketone, parthenolide, forskolin, 1-methylisoguanosine, dihydrocytocholasin-B, 2-phenyaminoadenosine, and compounds as set forth in FIGS. 16-20 and 36-37. In certain cases, these compounds have increased toxicity-suppressing activity in the presence of a mutant huntingtin protein.

In certain embodiments, the invention relates to a method of identifying agents (drugs) that selectively suppress neuronal cell toxicity or selectively promote viability or growth of neuronal cells, such as engineered neuronal cells. For example, the neuronal cells are engineered to express a polyQ-expanded protein which causes toxicity. Preferably, the engineered neuronal cells are engineered human neuronal cells. In one embodiment, the invention provides a method of identifying an agent (drug) that selectively suppresses neuronal cell toxicity or promotes neuronal cell viability in engineered mammalian neuronal cells, comprising contacting test cells (e.g., engineered human neuronal cells), with a candidate agent; determining viability of test cells contacted with the candidate agent; and comparing the viability of the test cells with the viability of an appropriate control. In all embodiments, viability is assessed by determining the ability of an agent (drug) to suppress toxicity or to promote growth/proliferation of cells, or both. If the viability of the test cells is more than that of the control cells, then an agent (drug) that selectively suppresses neuronal cell toxicity (or promotes neuronal cell growth) is identified. An appropriate control is a cell that is the same type of cell as that of test cells except that the control cell is not engineered to express a polyQ-expanded protein which causes toxicity. For example, control cells may be the parental primary cells from which the test cells are derived. Control cells are contacted with the candidate agent under the same conditions as the test cells. An appropriate control may be run simultaneously, or it may be pre-established (e.g., a pre-established standard or reference).

In one embodiment, the method identifies an agent that selectively suppresses neuronal cell toxicity. Such method comprises further assessing the selective toxicity-suppressing activity of an agent identified as a result of screening in engineered neuronal cells in an appropriate animal model or in an additional cell-based or non cell-based system or assay. For example, an agent or drug so identified can be assessed for its toxicity-suppressing activity in neuronal cells obtained from individuals suffering from or at risk of having HD. The method can further assess the selective toxicity-suppressing activity of an agent (drug) in an appropriate mouse model or nonhuman primate. The invention further relates to a method of identifying and producing an agent (drug), such as an agent (drug) that selectively suppresses toxicity to engineered neuronal cells.

In certain embodiments of the invention, a candidate agent is identified by screening an annotated compound library, a combinatorial library, or other library which comprises unknown or known compounds (agents, drugs) or both.

In certain embodiments, the invention relates to methods of identifying cellular components involved in polyglutamine-mediated neurotoxicity. Cellular components include, for example, proteins (e.g., enzymes, receptors), nucleic acids (e.g., DNA, RNA), and lipids (e.g., phospholipids). In one embodiment, the invention provides a method of identifying at least one (one or more) cellular component involved in polyglutamine-mediated neurotoxicity. This method comprises the following steps: (a) a cell, such as an engineered neuronal cell, is contacted with an identified subject compound (known or novel) that selectively suppresses toxicity to neuronal cells (e.g., a tubulin inhibitor (see FIG. 5) and a compound shown in FIGS. 6 and 16-20, or 36-37, or an analog thereof); and (b) a cellular component that interacts with the subject compound, either directly or indirectly, is identified. The cellular component that is identified is a cellular component involved in polyglutamine-mediated neurotoxicity.

In certain embodiments, the invention relates to a method of identifying agents (drugs) that interact with a (one or more) cellular component that interacts, directly or indirectly, with an identified compound that selectively suppresses toxicity to neuronal cells. This method comprises: (a) contacting a cell with a subject compound of the invention; (b) identifying a cellular component that interacts (directly or indirectly) with the compound; (c) contacting a cell with a candidate agent, which is an agent or drug to be assessed for its ability to interact with the identified cellular component(s); and (d) determining whether the agent interacts (directly or indirectly) with the cellular component in (b). If the agent interacts with the cellular component in (b), it is an agent that interacts with a cellular component interacting with a subject compound of the invention. In certain embodiments, the cell is an engineered neuronal cell such as a neuronal cell over-expressing a mutant huntingtin protein. In certain embodiments, the cellular component that interacts with a subject compound is involved in polyglutamine-mediated neurotoxicity. The identified cellular component (e.g., a protein or a nucleic acid) and an agent (drug) that is shown to interact with the identified cellular component can be synthesized using known methods.

In certain embodiments, the present invention relates to methods of conducting a drug discovery business. In one embodiment, such methods comprise: (a) identifying an (one or more) agent (drug) that selectively suppresses toxicity to neuronal cells; (b) assessing the efficacy and toxicity of the agent identified in (a), or analogs thereof, in animals; and (c) formulating a pharmaceutical preparation including one or more agents assessed in (b). For example, the identified agent is a known compound (e.g., see FIG. 24), a tubulin inhibitor, or a compound shown in FIGS. 6, 16-20, 36-37, or an analog thereof. In other embodiments, these methods of the invention contemplate compounds (e.g., cellular components) that interact with the subject agents, or compounds that interact with the identified cellular component as described above. The efficacy assessed may be the ability of an agent to selectively suppress toxicity to or promote viability of cells in an animal. In a further embodiment, these methods comprise establishing a distribution system for distributing the pharmaceutical preparation for sale. Optionally, a sales group is established for marketing the pharmaceutical preparation.

In further embodiments, the invention relates to methods of conducting a proteomics business. In one embodiment, such methods comprise identifying one or more agent (drug) that selectively suppresses toxicity to neuronal cells and licensing, to a third party, the rights for further drug development of compounds that interact with these identified agents. In other embodiments, these methods of the invention contemplate compounds (e.g., cellular components) that interact with the subject agents, or compounds that interact with the identified cellular component as described above.

In certain embodiments, the present invention provides packaged pharmaceuticals. In one embodiment, the packaged pharmaceutical comprises: (i) a therapeutically effective amount of an identified agent of the invention; and (ii) instructions and/or a label for administration of the agent for the treatment of patients having, or at risk of having, a neurodegenerative disorder such as HD. In another related embodiment, the packaged pharmaceutical comprises: (i) a therapeutically effective amount of a compound (e.g., a cellular component) that interacts with an identified agent of the invention, or a compound that interacts with an identified cellular component as described above; and (ii) instructions and/or a label for administration of the compound for the treatment of patients having or at risk of having a neurodegenerative disorder such as HD.

The present invention further provides use of any agent identified by the present invention in the manufacture of medicament for the treatment of a neurodegenerative disorder such as HD. For example, the invention provides use of a tubulin inhibitor, a known compound shown in FIG. 24, or a compound shown in FIGS. 6, 16-20, 36-37 or an analog thereof, in the manufacture of medicament for the treatment of HD.

### BRIEF DESCRIPTION OF THE DRAWINGS

FIGS. 1A-1C show modeling Htt-polyQ neurotoxicity in PC12 cells. FIG. 1(A) shows an inducible construct for production of Htt-EGFP fusion proteins. Rat neuronal PC12 cells are transfected with Htt-exon-1 constructs containing either 25 (Q25) or 103 (Q103) polyglutamine repeats (mixed CAG/CAA). FIG. 1(B) shows a cartoon of Htt-exon-1 expression in PC12 cells and the screening assay for cell viability using Alamar Blue. Induction of Htt-Q103 expression leads to the formation of perinuclear cytoplasmic inclusions (or aggresomes) of the fusion protein followed by cytotoxicity after 48 hours. Expression of Htt-Q25 remains diffuse throughout the cytoplasm and is not cytotoxic. FIG. 1(C) shows quantification of Htt-Q25 and Htt-Q103 cell viability as a measure of Alamar Blue fluorescence. Note addition of the general caspase inhibitor (BOC-D-FMK, 50 μM) rescues Htt-Q103 toxicity after a 72 hour induction with tebufenozide (Z-statistics calculated for 15000, 7500, and 3250 cells, yellow box).

FIGS. 2A-2B show the primary screening of 2,500 compounds using the Q25-Htt-exon-1 and Q103-Htt-exon-1 PC12 cell lines. The plots show two representations of thE data set. FIG. 2(A) is a histogram plot showing cell viability of Q25-Htt and Q103-Htt expressing PC12 cells after 72 hours in culture with compounds (binning interval is 400 fluorescent units). Cell viability, represented along the horizontal axis, was quantified by Alamar Blue fluorescence. FIG. 2(B) shows a scatter plot showing cell viability (Q25 versus Q103) following the 72 hour incubation in the presence of each compound (4 μg/ml). Color legend as follows: Black=compounds having either no effect, cytotoxic effect, or slight rescue of cell viability; Red=top 5 compounds that rescued Q103-induced cell death: Blue=one compound that specifically enhanced Q103-mediated cytotoxicity; Green=overlay scatter of 400 control wells without compound (average standard deviation of control wells: Q25=3845, Q103=517). Each data point in plot (B) was calculated from an average of three replicates. The standard deviations (error bars) are shown for the 7 highlighted compounds.

FIG. 3 shows the effect of cell density on the coefficient of variation (CV).

FIG. 4 shows a dose response for suppressor of mutant Huntingtin-induced toxicity.

FIG. 5 shows the tubulin inhibitors that suppressed mutant Huntingtin-induced cell death.

FIG. 6 shows the dose-response curves for the best 8 suppressors of Htt-Q103 toxicity. The viability of uninduced Q103 (Black) and tebufenozide-induced Q103-expressing cells (Red) was detected by Alamar Blue fluorescence at 72 hours postinduction (each data point is the average of 4 trials). The green inset depicts the structure of each compound.

FIGS. 7A-7F show the drug effects on cell morphology, Htt protein expression, and aggregate formation. FIGS. 7(A)-(D) show merged DIC-fluorescence and fluorescence micrographs of suppressor treated Q103 and Q25-expressing cells. Following a 42-hour induction with tebufenozide, control (untreated) Q103 cells (FIG. 7A) show a rounded detached morphology whereas SUP-1 or SUP-2-treated (FIGS. 7B and 7C, respectively, 2.5 μg/ml in DMSO) remain spread and attached to the substrate. Treatment with SUP-1 or SUP-2 does not suppress Htt-Q103 aggregate formation (FIGS. 7B and 7C). Treatment with SUP-1 or SUP-2 does not suppress Htt-Q25 expression (SUP-1 shown in D, bar-20 μm). FIGS. 7(E-F) show Htt protein expression in SUP-1 treated cells. Detergent extracts of control (DMSO) and SUP-1 treated (2.5 μg/ml for 24 and 42 hrs.) were normalized for total protein content (Bradford assay) and analyzed by Western blot (anti-EGFP of SDS-PAGE and 0.22 μm filter-trap membranes) for soluble and aggregated Htt protein. Treatment with SUP-1 does not inhibit the expression of Htt-Q25 or Htt-Q103 protein (FIG. 7E) or aggregation Htt-Q103 (FIG. 7F).

FIG. 8 is a flow diagram highlighting caspase activation pathways. Initiator caspases (e.g., caspase 8, 9, 10, and 12) can be activated by either cell surface receptor signaling or various forms of intracellular stress. Once activated, initiator caspases cleave and activate effector caspases (caspases 3, 6, and 7) which in turn target several key structural and repair proteins (e.g., Lamin A, a-Fodrin, DFF, and PARP). Additional proapoptotic and anti-apoptotic regulators (e.g., Smac/Diablo and XIAP/Survivin, respectively) have also been identified. Suppressors of Htt toxicity may function by altering the apoptosis signaling machinery.

FIGS. 9A-9C show the fluorometric assay for caspase activity in Htt-Q25 and Htt-Q103 expressing cells. FIG. 9(A) shows Caspase-3 activity measured at 15 hours post-Htt induction. Cells expressing Htt-Q103 exhibit elevated levels of caspase-3 activity over uninduced Htt-Q103 or induced Htt-Q25 expressing cells (Red, Blue and Yellow bars respectively). Suppressors of Htt-toxicity, SUP-2 and SUP-3, suppress caspase-3 activity when added to the cells in culture (Green bars). Unlike the general caspase inhibitor, BOC-D-FMK (BOC), SUP-2 and SUP-3 do not directly inhibit caspase-3 activity in solution (Purple bars, post-extraction). (B-C) Caspase-8 and caspase-9 show negligible levels of activation in the Htt-Q103 expressing cells at the same time point (FIGS. 9B and 9C, respectively). FIG. 9(D) shows Western blot detection of active caspase-3, 6, and 7. Caspases 3, 6, and 7 are differentially activated in Htt-Q103 expressing cells and this activity is suppressed by SUP-2 and SUP-3. The general caspase inhibitor (BOC) rescues cell survival by directly inhibiting the active enzymes. The Initiation Factor, eIF4E, is shown as a loading control. All proteins were detected from the same blot which was stripped and reprobed. Drug concentrations for both assays were as follows: SUP-2 (5 μM), SUP-3 (10 μM), and BOCD-FMK (50 μM).

FIGS. 10A-10C show the compound analogs to be synthesized for target identification studies. FIG. 10(A) shows a tritiated analog of SUP-1. FIG. 10(B) shows biotinylated (Blue) SUP-2 analog. FIG. 10(C) shows biotinylated SUP-3 analog with photoactivatable cross-linker (Red).

FIG. 11 is a flow chart of screening and identification of selective inhibitors of mutant N548 Htt toxicity. A cell-based assay of mutant htt toxicity was optimized for high-throughput screening. Mutant N548 cells were trypsinized, resuspended and seeded in 384-well plates using robotic advanced liquid handler (Zymark). The test compounds were robotically transferred to the wells at about 4 μg/ml concentration. Cell viability was assayed after two days using a fluorescent dye (Calcein-AM-(Molecular Probes). A “hit” was defined as any compound that enhanced fluorescence 50% above controls (vehicle treated cells); All “hits” were subsequently tested in ST14A cells to annotate nonspecific and specific inhibitors of cell death.

FIG. 12 shows the mitochondrial ETC and the site of action of inhibitors that rescue cell death in mutant Htt. The reduced intermediates NADH and FADH₂ enter the ETC at complex I and II, respectively, and electrons are then transported via electron carriers to complex III and to complex IV, where the electrons reduce oxygen to water. Cytochrome c (cyt c), the electron carrier from complex III to IV is released from the mitochondria on apoptotic signaling and causes activation of the apoptotic machinery. Rotenone blocks substrate entry at complex I but not at complex II. Antimycin prevents the ETC at Complex III thereby blocking both complex I and II.

FIG. 13 shows a dose response curve for selective cell death rescue by mitochondrial ETC inhibitor (antimycin A) and MT depolymerizing agent (colchicine). Plotted on the horizontal axis is the drug concentration while the vertical axis shows cell viability assayed as fluorescence intensity of a cell viability dye in N548 mutant (blue) and ST14A (black). The fluorescence intensity was normalized to signal in DMSO treated cells that was arbitrarily given a value of 100%. The values represent the mean±S.D. of an experiment in triplicate and are representative of multiple independent experiments (>5).

FIGS. 14A-14B show disruption of MT network by MT inhibitor. Immunofluorescence α-tubulin in N548 mutant cells. FIG. 14(A) shows the MT network in untreated cells. FIG. 14(B) shows a diffuse cytoplasmic MT after treatment with microtubule inhibitor (podophyllotoxin).

FIGS. 15A-15B show the criteria for identification of candidates involved in cell death rescue by MT depolymerization. FIG. 15(A) shows cell lysates prepared from mutant N548 cells in the presence or absence of MTI will be immunoprecipitated with a htt specific antibody. Any proteins that change association with htt proteins upon MTI treatment will be candidates (shaded). FIG. 15(B) show, in a similar approach, proteins that show differences in association with tubulin in mutant N548 and ST14A will be candidates.

FIGS. 16A-J show the compounds and their analogs identified in the PC12 cell assay system.

FIGS. 17A-17B show a summary of representative compounds and their analogs identified in the PC12 cell assay system.

FIG. 18 shows the compounds or analogs identified using the ST14A cell assay system.

FIG. 19 shows the compounds and their analogs identified in the ST14A cell assay system.

FIG. 20 shows the structures, efficacy, and effective concentrations of various classes of selective hits in N548 mutant and PC12 HD model.

FIGS. 21 A-G show characterization and optimization of a striatal neuronal HD assay for screening. FIG. 21(A) shows increasing cell numbers plated in 6 replicates in a 384-well plate. After 6 h, cell fluorescence was determined by the calcein AM assay and is shown as average±one S.D. FIG. 21(B) shows the inverse relationship between CV and cell seeding density. CV at increasing cell densities in a 384 well plate was determined using 6 replicates per cell density. FIG. 21(C) shows the time course of calcein fluorescence signal. 1500 cells were plated/well and subjected to calcein AM assay. Fluorescence was measured over 5 h. (FIG. 21(D)). T-ag protein decreases at 39° C. ST14A and N548 mutant cells were incubated at 33° C. or 39° C. for 6 h, and cellular T-ag protein levels were determined by western blotting. FIG. 21(E) shows that low serum concentration decreases viability of N548 mutant cells. 1500 cells/well were plated in 384-well plates with medium containing a range of serum concentration (0-5% IFS). Cell viability was assayed after 3 d incubation at 33° C. (open squares) or 39° C. (diamonds). The data is the average±SD of 9 replicates and is representative of two experiments. FIG. 21(F) shows relative protection from cell death of N548 WT cells compared to N548 mutant cells under serum deprivation. 1500 cells/well of each cell line were incubated at 39° C. for 3 d in 0.5% IFS and viability assayed by calcein assay. Data is the average±SD of at least 20 replicates and is representative of three independent experiments. FIG. 21(G) shows expression of mutant htt as assessed in ST14A, N548 mutant and N548 WT cells using a polyQ specific antibody (top panel) and an anti-htt antibody (Mab2166) (middle panel). Tubulin was used as a loading control.

FIGS. 22A-B show HTS and hit identification. FIG. 22(A) is a flowchart of the hit discovery process. FIG. 22(B) is the screening data for the NINDS library compounds. 1,040 compounds arrayed in 384-well plates and one DMSO plate were assayed in triplicate. One plate was assayed on the day of cell seeding as a control for complete rescue (yellow triangles). A 50% increase in signal above the median plate signal in two of three replicate wells was set as a threshold to identify hits (horizontal bar). A hit that enhances signal in triplicate wells to levels similar to cells on day of plating is circled.

FIGS. 23A-N show the identification of non-selective and mutant htt-length selective inhibitors of cell death. FIGS. 23(A)-(B) show pan-caspase inhibitor BOC-D-fmk inhibits caspase activity and prevents cell death non-selectively. FIG. 23(A) shows ST14A, N548 mutant and N548 WT incubated for 6 h in 0.5% IFS containing media at 39° C., with or without BOC-D-fmk (50 μM in ST14A and N548 mutant cells). Activation of individual caspases was monitored fluorometrically by measuring the cleavage of specific peptide substrates. Fluorescence in each sample was normalized to protein and represented relative to the fluorescence in N548 WT cells. The results are the average±SD of one experiment performed in triplicate. FIG. 23(B) shows adose dilution of BOC-D-fmk tested in ST14A and N548 mutant cells where cell death was induced by 0.5% IFS containing media at 39° C. The viability based on calcein AM in BOC-D-fmk treated cells is expressed relative to vehicle (DMSO) treated cells. The results are the average±SD of an experiment performed in triplicate. FIG. 23(C) shows the phase contrast images of morphology of N548 mutant and ST14A cells after 2 days in serum deprived media with DMSO (0.1%) (FIGS. 23(D)-(L)). Structures, dose response of cell death rescue in a panel of 3 different length mutant htt-expressing cell lines and ST14A cells, and phase contrast images of N548 mutant and ST14A cells treated with 3 compounds representative of each of the 3 subclasses that are selective for different htt lengths. FIGS. 23(D)-(F) show N548 mutant selective compound, revertin-4, (G-I) N63 and N548 mutant selective compound, revertin-8 and (FIGS. 23(J)-(L)) N548 and FL-mutant selective compound, revertin-14. All phase contrast images were from cells treated with 8 μg/ml of each compound. FIG. 23(M) shows cell viability determined by trypan blue exclusion assay in ST14A and N548 mutant cells after 2 days in SDM with DMSO (0.1%), BOC-D-fmk (50 μM), revertin-4, revertin-8 or revertin-14 (8 μg/ml each) and is the average±SD of an experiment in duplicate. At least 1000 cells were counted for each sample. (N) Western blotting for the N548 mutant htt expression after 20 hr treatment with DMSO (0.1%), revertin-4, revertin-8 or revertin-14 (8 μg/ml each). Tubulin served as a loading control.

FIG. 24 shows a table of the compounds with known biological mechanisms identified as non-selective protective agents in the ST14A assay.

FIG. 25 shows a table of specificity classification of the revertins identified in the ST14A assay.

FIGS. 26A-C show the i-Identification of novel microtubule inhibitors based on selectivity profiling. FIG. 26(A) shows the structure of compound revertin-22 and revertin-23. FIG. 26(B) shows the selectivity profile for cell death rescue by microtubule inhibitor (MTI), colchicine, and revertins-22 and 23. FIG. 26(C) shows revertin-22 and colchicine depolymerize microtubules in N548 mutant cells. Micrographs of β-tubulin immunofluorescence in N548 mutant cells treated with DMSO (0.1%), colchicine (400 nM) or revertin-22 (4 μ/ml) for 8 h.

FIGS. 27A-F show that novel compounds selectively prevent neuronal death and inhibit caspase cleavage in N548 mutant cells. FIG. 27(A) shows structures of two novel compounds rev-1a and rev-2. FIG. 27(B) shows phase contrast images of N548 mutant cells under serum deprivation (0.5% IFS) after 2 days with DMSO (0.1%), rev-la (10 μg/ml) or rev-2 (12 μg/ml) treatment. FIG. 27(C) shows a dose response of cell viability (calcein AM) for rev-1a in the three mutant htt expressing cell lines and ST14A cells. FIG. 27(D) shows a dose response of cell viability based on trypan blue exclusion, in mutant N548 cells after 2 days under serum deprivation conditions after rev-1a or rev-2 treatment. The data represents the average±SD of an experiment performed in duplicate and is representative of atleast 2 independent experiments. FIG. 27(E) shows the expression of mutant htt and T antigen upon treatment with rev-2 (8 μg/ml) and rev1a (10 μg/ml) for 20 hours was determined by western blotting. Tubulin served as a loading control. FIG. 27(F) shows the effect of rev2 and rev1a on cleavage of caspase-3 and 7. Mutant N548 or ST14A cells were incubated in 10% IFS (Ser) or serum deprived media with different concentrations of rev-2 and rev1a or DMSO (−) for 20 hours and then harvested. Equal total cellular protein was loaded on a 4-20% SDS PAGE and subjected to western blotting for cleaved caspase-3 and caspase-7. Tubulin served as a loading control. The data is representative of two independent experiments.

FIGS. 28A-C show that rev-1a and rev-2 enhance neuronal survival in PC12 HD model. FIG. 28(A) demonstrates that rev-2 shows morphological rescue of PC12-Q103 cells induced to express httQ103. Phase contrast images of uninduced, induced (+Teb) and induced cells treated with rev-2 (12 μg/ml) or BOC (50 μM) after 42 hr of induction. FIG. 28(B) shows rescue of PC12 HD toxicity by revertin-1c (100 μg/ml) and revertin-2 (12 μg/ml). Rescue was expressed relative to rescue by BOC-D-fmk (50 μM) that was a 100%. The results are the average±SD of two experiments performed in triplicate. FIG. 28(C) shows a dose dilution of rev-2 assayed for rescue of cell viability in induced PC12-Q103 cells by trypan blue exclusion assay. Data is represented as relative cell viability, with viability of induced cells set as 0% and that of uninduced as 100% viability. The results are the average±S.D. of an experiment performed in triplicate and is representative of 3 independent experiments.

FIGS. 29A-D show that rev-1a and rev-2 suppress neuronal cell death in a C. elegans. HD model. FIG. 29(A) show photomicrographs of ASH neuron in the C. elegans as viewed under a fluorescent microscope in a 1-day old animal (arrow). ASH death was assayed by a loss of GFP expressing ASH neurons in a 3-day old animal (right panel). FIG. 29(B) shows that revertin-2 (0.8 mg/ml) and revertin-1a (1 mg/ml) enhance ASH neuronal cell survival in a C. elegans HD model. ASH neuronal cell survival was assayed at 2 d after compound treatment. The results are the average±SD of three independent experiments (n=50 animals, 100 neurons). The rescue was significant *(two tail t-test, p<0.02). FIG. 29(C) shows the time course of ASH neuronal death. ASH neuronal cell survival was assayed at 1 and 3 d and the data is the average±SD of 5 independent experiment (50 animals, 100 neurons were scored per experiment). FIG. 29(D) shows rescue of ASH neuronal death by rev-1a (1 mg/ml), rev-2 (0.8 mg/ml) and trichostatin A (TSA 1 mM). The results are the average±SD of three independent experiments (50 animals and 100 neurons were scored per experiment); the rescue was significant in each case*(two tailed student's t-test, p<0.02).

FIG. 30 shows that the compounds of R-1 series rescue MSN degeneration in HD brain slice assay. Rat brain slices (postnatal day10) were co-transfected with a reporter plasmid (YFP) along with human htt-Q73-CFP or CFP. Transfected brain slices were treated with DMSO (C), BOC-D-fmk (50 μM), R-1a or R-1b and MSN degeneration was assessed at day 5. Healthy MSNs were counted per striatum and is shown as the average±S.E. from 7 or more brain slices per treatment. The rescue was significant (p<0.05, ANOVA and Dunnet's posthoc comparison test) for BOC, R-1a (5 μg/ml and 10 μg/ml) and R-1b (5 μg/ml and 20 μg/ml). The results are representative of three experiments for R-1a and two for R-1b.

FIG. 31 shows optimization of Z′ factor using the Alamar Blue assay. 1,500 cells/well were seeded in a 384 well plate (Costar 3712) in 57 μl of media (DMEM supplemented with 0.1 mM sodium pyruvate, 2 mM glutamine, penicillin/streptomycin (50 units/ml; 50 μg/ml) with 0.5% IFS) and incubated at 39° C. After 48 h, 20 μl of 40% alamar blue (Biosource, CA) in media was added per well and cells incubated for 24 h at 39° C. Cell viability was assayed by measuring alamar blue reduction (ex 530/em 590 nm) in a plate reader (Perkin Elmer Victor3). The Z′ factor was calculated for 3 independent experiments using BOC-D-fmk (50 μM) as the positive signal and DMSO treated cells as negative signal (n=20 wells/treatment).

FIG. 32 shows the testing effects of compounds on N548-mutant htt transgene expression. N548-mutant cells were incubated with the hit compounds R1-27 (1b, 1c to 27, rotenone (Rot), colchicie (Col), valinomycin (Val)) at a concentration ˜2 times the EC50 for 24 h. Total protein extracts were subjected to western blotting for mutant htt expression using the MAB2166 antibody. ST14A cells were included as negative controls for transgene expression (ST14) and DMSO treated cells [C] as controls for vehicle treatment. Blots were probed for tubulin that served as a loading control. The lowest panel shows repeats of a few compounds that were not clearly represented in the top 3 westerns panels or appear to decrease (16) and also to show the reproducibility.

FIG. 33 shows a summary of novel compound activity in 3 HD models. EC50—effective concentration 50; TC50—toxic concentration 50. ND—none detected at highest concentration tested. *PC12 Efficacy (percent rescue relative to BOC that was set a 100%). § These compounds show activity in ST14A but are more efficacious in N548-mutant cells.

FIG. 34 shows activity testing in N548 mutant cells of compounds previously identified in other HD assays.

FIG. 35 shows the medicinal chemistry profiles of novel hits.

FIG. 36 shows a summary of the structure activity relationship for R1 compound series. Activity was calculated as fold increase above the plate median (control). Any compound showing an increase in activity 1.5 fold above control was considered active. All compounds were tested at ˜4 ug/ml in triplicate.

FIG. 37 shows a summary of the structure activity relationship for R2 compound series. All analogs were tested in a 13 point, 2-fold dose dilution series from 20 ug/ml to ˜10 ng/ml and were assayed in triplicate.

### DETAILED DESCRIPTION OF THE INVENTION

The ability of genotype-selective compounds to serve as molecular probes is based on the premise of chemical genetics—that small molecules can be used to identify proteins and pathways underlying biological effects (Schreiber, Bioorg. Med. Chem. 1998, 6: 1127-1152; Stockwell, Nat Rev Genet 2000, 1: 116-25; Stockwell, Trends Biotechnol 2000, 18: 449-55). For example, the observation that the natural product rapamycin retards cell growth made possible the discovery of the mammalian Target of Rapamycin (mTOR) as a protein that regulates cell growth (Brown et al., Nature 1994, 369: 756-758; Sabatini et al., Cell 1994, 78: 35-43). The present invention combines these two approaches, chemical and molecular genetic, to discover pathways affected by mutations associated with neurodegenerative disorders such as HD.

### Compounds of the Invention

The present invention's studies demonstrate that it is possible to identify compounds with increased potency and activity in the presence of specific genetic elements. For example, work described herein provides a novel systematic testing using more than 23,000 compounds and one or more genetic elements associated with a neurodegenerative disorder such as HD. In another embodiment, a high-throughput assay in a striatal neuronal cell culture model of HD was developed to screen 47,000 compounds for the ability to suppress cell death.

In certain embodiments, inhibitors (suppressors) of mutant huntingtin-induced neuronal cell death have been identified using the screening methods of the invention. By screening a library of biologically active compounds, compounds were identified that selectively prevent mutant huntingtin-induced death of neuronal cells, but do not act on neurons lacking mutant huntingtin protein. In certain alternative embodiments, a small number of compounds were identified that increase viability of mutant huntingtin-expressing neuronal cells as well as wild-type huntingtin-expressing cells and/or parental cells. Certain compounds identified by the present invention prevented polyQ-toxicity in an htt-length-dependent manner while others were effective in an htt-length-independent manner, suggesting that mutant htt toxicity may involve multiple mechanisms distinct for different htt length fragments.

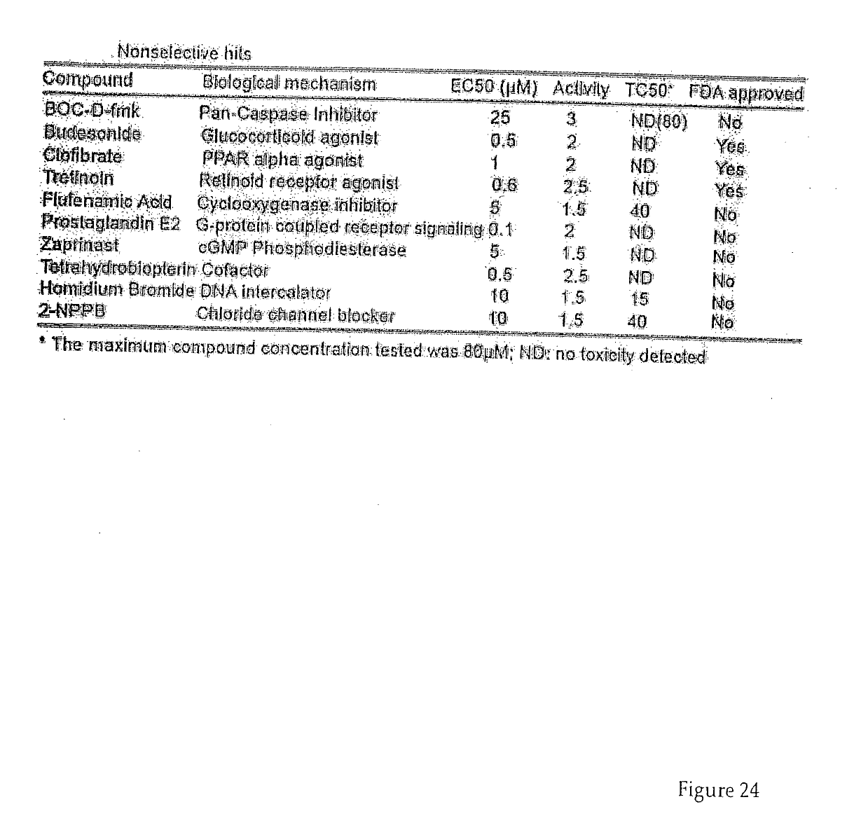

For example, the suppressors of mutant huntingtin-induced neuronal cell death include, but are not limited to, tubulin inhibitors (e.g., colchicines, podophyllotoxin, vincristine, and vinblastine; FIG. 5), thiomuscimol, N-P-tosyl-L-valine chloromethyl ketone, parthenolide, forskolin, 1-methylisoguanosine, dihydrocytocholasin-B, 2-phenyaminoadenosine, and the nonselective suppressors of cell death shown in FIG. 24 (BOC-D-fmk, Budesonide, Clofibrate, Tretinoin, Flufenamic acid, Prostaglandin E2, Zaprinast, Tetrahydrobiopterin, Homidium Bromide, and 2-NPPB).

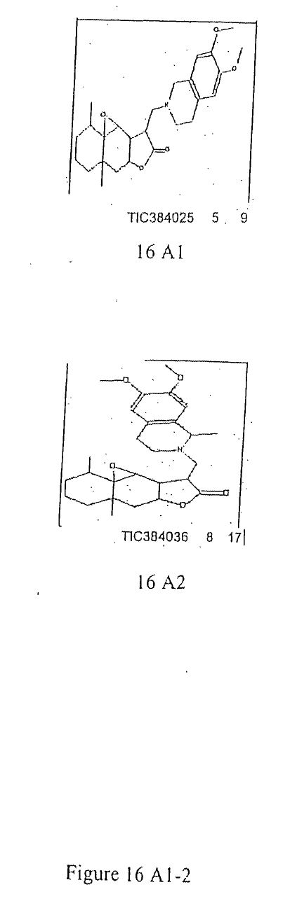

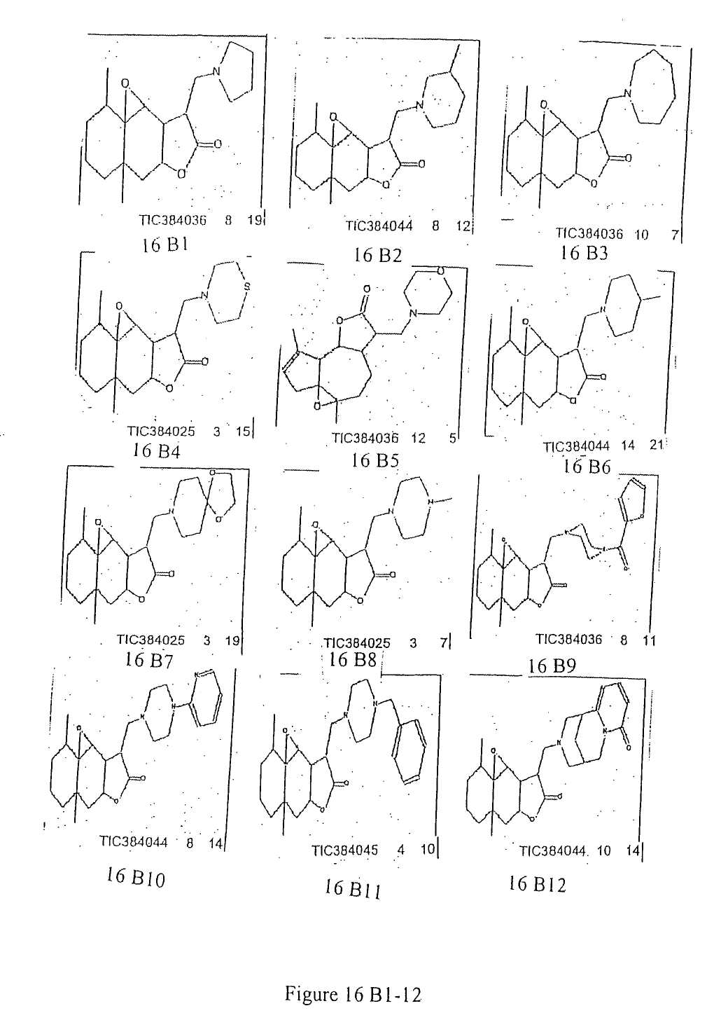

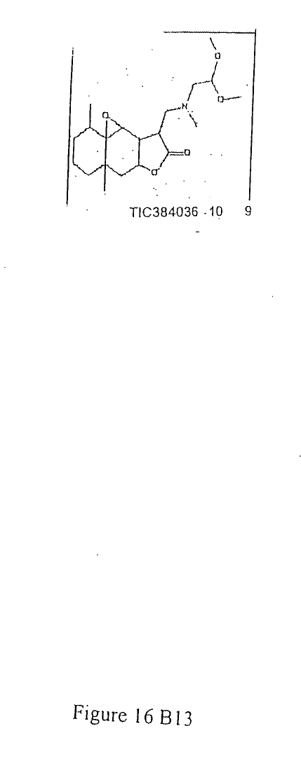

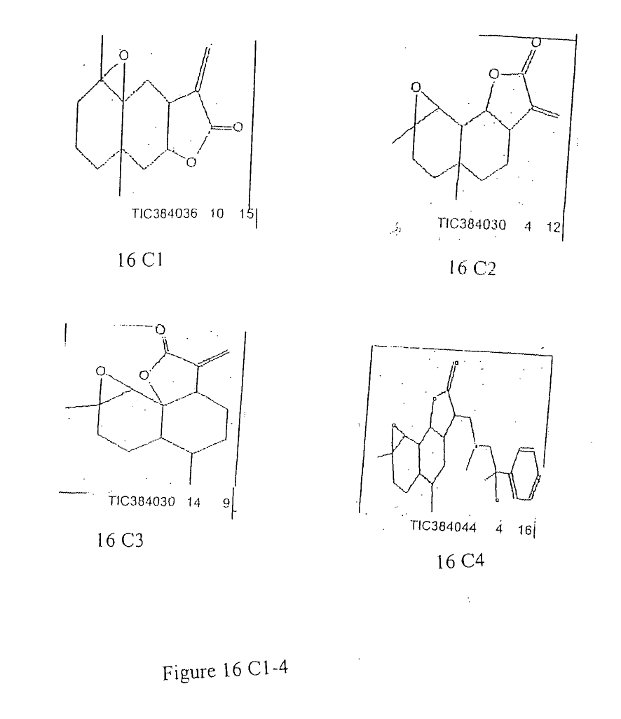

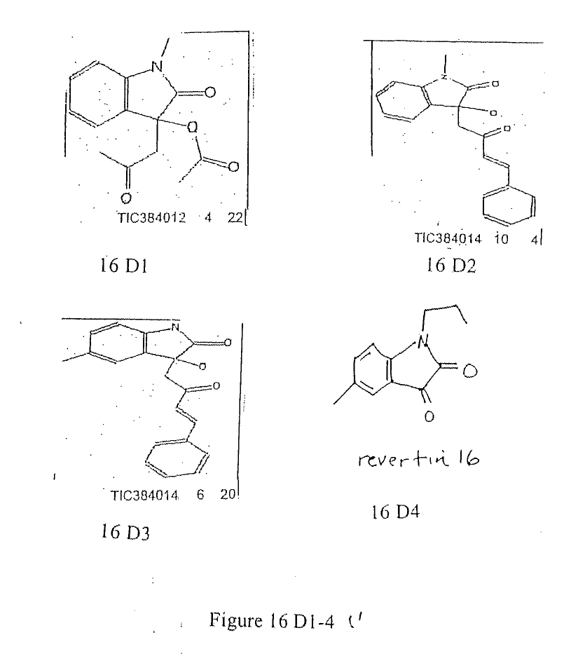

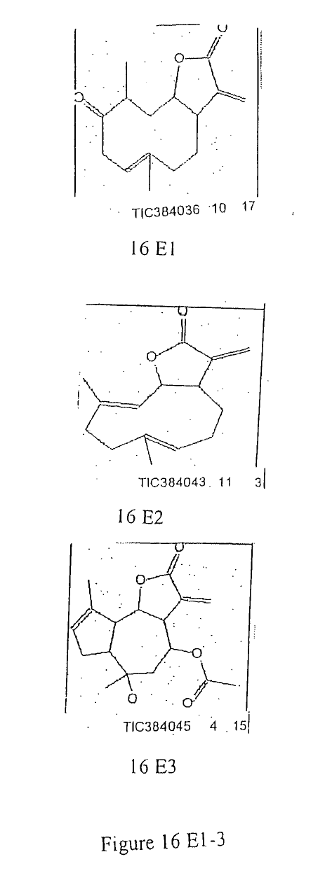

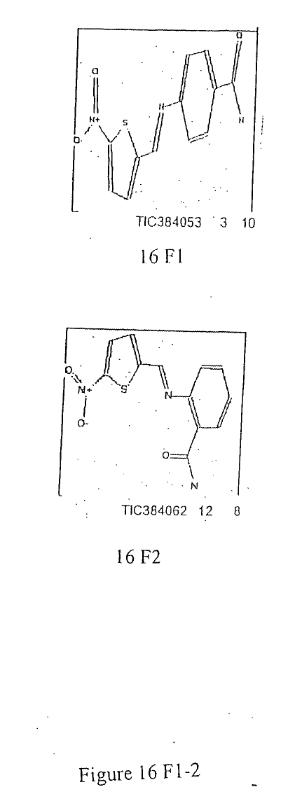

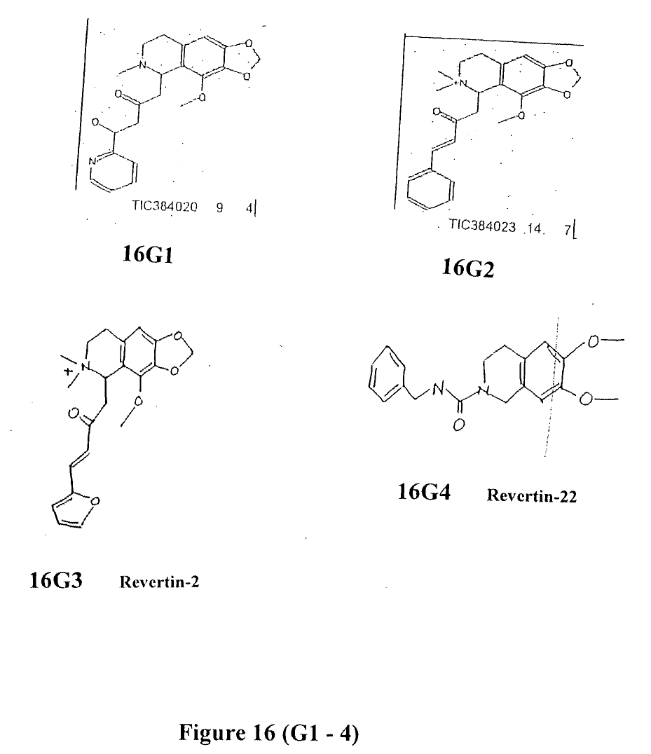

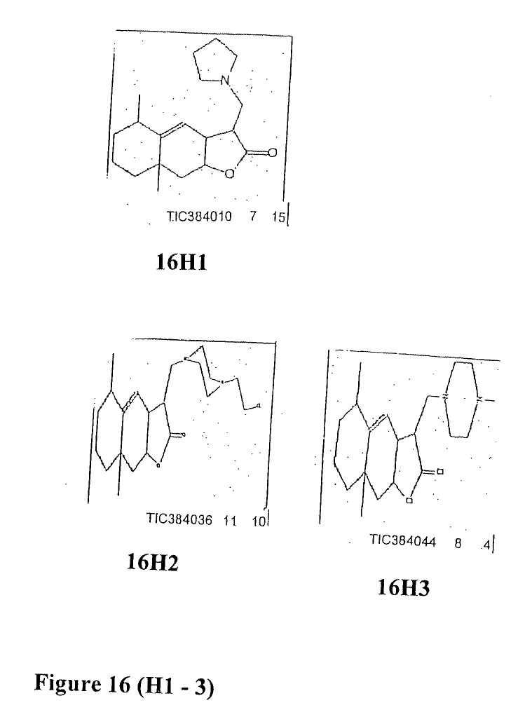

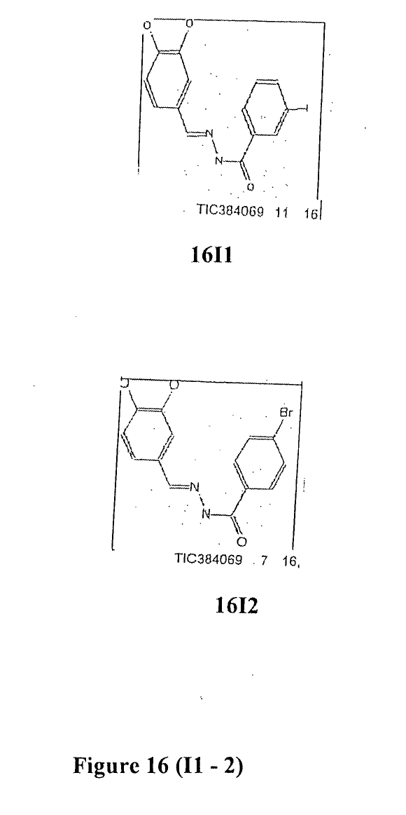

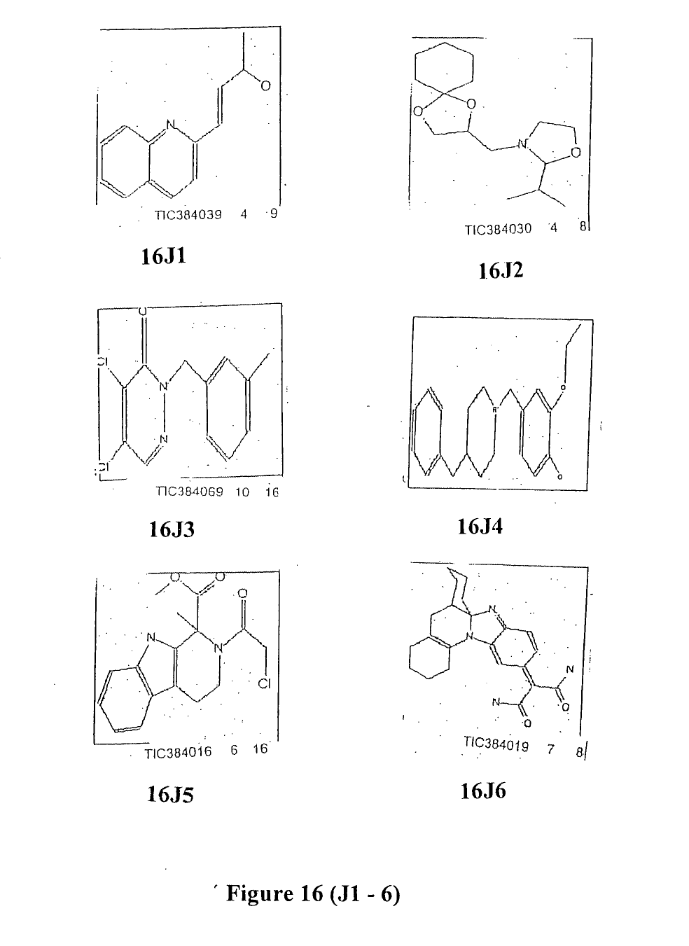

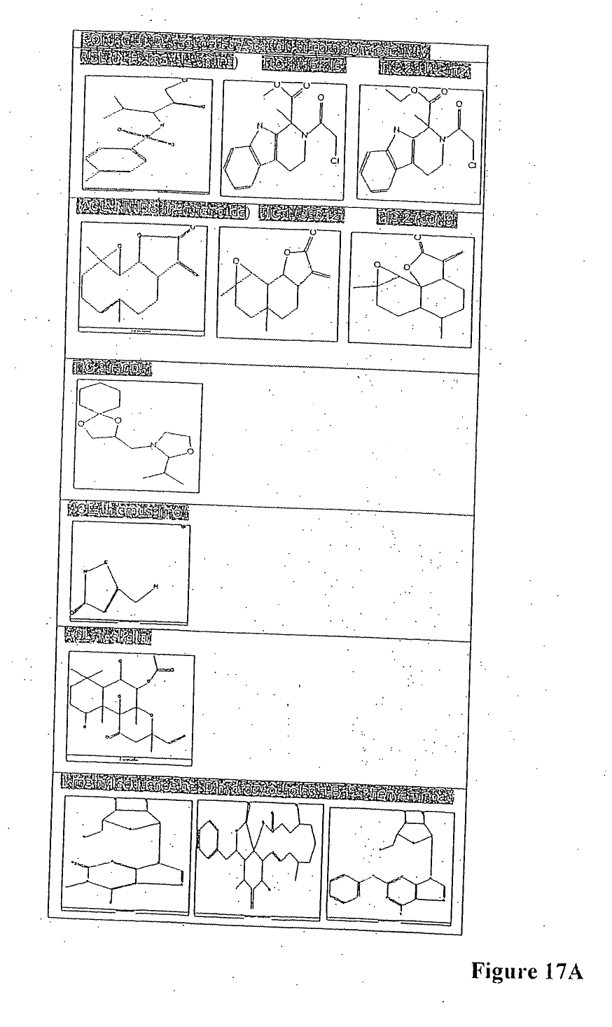

Further, the present invention provides isolated compounds having a formula shown in FIGS. 16-20 and 36-37. Preferably, these compounds suppress toxicity to neuronal cells. For example, some of the subject compounds contain a chloromethyl ketone group, an alpha methyl lactone group, an enone group, or a three-ringed structure.

In certain embodiments, the present invention contemplates analogs or derivatives of the subject compounds as described above. To illustrate, the chloromethyl group of a subject compound can be replaced with a methyl or difluoromethyl group. Exemplary analogs of the invention include, but are not limited to, tritiated analogs, biotinylated analogs, and analogs with photoactivatable cross-linkers (see, e.g., FIG. 10). It is understood that methods of making structural analogs and derivatives of a compound are known and routine in the art. The toxicity-suppressing activity of the analogs and derivatives can be readily assayed by the methods described in the invention.

In certain embodiments, the genotype-selective compounds of the invention (e.g., anti-HD agents) can be any chemical (element, molecule, compound, drug), made synthetically, made by recombinant techniques or isolated from a natural source. For example, these compounds can be peptides, polypeptides, peptoids, sugars, hormones, or nucleic acid molecules (such as antisense or RNAi nucleic acid molecules). In addition, these compounds can be small molecules or molecules of greater complexity made by combinatorial chemistry, for example, and compiled into libraries. These libraries can comprise, for example, alcohols, alkyl halides, amines, amides, esters, aldehydes, ethers and other classes of organic compounds. These compounds can also be natural or genetically engineered products isolated from lysates or growth media of cells—bacterial, animal or plant—or can be the cell lysates or growth media themselves. Presentation of these compounds to a test system can be in either an isolated form or as mixtures of compounds, especially in initial screening steps.

### Exemplary Compounds of the Invention

The following is a description of non-limiting examples of compounds and classes and subclasses of compounds which are encompassed by the present invention. In specific non-limiting embodiments described below, the terms alkyl or alkenyl (taken alone or in compound terms) refer to structures containing between 1-6, between 1-5, between 1-4 or between 1-3 carbon atoms, and cyclic compounds may contain 3-12 or 4-10 or 4-7 atoms.

In a first set of non-limiting embodiments, the compounds of the invention may be represented by related formulas I or II (where the line drawn from the substituent and crossing the cyclohexane ring indicates that ring structures A and B can share any bond with the cyclohexane ring and where, in formula II, ring B may share a bond with ring A):

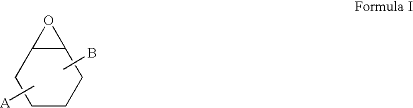

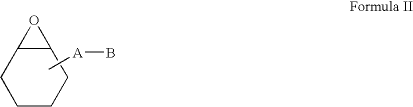

where A is a substituted or unsubstituted cycloalkyl, aryl, or heterocyclyl;

where B is a substituted tetrahydrofuran-2-one:

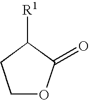

where R¹ may be an alkylheterocyclyl group comprising one or two nitrogen atoms and optionally further comprising between 0-2 further heteroatoms which may be O or S; and in a set of non-limiting embodiments is alkylheterocyclyl, the heterocyclyl group comprising one or more N atom and optionally further comprising between 0-2 further heteroatoms which may be O or S. In specific, non-limiting embodiments, R¹ is (CH₂)ₙ-D, where n=1-3 and D may be substituted or unsubstituted quinoline; or may be substituted or unsubstituted isoquinoline; or may be substituted or unsubstituted piperidine; or may be substituted or unsubstituted piperazine; or may be substituted or unsubstituted pyrrolidine.

Alternatively, R¹ may be an alkyl amine where the nitrogen of the amino group may be linked to one or two H, alkyl, or alkoxy groups (e.g., FIG. 16B13), or R¹ may be an alkenyl group (e.g., FIG. 16C1-3).

In one non-limiting set of embodiments, compounds of formula I may have formula III:

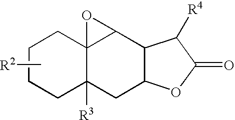

where R² is H, methyl, alkyl, or cycloalkyl (and where the line drawn from the substituent crossing the ring indicates that the substituent may be linked to any of the carbons in the ring);

where R³ is H, methyl, alkyl, or cycloalkyl;

where R⁴ is alkylheterocyclyl, the heterocyclyl group comprising one or more N atom and optionally further comprising between 0-2 further heteroatoms which may be O or S. In specific, non-limiting embodiments, R⁴ is (CH₂)ₘ-E, where m=1-3 and E may be substituted or unsubstituted quinoline; or may be substituted or unsubstituted isoquinoline; or may be substituted or unsubstituted piperidine; or may be substituted or unsubstituted piperazine; or may be substituted or unsubstituted pyrrolidine. In non-limiting embodiments, where E is a substituted piperazine, the substituent may be alkyl (e.g., FIG. 16B8), alkylaryl (e.g., FIG. 16B11) or alkylheteroaryl (e.g., FIG. 16B10).

In one specific set of non-limiting embodiments, R⁴ may be:

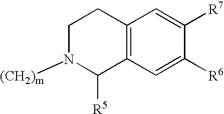

where R⁵, R⁶ and/or R⁷, which may be the same or different, may be H, alkyl, alkoxy, or alkoxyalkyl (see, e.g., FIGS. 16A1 and 16A2).

For further non-limiting examples of compounds of Formula I, see FIGS. 16B9, 16B12 and 16B13.

For non-limiting examples of compounds of Formula II, see FIGS. 16C1-4.

In a second set of non-limiting embodiments, the compounds of the invention may be represented by Formula IV:

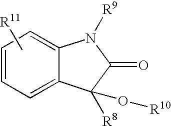

where R⁸ may be absent (in which case the bond to oxygen is a double bond) or may be H, alkyl, alkenyl, alkylcarbonylalkyl, alkenylcarbonylalkyl, alkylcarbonylalkenyl, alkylaryl, or alkylcarbonylalkenylaryl, and may comprise one or more double bond in a carbon chain and may comprise one or more heteroatoms such as O, N or S;

where R⁹ may be H or alkyl, e.g., methyl, ethyl, propyl or butyl;

where R¹⁰ may be absent or may be H, alkyl, alkylcarbonylalkyl, alkylhydroxyl, or alkylhydroxylalkyl; and

where R¹¹ may be H or alkyl (e.g., methyl, ethyl, or propyl) (where the line drawn from the substituent crossing the ring indicates that the substituent may be linked to any of the carbons in the ring).

Non-limiting examples of compounds of Formula IV are depicted in FIGS. 16D1-4.

In a third set of non-limiting embodiments, the compounds of the invention may be represented by Formula V:

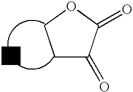

where F is a cycloalkyl or heterocycloalkyl group comprising one or two fused ring structures, one or both of which may comprise one or more double bond, optionally bearing one or more substituent which may be alkyl, hydroxy, keto, epoxy, halo, alkylcarbonyl, and/or alkylcarboxy. In particular embodiments, the ring or fused ring structures of F together contain between 9 and 11, or 10, carbon atoms, not considering substituents.

Specific non-limiting examples of compounds of Formula V are depicted in FIG. 16E1-3.

In a fourth set of non-limiting embodiments, the compounds of the invention may be represented by Formula VI:

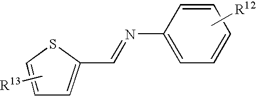

where substituent R¹² may be H, alkyl, amide, alkylamide; alkylcarbonyl, alkoxycarbonyl, or sulfonyl (and where the line drawn from the substituent crossing the ring indicates that the substituent may be linked to any of the carbons in the ring); and

where substituent R¹³ may be H, alkyl, NO₂, alkylcarbonyl, alkoxycarbonyl or sulfonyl (and where the line drawn from the substituent crossing the ring indicates that the substituent may be linked to any of the carbons in the ring).

Specific non-limiting examples of compounds of Formula VI are depicted in FIG. 16F1-2.

In a fifth set of non-limiting embodiments, the compounds of the invention may be represented by Formula VII:

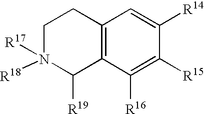

where R¹⁴ and R¹⁵ may be the same or different, and may be H, alkyl or oxy or alkoxy or alkoxycarbonyl, and may be joined to form a ring structure, for example where R¹⁴ and R¹⁵ together form an oxymethoxy ring with C6 and C7 of isoquinoline; or where R¹⁴ and R¹⁵ together form a furan ring with C6 and C7 of isoquinoline;

where R¹⁶ may be H, alkyl, alkoxy, alkoxyalkyl, or alkoxycarbonyl, for example, but not by way of limitation, methoxy, ethoxy, or propoxy;

where R¹⁷ may be H or alkyl, for example, but not by way of limitation, methyl or ethyl;

where R¹⁸ may be absent or may be H or methyl; and

where R¹⁹ may be alkylcarbonylalkyl, alkylcarbonylaryl, alkylcarbonylalkenylaryl, or amidoalkylaryl or amidoalkylheteroaryl or where, in a non-limiting set of embodiments, R¹⁹ is (CH₂)ₒcarbonyl-G-J, where o=1-3 and G is alkyl, alkenyl, or alkylhydroxy, and J is substituted or unsubstituted aryl or heteroaryl, for example phenyl, pyridine, pyrazine, pyrimidine, pyrrole, furan, isoxazole, or isothiazole.

Specific non-limiting examples of compounds of Formula VII are depicted in FIG. 16G1-4.

In a sixth set of non-limiting embodiments, the compounds of the invention may be represented by Formula VIII:

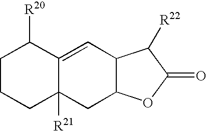

where R²⁰ may be H, alkyl, or alkoxy, for example methyl or ethyl;

where R²¹ may be H, alkyl, or alkoxy, for example methyl or ethyl; and

where R²² may be H, alkyl, alkoxy, alkylcycloalkyl, alkylaryl, alkylheteroaryl, or alkylheterocyclyl, wherein in non-limiting embodiments the heterocyclic group may be a substituted or unsubstituted piperidine, piperazine, or pyrrolidine, or a substituted or unsubstituted phenyl, pyrazine, pyridine, pyrimidine, pyrrole or furan.

Specific non-limiting examples of compounds of Formula VIII are depicted in FIG. 16H1-3.

In a seventh set of non-limiting embodiments, the compounds of the invention may be represented by Formula IX:

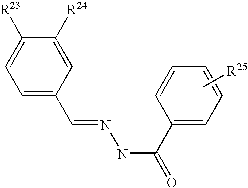

where R²³ and R²⁴ may be the same or different and may be H, alkyl, hydroxy, or alkoxy; and

where R²⁵ may be H, alkyl, alkoxy, hydroxy, or halo (including fluoro, bromo, or iodo).

Specific non-limiting examples of compounds of Formula IX are depicted in FIG. 16I1-2.

Additional non-limiting examples of compounds of the invention are depicted in FIG. 16J1-6.

In an eighth set of non-limiting embodiments, the compounds of the invention may be represented by Formula X:

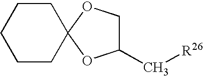

In Formula X,

R²⁶ may be a heterocyclyl group preferably a 4, 5, 6 or 7-membered ring comprising N and in certain specific embodiments further comprising O in an epoxide linkage, such as but not limited to morpholine, methypyridine, or oxazole, optionally substituted with R²⁷ which may be C₁₋₄ alkyl (e.g. methyl, ehtyl, propyl, isopropyl) or aryl (e.g., phenyl).

In one specific set of non-limiting embodiments, R²⁶ may be a substituted oxazole:

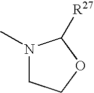

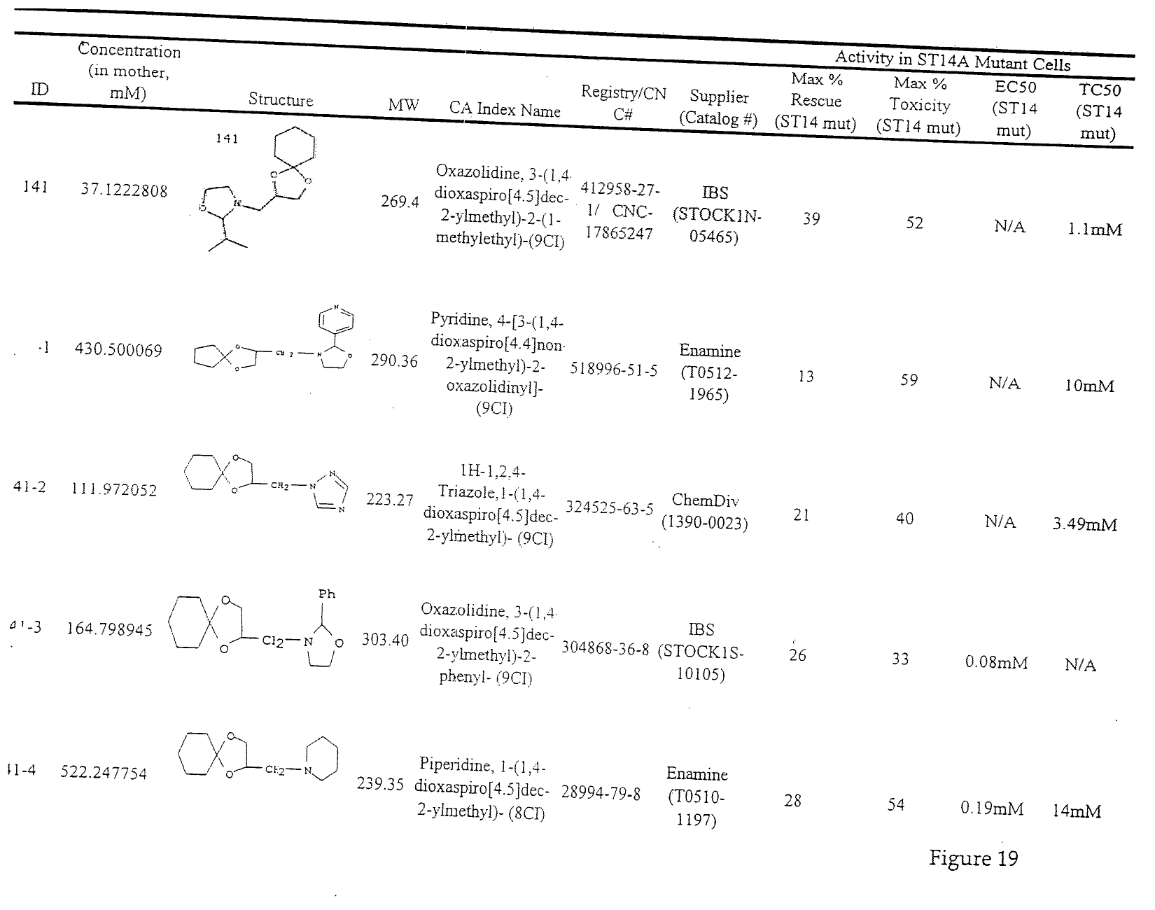

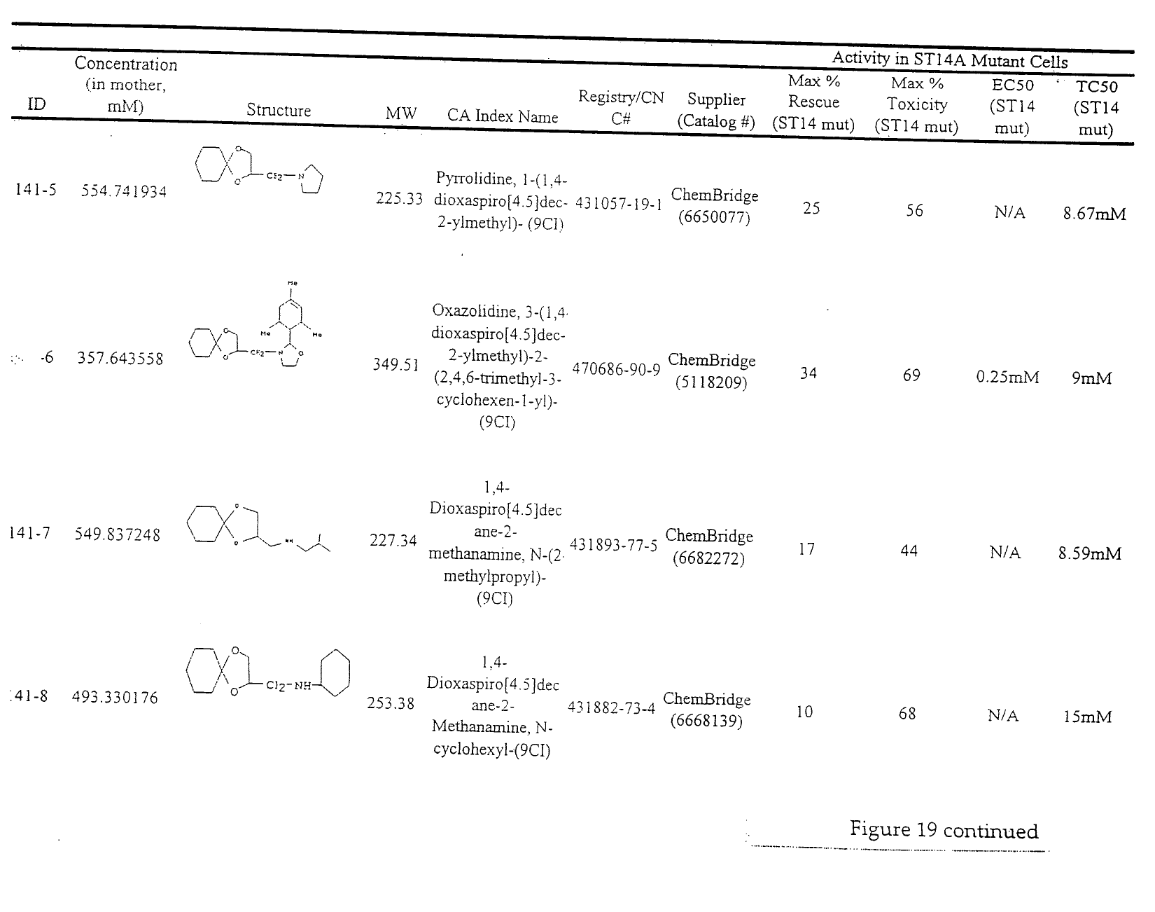

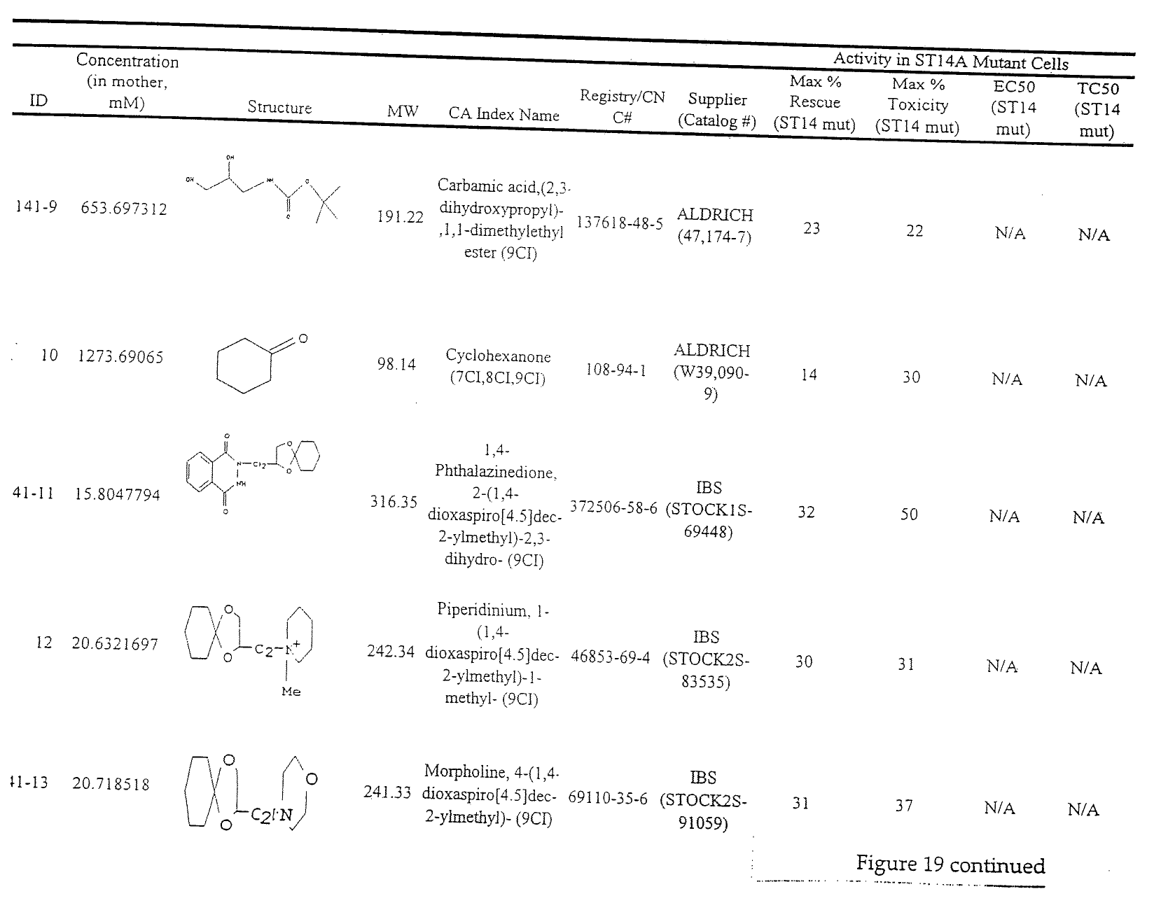

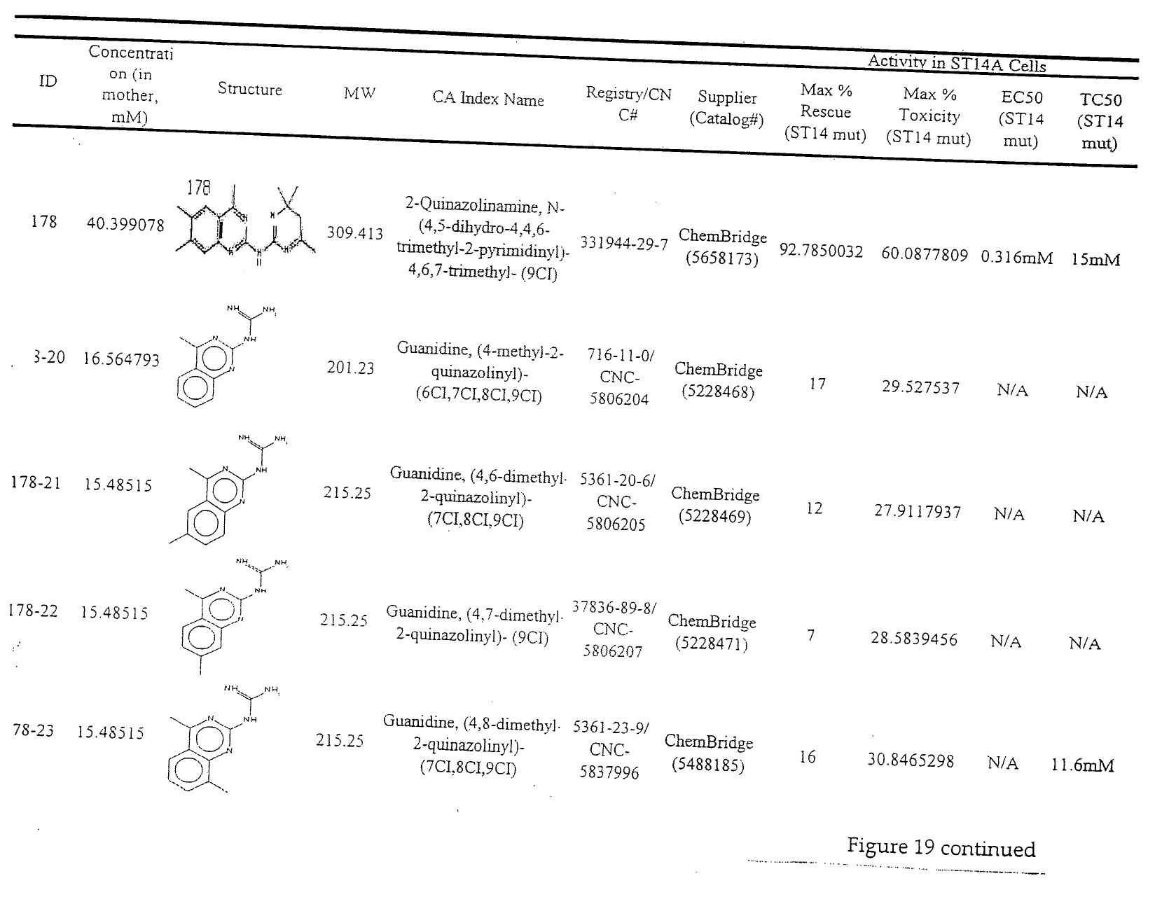

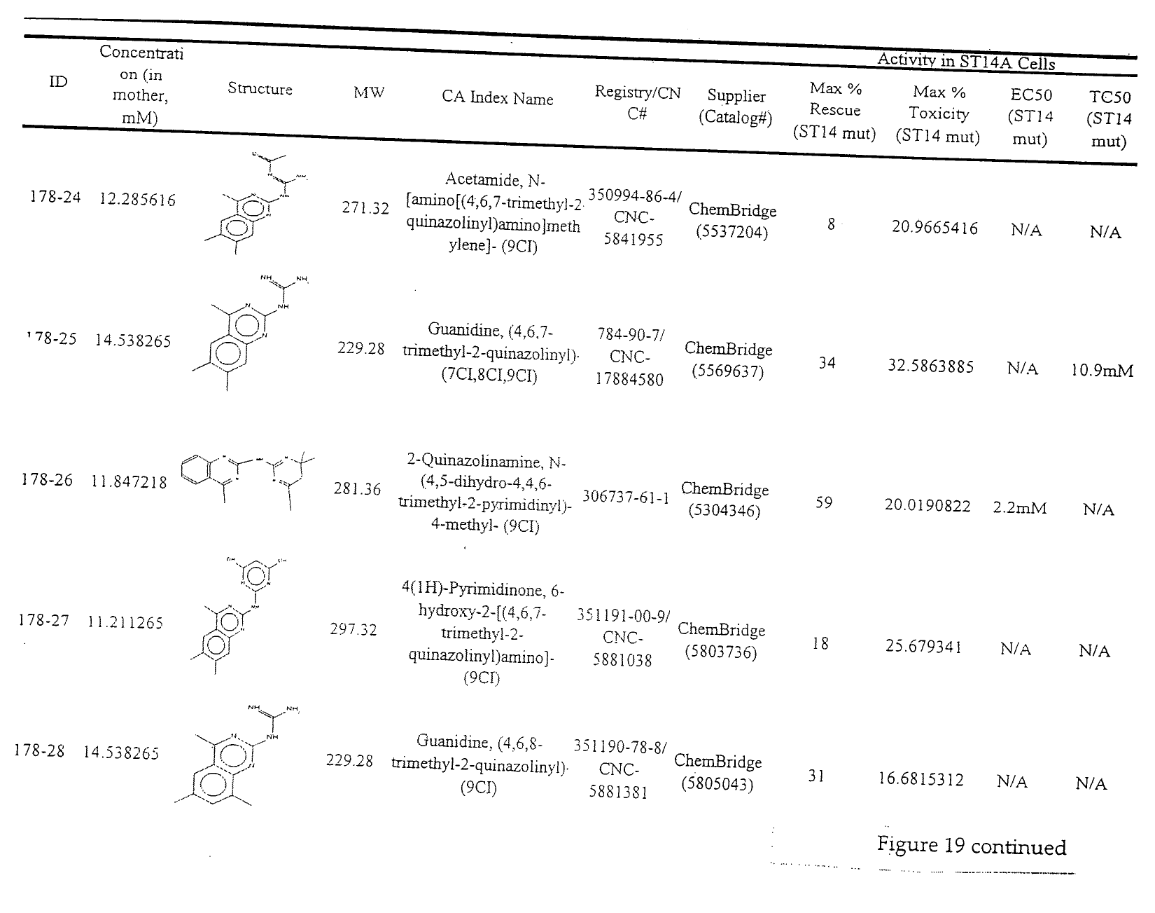

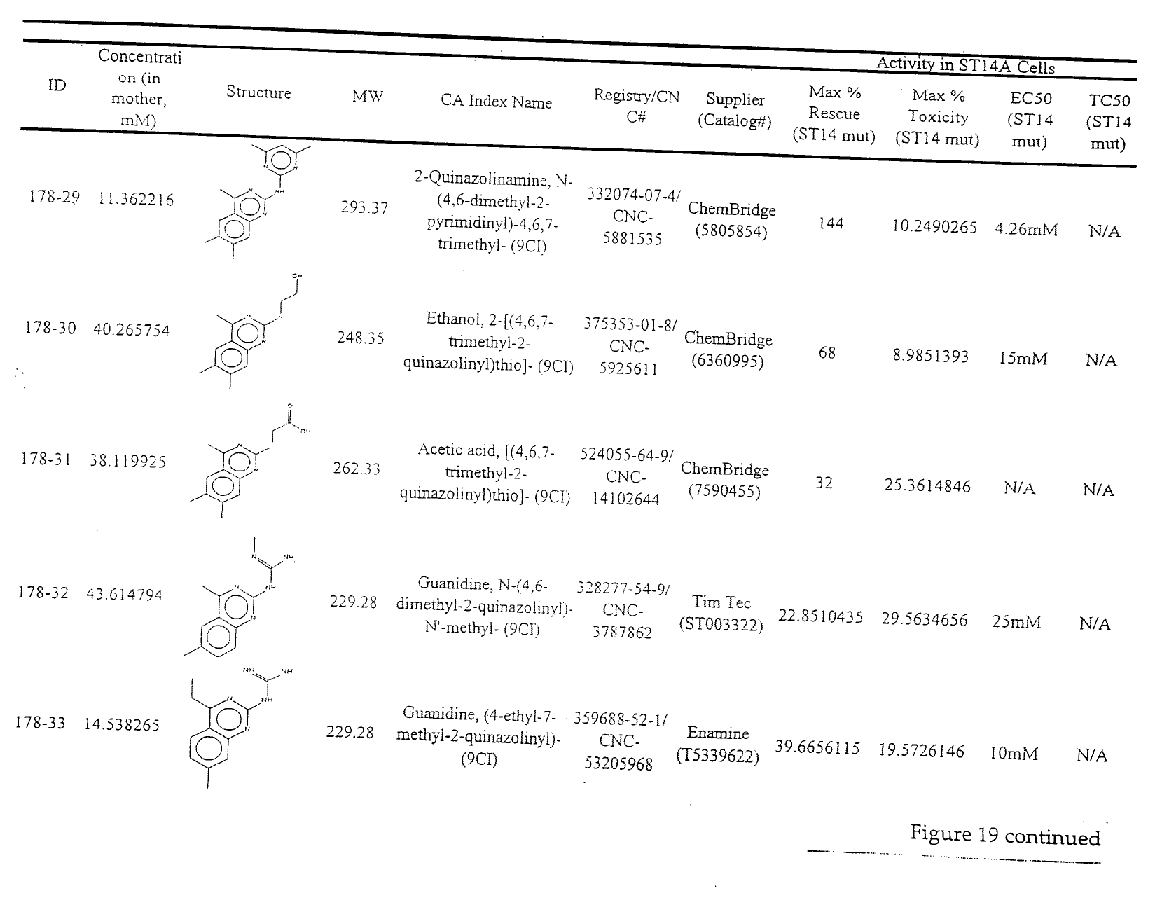

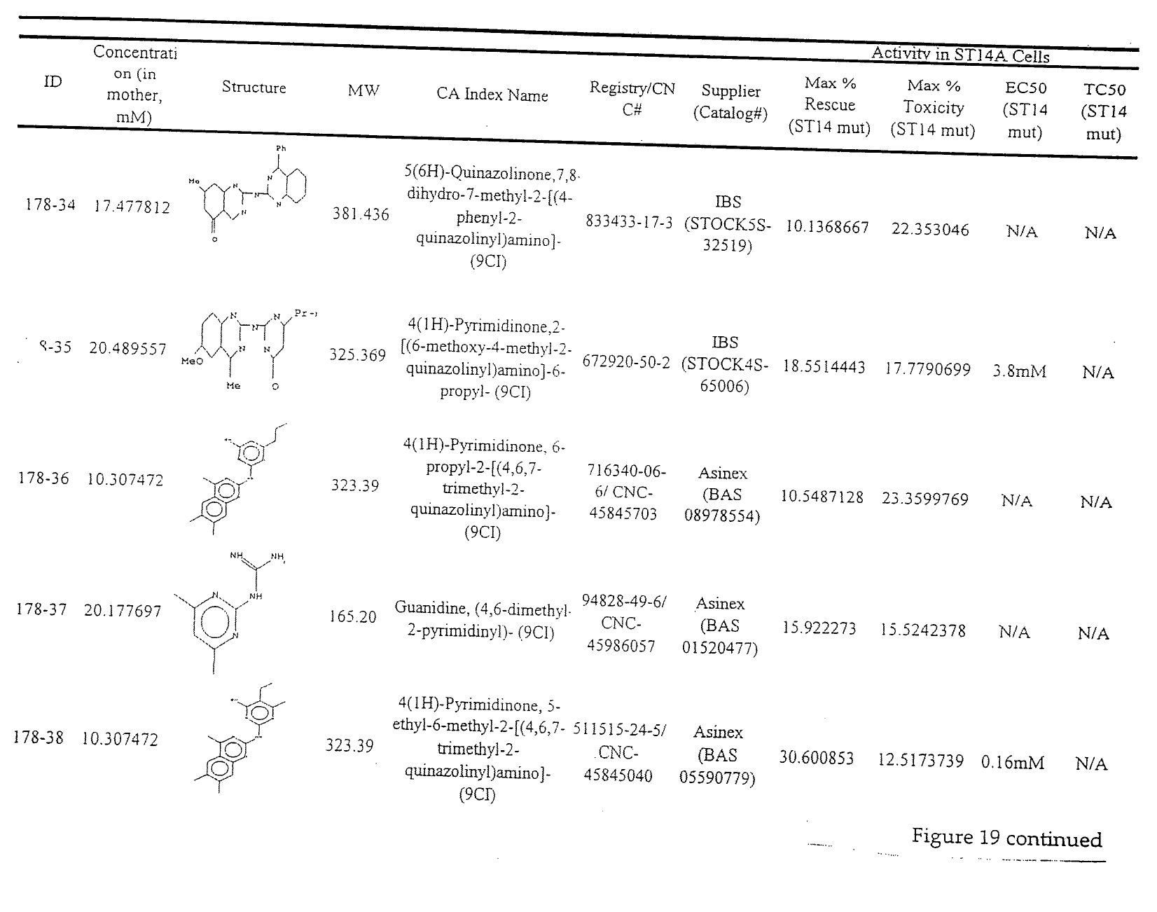

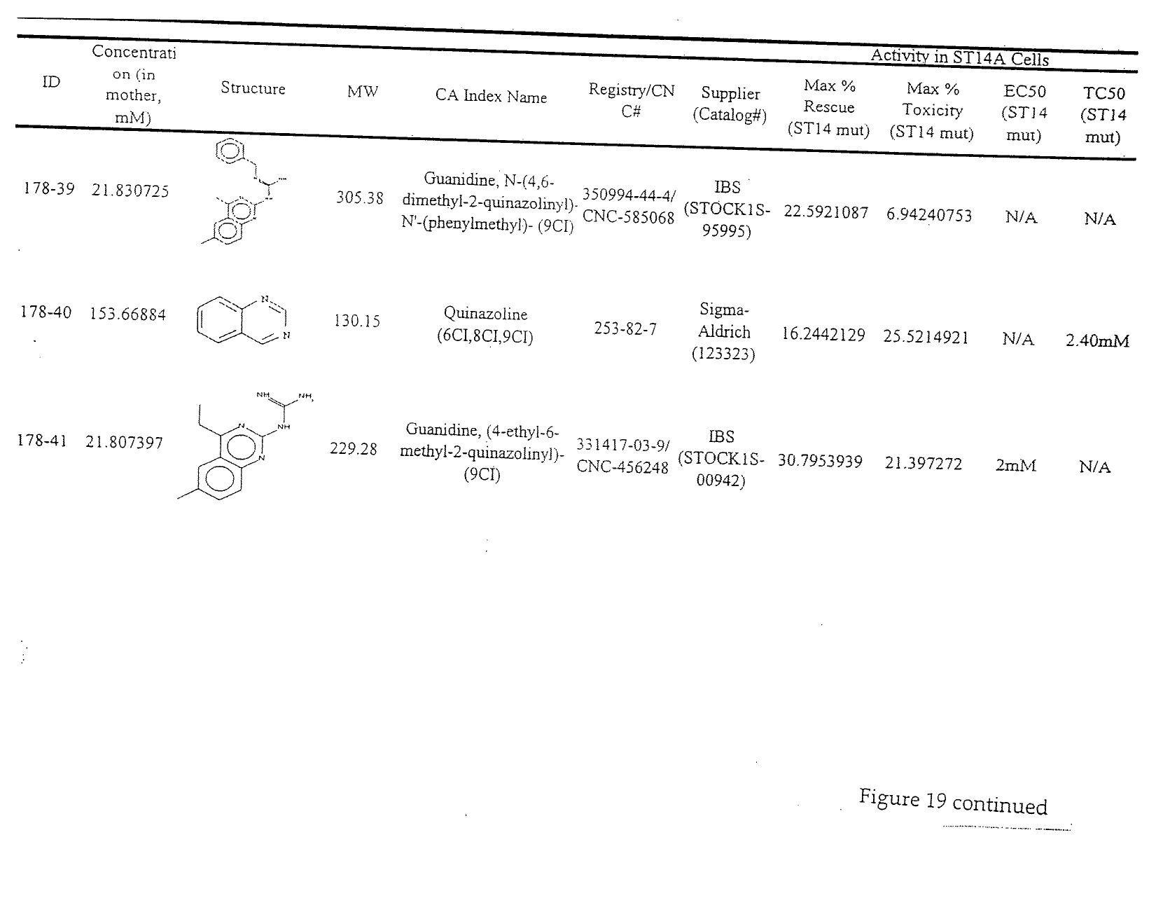

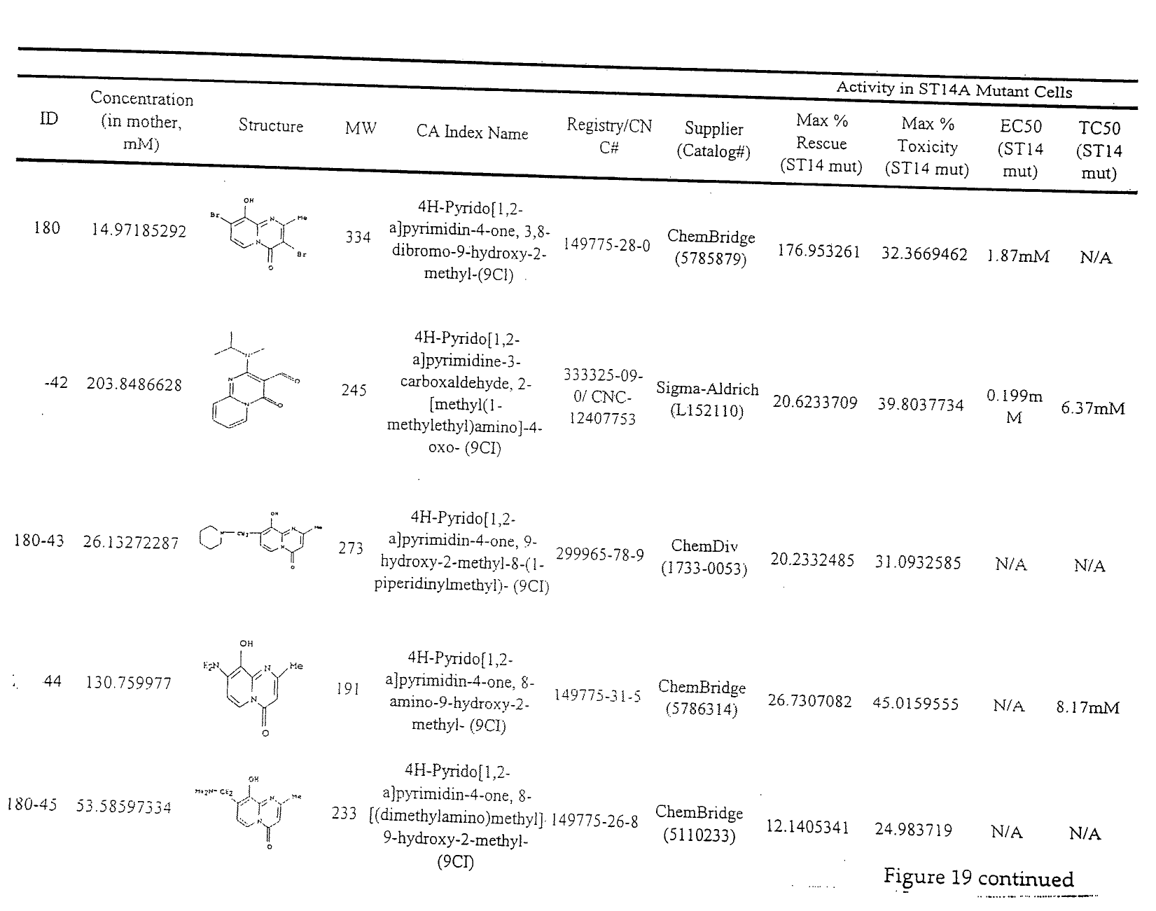

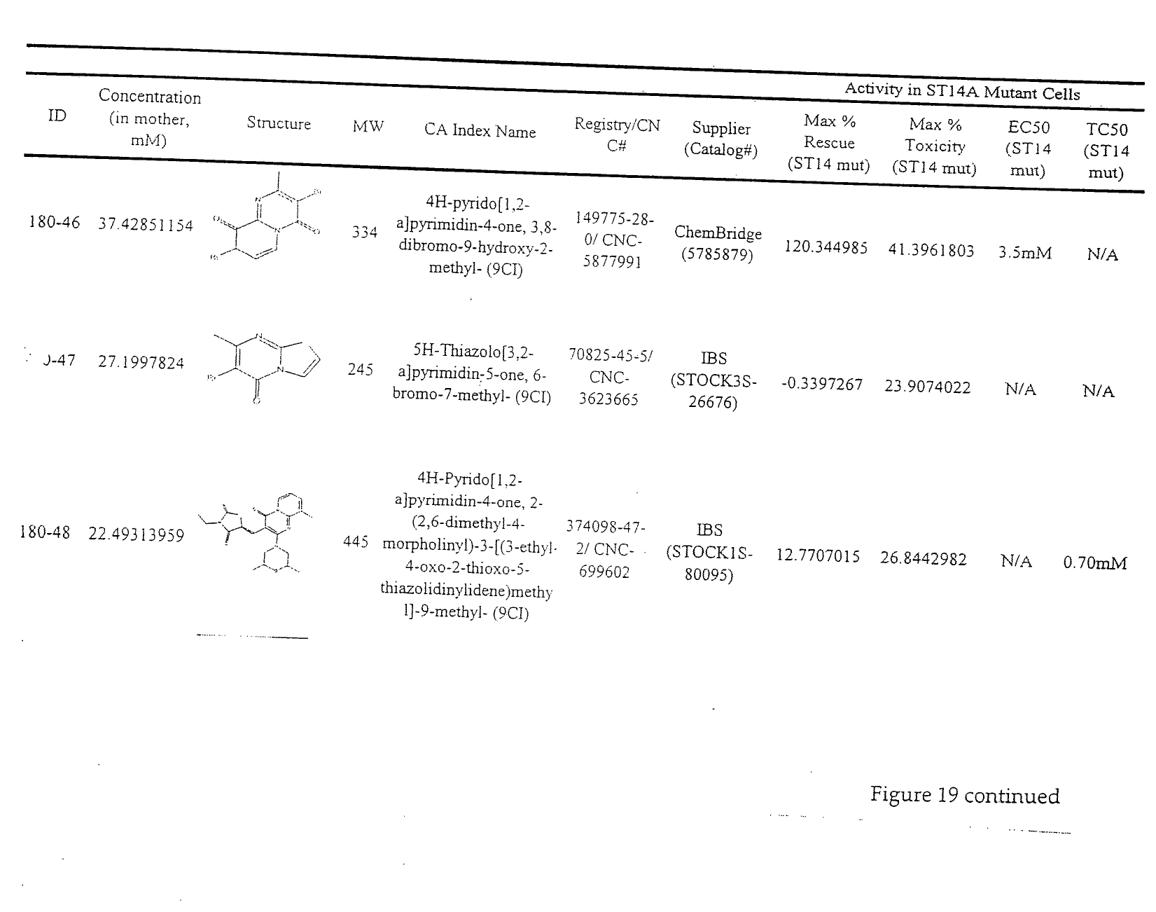

Examples of compounds having Formula X are shown in FIG. 19. Preferred examples of compounds of Formula X include compound 141, 141-3, 141-9, 141-12, and 141-13, as shown in FIG. 19. In specific non-limiting embodiments, the local concentration of a Formula X compound at a neuron to be treated may be between about 1-50 micromolar, or between about 2 and 30 micromolar, or between about 0.05 and 15 mM. Without being bound by any theory, it is believed that compounds of series 141 may be relatively likely to cross the blood brain barrier.

In a ninth set of non-limiting embodiments, the compounds of the invention may be represented by Formula XI:

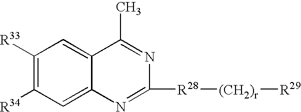

In formula XI,

r=0 or 1-2 and is preferably 0;

R²⁸ may be NH or S, and is preferably NH;

R²⁹ may be hydroxy, or a 4-7 member ring heterocyclyl group, where the heteroatom is preferably nitrogen, such as but not limited to substituted pyrimidine, or a carbamidoyl group (—C(═NH)—NH2, also referred to as “amidino” or “amidine”) which is optionally substituted, where the substitutent may be on N¹ or N², said carbamidoyl substituent being selected from the group consisting of C₁₋₄ alkyl (e.g. methyl, ethyl, propyl, isopropyl), aryl (e.g., phenyl), or C₁₋₄ alkylaryl.

In one preferred non-limiting embodiment, R²⁹ may be:

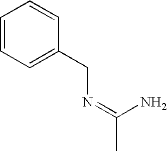

In another preferred non-limiting embodiment R²⁹ may be:

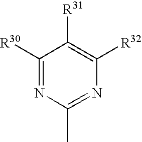

R³⁰ may be H, hydroxy, (C₁₋₄)alkyl, dimethyl or methyl, ethyl;

R³¹ may be H, hydroxy, (C₁₋₄)alkyl, dimethyl or methyl, ethyl;

R³² may be H, hydroxy, (C₁₋₄)alkyl, dimethyl or methyl, ethyl.

In one preferred, non-limiting embodiment, R³⁰ is methyl, R³¹ is H, and R³² is methyl;

In another preferred, non-limiting embodiment, R³⁰ is hydroxy, R³¹ is ethyl, and R³² methyl;

In another preferred, non-limiting, embodiment, R³⁰ is a dimethyl, R³¹ is absent, and R³² methyl.

R³³ may be H or (C₁₋₄)alkyl, and is preferably methyl;

R³⁴ may be-H or (C₁₋₄)alkyl, and is preferably methyl.

In one preferred, non-limiting embodiment, R³³ and R³⁴ are both methyl.

Examples of compounds having Formula XI are shown in FIG. 19. Preferred examples of compounds of Formula XI include compounds 178-26, 178-29, 178-30, 178-38, and 178-39, as shown in FIG. 19. In specific non-limiting embodiments, the local concentration of a Formula XI compound at a neuron to be treated may be between about 0.1 to 20 mM or between about 0.1 and 10 mM.

In a tenth set of non-limiting embodiments, the compounds of the invention may be represented by Formula XII:

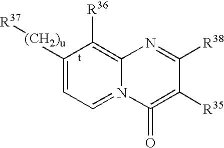

In Formula XII,

R³⁵ may be H, (C₁₋₄)alkyl, I, F or Br, and is preferably Br;

R³⁶ may be hydroxy or keto, and is preferably hydroxy;

t may be a single bond or a double bond, and is a single bond when R³⁶ is keto and a double bond when R³⁶ is hydroxy.

u=0 or 1, and is preferably 0; and

R³⁷ may be Br, F or I or a 4-7 member heterocyclyl group optionally substituted with (C₁₋₄)alkyl, such as but not limited to piperidine, and is preferably Br.

Examples of compounds having Formula XII are shown in FIG. 19. Preferred examples of compounds of Formula XII include compounds 180, 180-43, and 180-46, as shown in FIG. 19. In specific non-limiting embodiments, the local concentration of a Formula XII compound at a neuron to be treated may be between about 0.1 to 20 mM or between about 0.1 and 10 mM.

In another set of non-limiting embodiments, the compounds of the invention may be represented by Formula XIII:

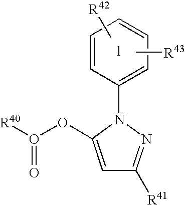

In Formula XIII,

R⁴⁰ may be a substituted or unsubstituted aromatic, substituted or unsubstituted diaromatic, or C₁-C₁₀ alkyl. Substituent groups include, but are not limited to, H, halogens, C₁-C₄ alkyl groups, alkoxy groups. The number of substituents may be one, two or more than two. Preferably, the substituent groups are flourine or chlorine, trifluoromethyl, C₁-C₄ alkyl groups, or C₁-C₄ alkoxy groups. Preferably, R⁴⁰ is a substituted or unsubstituted phenyl or napthyl for example, fluorophenl, trifluoromethylphenyl or (di-trifluoromethyl)phenyl.

R⁴¹ may be a (C₁₋₄)alkyl, alkoxy, aromatic ring, or dimethyl group.

Ring 1 may be additionally substituted, wherein R⁴² and R⁴³ may be H or a halogen, preferably flourine or chlorine. R⁴² and R⁴³ may be the same or may be different substituent groups. In a specific embodiment, Ring 1 has one flourine substituent. In another embodiment, Ring 1 has two chlorine substituents.

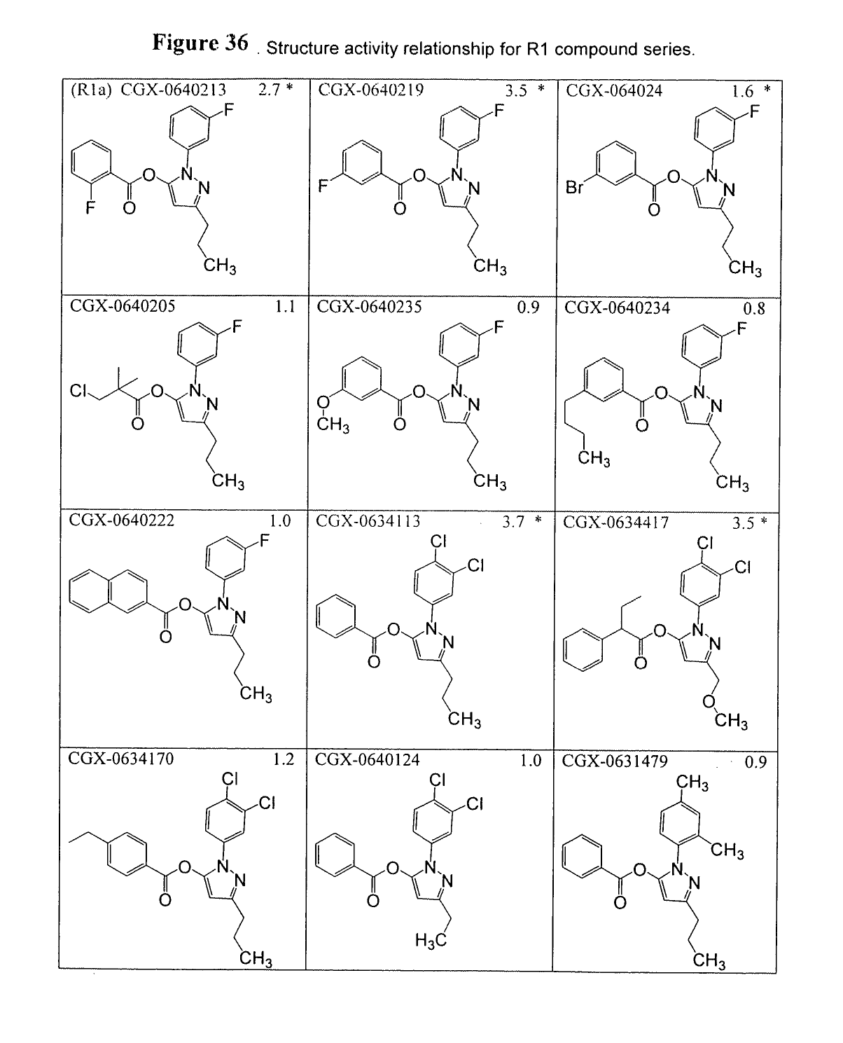

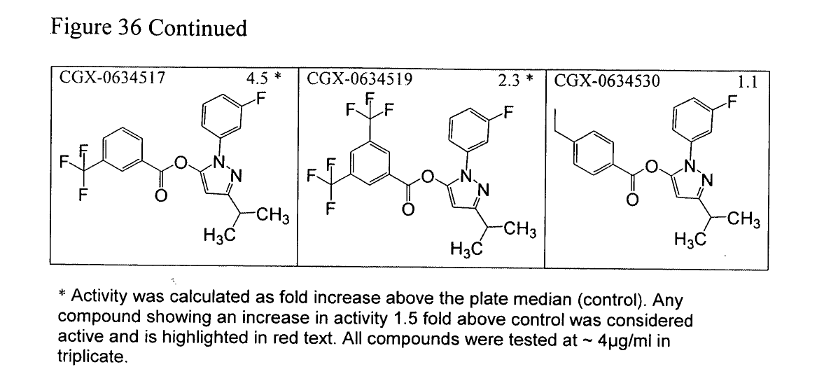

Additional examples of compounds of Formula XIII are exemplified in FIG. 36.

In another non-limiting embodiment, the compounds of the invention may be represented by Formula XIV:

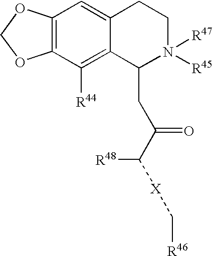

In Formula XIV,

R⁴⁴ and R⁴⁵ may be C₁-C₄ alkoxy groups.

“—X—” may be a single or double bond or an amide bond.

R⁴⁶ may be absent or, for example, one of the following substituent groups: 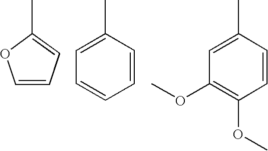
or may be a substituted aromatic rin or heterocyclic (optionally aromatic ring);

R⁴⁷ may be absent or may be hydrogen or methyl (in which case the N is a quaternary ammonium ion); and

R⁴⁸ maybe C or N or O.

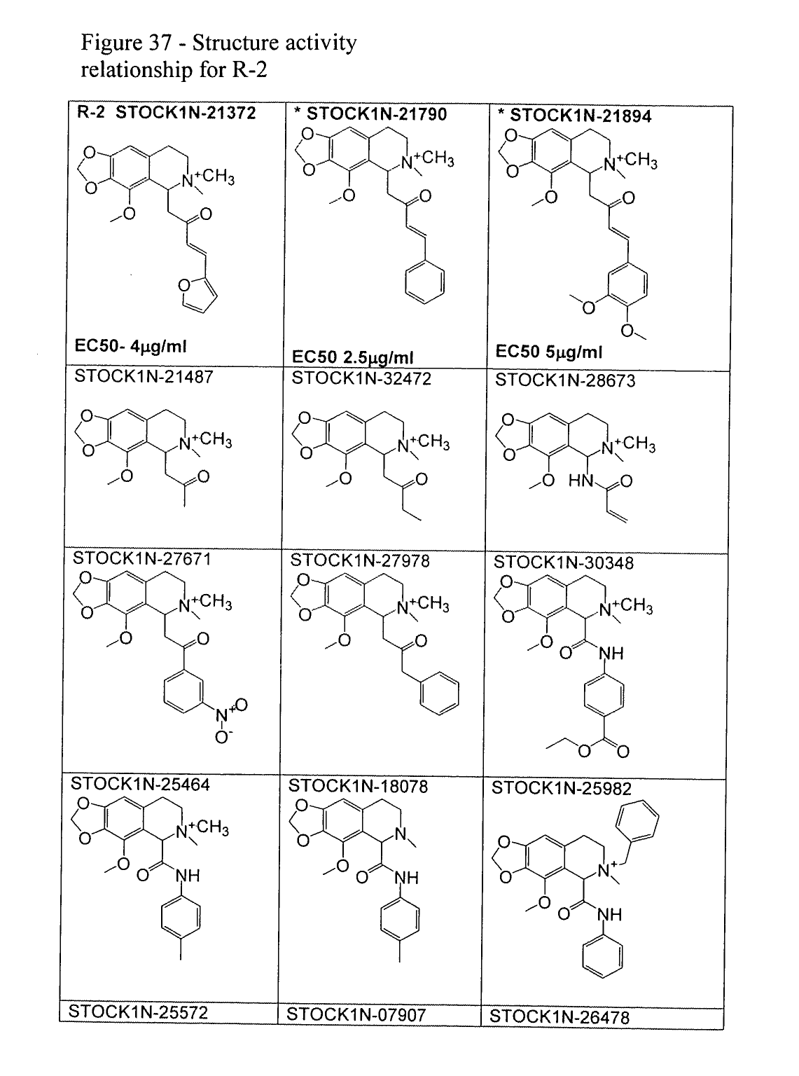

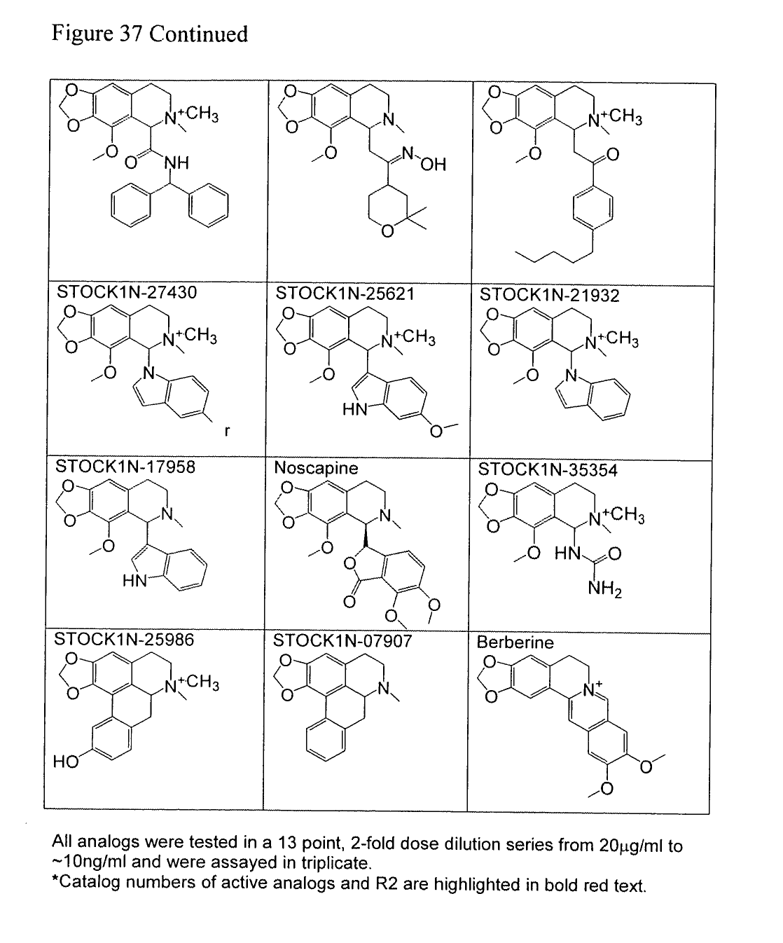

Additional examples of compounds of Formula XIV are exemplified in FIG. 37.

### Pharmaceutical Compositions

A compound of the present invention, such as the compounds described above and/or in FIGS. 5-6, 16-20, 24, and 36-37 may be administered to an individual in need thereof. In certain embodiments, the individual is a mammal such as a human. When administered to an individual, the compound of the invention can be administered as a pharmaceutical composition (preparation) containing, for example, the compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, and oils such as olive oil or injectable organic esters.

A pharmaceutically acceptable carrier can contain physiologically acceptable agents that act, for example, to stabilize or to increase the absorption of a subject compound such as a tubulin inhibitor. Such physiologically acceptable agents include, for example, carbohydrates, such as glucose, sucrose or dextrans, antioxidants, such as ascorbic acid or glutathione, chelating agents, low molecular weight proteins, or other stabilizers or excipients. The choice of a pharmaceutically acceptable carrier, including a physiologically acceptable agent, depends, for example, on the route of administration of the composition. The pharmaceutical composition (preparation) also can be a liposome or a solid (e.g., polymer) matrix (e.g., in a tablet or sustained release implant), which can have incorporated therein, for example, a compound of the invention. Liposomes, for example, which consist of phospholipids or other lipids, are nontoxic, physiologically acceptable and metabolizable carriers that are relatively simple to make and administer.

A pharmaceutical composition (preparation) containing a compound of the invention can be administered to a subject in need thereof by any of a number of routes of administration including, for example, orally; intramuscularly; intravenously; anally; vaginally; parenterally; nasally; intraperitoneally; subcutaneously; intrathecally; by inhalation; or topically.

In certain embodiments, the compound of the present invention may be used alone or conjointly administered with another type of therapeutic agents for treating neurodegenerative disorders (e.g., HD). As used herein, the phrase “conjoint administration” refers to any form of administration in combination of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. Thus, an individual who receives such treatment can have a combined (conjoint) effect of different therapeutic compounds.

It is contemplated that the compound of the present invention will be administered to a subject (e.g., a mammal, preferably a human) in a therapeutically effective amount (dose). By “therapeutically effective amount” as used herein is meant to be the concentration of a compound that is sufficient to elicit the desired therapeutic effect (e.g., inhibition of neuronal cell death). It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. Typically, for a human subject, an effective amount will range from about 0.001 mg/kg of body weight to about 30 mg/kg of body weight, or more generally between 10 mg and 1,000 mg, or between 50 mg and 500 mg, or between 100 mg and 500 mg. A larger total dose can be delivered by multiple administrations of the agent. Methods to determine efficacy and dosage are known to those skilled in the art. (See, for example, Isselbacher et al. (1996) Harrison's Principles of Internal Medicine 13 ed., 1814-1882, herein incorporated by reference). In specific, non-limiting embodiments, the pharmaceutical composition is sterile or sterilized.

The present invention has determined that these experimentally engineered cells (e.g., neuronal cells) make it possible to identify genotype-selective agents from both known and novel compound sources that suppress toxicity to or promote viability of cells in the presence of specific alleles (e.g., a mutant huntingtin gene).

In certain aspects, the present invention relates to the development of high-throughput screens for suppressors (e.g., small molecules) of the toxicity of expanded huntingtin (eHtt) in neuronal cells. A collection of compounds were screened in these assays and compounds were identified that promote viability of neuronal cells expressing a mutant expanded huntingtin, but not of neuronal cells lacking mutant expanded huntingtin. These identified genotype-selective compounds may serve as molecular probes of signaling networks present in neuronal cells from HD patients, and as leads for subsequent development of clinically effective drugs with a favorable therapeutic index.

### Methods of Treatment

In certain embodiments, the invention provides methods for treating or preventing a neurodegenerative disorder associated with polyglutamine (polyQ) expansion, in an individual in need thereof. Such can be accomplished by inhibiting neuronal cell death or degeneration, for example of a neuronal cell at risk for death or degeneration due to a genetic disorder associated with polyQ expansion, by administering an effective amount of a compound of the invention.

In one embodiment of the invention, the method comprises administering to the individual a therapeutically effective amount of an agent identified by the methods of the invention (e.g., a compound shown in FIGS. 5-6, 16-20, 24, and 36-37). As described herein, the neurodegenerative disorders associated with polyQ expansion include, but are not limited to, Huntington's disease, spinobulbar muscular atrophy, dentatorubral pallidoluysian atrophy, and the spinocerebellar ataxias type 1, 2, 3, 6, 7, and 17. For example, a tubulin inhibitor can be administrated to an individual suffering from HD or at risk of having HD, for therapeutic or prophylactic purposes.

In specific non-limiting embodiments, the present invention provides for methods of treating a neurodegenerative disorder selected from the group consisting of Huntington's Disease, spinobulbar muscular atrophy, dentatorubral pallidoluysian atrophy, and the spinocerebellar ataxias type 1, 2, 3, 6, 7, and 17, comprising administering an agent depicted in FIG. 5, 6, 16, 17, 18, 19, 20, 24 and 36-37, in an effective amount, for example to achieve a local concentration in the brain of the subject of between about 1 and 50 μg/ml, preferably between about 1 and 20 μg/ml, or between about 5 and 10 μg/ml. In certain non-limiting embodiments, an agent according to the invention may be used in methods to inhibit neuronal cell death, or enhance neuronal survival, or achieve both effects.

In another embodiment, the present invention contemplates methods of treating or preventing a neurodegenerative disorder (e.g., HD) by modulating the function (e.g., activity or expression) of a cellular component that is identified according to the invention. To illustrate, if a cellular component is identified to promote polyglutamine-mediated neurotoxicity, a therapeutic agent can be used to inhibit or reduce the function (activity or expression) of the cellular component. Alternatively, if a target is identified to inhibit polyglutamine-mediated neurotoxicity, a therapeutic agent can be used to enhance the function (activity or expression) of the cellular component. The therapeutic agent includes, but is not limited to, an antibody, a nucleic acid (e.g., an antisense oligonucleotide or a small inhibitory RNA for RNA interference), a protein, a small molecule or a peptidomimetic.

### Methods of Screening

In certain embodiments, the invention relates to a method of identifying agents (drugs) that selectively suppresses the cellular toxicity in engineered cells, for example, engineered neuronal cells expressing a mutant expanded huntingtin protein. In one embodiment, the invention relates to a method of identifying an agent (drug) that suppresses the cellular toxicity of a mutant expanded huntingtin protein in engineered cells, comprising contacting test cells (e.g., engineered neuronal cells expressing a mutant expanded huntingtin protein) with a candidate agent; determining viability of the test cells contacted with the candidate agent; and comparing the viability of the test cells with the viability of an appropriate control. If the viability of the test cells is more than that of the control cells, then an agent (drug) that selectively suppresses the cellular toxicity (e.g., expanded huntingtin-induced cellular toxicity) is identified. An appropriate control is a cell that is the same type of cell as that of test cells except that the control cell is not engineered to express a protein which causes toxicity. For example, control cells may be the parental primary cells from which the test cells are derived. Control cells are contacted with the candidate agent under the same conditions as the test cells. An appropriate control may be run simultaneously, or it may be pre-established (e.g., a pre-established standard or reference).

As used herein, the term “toxicity” refers to the ability of an agent, such as a polyQ expanded mutant htt protein, to kill or inhibit the growth/proliferation of cells. The term “toxicity-suppressing activity” refers to the ability of a molecule to inhibit or decrease the toxicity to cells caused by an agent (e.g., a polyQ expanded mutant htt protein), thereby promoting cell viability (growth or proliferation). Large-scale screens include screens wherein hundreds or thousands of compounds are screened in a high-throughput format for selective toxicity-suppressing activity in neuronal cells.

In certain embodiments, the present invention relates to engineered neuronal cell lines, for example, neuronal cells engineered to express a mutant expanded huntingtin (htt) protein. Non-limiting examples of these neuronal cells include rat neuronal PC12 cells and rat striatal neuronal ST14A cells as described in the Examples below. To illustrate, PC12 cells or ST14A cells can be transfected with exon-1 of the human expanded huntingtin gene containing expanded polyQ repeats (e.g., Q103) at the N-terminal region. Expressing polyQ-expanded human expanded huntingtin exon-1 (Htt-Q103) in these cells can lead to selective toxicity over wild-type (e.g., Htt-Q25) expressing cells.

The normal function of htt and the mechanism of toxicity caused by expanded polyQ stretches are still unclear. Both a gain of novel function and a loss of normal function have been proposed to explain pathology caused by polyQ expansions in htt. The htt protein is largely cytoplasmic and is associated to some extent with microtubules (MT) and membranous compartments of the cell. Diverse functions have been proposed for htt because of its interactions with proteins involved in cellular transport (HAP1), cell death (HIPPI), transcription machinery (CBP, TAFII130) and metabolism (GAPDH). Also, cell toxicity shows context dependence since the extreme N-terminal fragments containing the glutamine repeats are more toxic than larger fragments or full length Htt.

In certain embodiments, the candidate agent is selected from a compound library, such as a combinatorial library. Cell viability may be determined by any of a variety of means known in the art, including the use of dyes such as calcein acetoxymethyl ester (calcein AM) and Alamar Blue. In certain embodiments of the invention, a dye such as calcein AM is applied to test and control cells after treatment with a candidate agent. In live cells, calcein AM is cleaved by intracellular esterases, forming the anionic fluorescent derivative calcein, which cannot diffuse out of live cells. Hence, live cells exhibit a green fluorescence when incubated with calcein AM, whereas dead cells do not. The green fluorescence that is exhibited by live cells can be detected and can thereby provide a measurement of cell viability.

In certain embodiments of the invention, an agent that has been identified as one that selectively suppresses toxicity to neuronal cells is further characterized in an animal model. Animal models include mice, rats, rabbits, and monkeys, which can be nontransgenic (e.g., wildtype) or transgenic animals. The effect of the agent that selectively suppresses toxicity to neuronal cells may be assessed in an animal model for any number of effects, such as its ability to selectively promote neuronal cell viability or growth in the animal.

### Methods of Identifying Targets for Neuroprotective Compounds

In certain embodiments, the invention relates to the use of the subject genotype-selective compound, also referred to herein as “ligand” (e.g., a compound shown in FIGS. 5-6, 16-20, and 24), to identify targets (also referred to herein as “cellular components” (e.g., proteins, nucleic acids, or lipids) involved in conferring the phenotype of diseased cells.

In one embodiment, the invention provides a method to identify cellular components involved in polyglutamine-mediated neurotoxicity, whereby a neuronal cell, such as an engineered neuronal cell, is contacted with a subject compound; and after contact, cellular components that interact (directly or indirectly) with the compound are identified, resulting in identification of cellular components involved in polyglutamine-mediated neurotoxicity.

In a specific embodiment, the invention provides a method to identify cellular components involved in HD, whereby a cell having huntingtin-induced toxicity, such as an engineered neuronal cell, is contacted with an anti-HD test compound. After contact, cellular components that interact (directly or indirectly) with the anti-HD test compound are identified, resulting in identification of cellular components involved in HD.

As described herein, the subject compound (or ligand) of these methods may be created by any combinatorial chemical method. Alternatively, the subject compound may be a naturally occurring biomolecule synthesized in vivo or in vitro. The ligand may be optionally derivatized with another compound. One advantage of this modification is that the derivatizing compound may be used to facilitate ligand target complex collection or ligand collection, e.g., after separation of ligand and target. Non-limiting examples of derivatizing groups include biotin, fluorescein, digoxygenin, green fluorescent protein, isotopes, polyhistidine, magnetic beads, glutathione S transferase, photoactivatible crosslinkers or any combinations thereof.

According to the present invention, a target (cellular component) may be a naturally occurring biomolecule synthesized in vivo or in vitro. A target may be comprised of amino acids, nucleic acids, sugars, lipids, natural products or any combinations thereof. An advantage of the instant invention is that no prior knowledge of the identity or function of the target is necessary.

The interaction between the ligand and target may be covalent or non-covalent. Optionally, the ligand of a ligand-target pair may or may not display affinity for other targets. The target of a ligand-target pair may or may not display affinity for other ligands.

For example, binding between a ligand and a target can be identified at the protein level using in vitro biochemical methods, including photo-crosslinking, radiolabeled ligand binding, and affinity chromatography (Jakoby et al., Methods in Enzymology 1974, 46: 1). Alternatively, small molecules can be immobilized on an agarose matrix and used to screen extracts of a variety of cell types and organisms.

Expression cloning can be used to test for the target within a small pool of proteins (King et. al., Science 1997, 277:973). Peptides (Kieffer et. al., PNAS 1992, 89:12048), nucleoside derivatives (Haushalter et. al., Curr. Biol. 1999, 9:174), and drug-bovine serum albumin (drug-BSA) conjugate (Tanaka et. al., Mol. Pharmacol. 1999, 55:356) have been used in expression cloning.

Another useful technique to closely associate ligand binding with DNA encoding the target is phage display. In phage display, which has been predominantly used in the monoclonal antibody field, peptide or protein libraries are created on the viral surface and screened for activity (Smith G P, Science 1985, 228:1315). Phages are panned for the target which is connected to a solid phase (Parmley et al., Gene 1988, 73:305). One of the advantages of phage display is that the cDNA is in the phage and thus no separate cloning step is required.

A non-limiting example includes binding reaction conditions where the ligand comprises a marker such as biotin, fluorescein, digoxygenin, green fluorescent protein, radioisotope, histidine tag, a magnetic bead, an enzyme or combinations thereof. In one embodiment of the invention, the targets may be screened in a mechanism based assay, such as an assay to detect ligands which bind to the target. This may include a solid phase or fluid phase binding event with either the ligand, the protein or an indicator of either being detected. Alternatively, the gene encoding the protein with previously undefined function can be transfected with a reporter system (e.g., β-galactosidase, luciferase, or green fluorescent protein) into a cell and screened against the library preferably by a high throughput screening or with individual members of the library. Other mechanism based binding assays may be used, for example, biochemical assays measuring an effect on enzymatic activity, cell based assays in which the target and a reporter system (e.g., luciferase or β-galactosidase) have been introduced into a cell, and binding assays which detect changes in free energy. Binding assays can be performed with the target fixed to a well, bead or chip or captured by an immobilized antibody or resolved by capillary electrophoresis. The bound ligands may be detected usually using colorimetric or fluorescence or surface plasmon resonance.

### EXEMPLIFICATION

The invention now being generally described, it will be more readily understood by reference to the following examples, which are included merely for purposes of illustration of certain aspects and embodiments of the present invention, and are not intended to limit the invention.

### Example 1

### Screens for Small Molecule Suppressors of Expanded Huntingtin in Mammalian Cells

There are nine inherited neurodegenerative disorders caused by a polyglutamine (polyQ)-encoding trinucleotide (CAG) repeat expansion within the coding sequence of a gene. These diseases include Huntington's Disease, spinobulbar muscular atrophy, dentatorubral pallidoluysian atrophy, and the spinocerebellar ataxias type 1, 2, 3, 6, 7, and 17. Precisely how polyQ mutations lead to neuronal loss in each disease remains unclear; however, several molecular characteristics appear to be shared among the different disorders. Such characteristics include deficiencies in ubiquitin-mediated proteolysis, protease-dependent accumulation of polyQ protein fragments, formation of cytosolic and nuclear inclusions, and changes in gene expression (Zoghbi H Y and Orr H T, Annu Rev Neurosci 2000, 23: 217-47; Kaytor M D & Warren S T, J Biol Chem 1999, 274: 37507-10; Orr H T, Genes Dev 2001, 15: 925-32; Taylor J P, et al., Science 2002, 296: 1991-5; Rubinsztein D C, Trends Genet 2002, 18: 202-9).

The slow, progressive characteristic of Huntington's Disease (HD) makes it difficult to study in humans, although postmortem brain analysis of HD patients has been useful in revealing extensive neuronal loss in regions of the brain functionally affected during the course of the disease (Gutekunst C A, et al., J Neurosci 1999, 19: 2522-34). Although the huntingtin protein is expressed in many cell types, there is a relatively selective disappearance of medium spiny neurons in the striatum of patients with HD. Cell-based models that recapitulate aspects of this cell-type specific death are of value (Schweitzer E S, et al., submitted).

The present Example describes the development of two high-throughput, neuronal cell-based screens related to Huntington's Disease. Both assays exhibit mutant huntingtin-dependent toxicity that is found selectively in neuronal cells. These screens allow identification of small molecules that prevent the toxicity of the expanded, polyglutamine-containing huntingtin protein in neuron-like cells in culture.

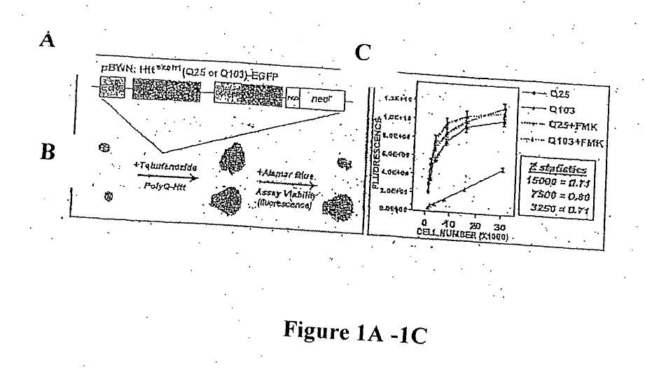

In the first cell system developed in collaboration with Dr. Erik Schweitzer (UCLA), a high-throughput screen (HTS) for compounds that rescue polyQ-induced apoptosis in immortalized rat neuronal cells (Suhr S T, et al., Proc Natl Acad Sci USA 1998, 95: 7999-8004). PC12 rat pheochromocytoma cells were transfected with exon-1 of the human huntingtin gene containing either 25 or 103 N-terminal polyQ repeats. For enhanced stability the repeat portion consists of alternating CAG/CAA repeats (FIG. 1A). In addition, the expression construct incorporates enhanced green fluorescent protein (EGFP) as a reporter that enables tracking of the fusion proteins by direct immunofluorescence microscopy or biochemical (immunoprecipitation or Western blotting) detection with anti-EGFP antibodies (Schweitzer E S, et al., submitted). Finally, expression is regulated using the Bombyx mori ecdysone receptor and ecdysone analog, tebufenozide (Suhr S T, et al., Proc Natl Acad Sci USA 1998, 95: 7999-8004).

Following induction with tebufenozide, these cells express comparable levels of either mutant or non-mutant forms of huntingtin. Mutant huntingtin (Q103)-expressing, but not wild-type huntingtin (Q25)-expressing, cells display perinuclear cytoplasmic inclusions (CIs) and begin to die 24 hours after induction of expression (FIG. 1C). Expression of the Q103 construct in an astrocyte-like cell line (BAS 8.1) did not result in perinuclear aggesome formation or cytotoxicity, demonstrating that toxicity of Htt in this model is cell-type-specific.

In the second cell system, a high-throughput screen was developed in collaboration with Elena Cattaneo (University of Milano, Italy) using embryonic rat striatal neuronal cells immortalized with a temperature-sensitive SV40 Large T antigen (ST14A cells). These ST14A cells have been engineered to express constitutively either an N-terminal 548 amino acid fragment of the human huntingtin protein (wt) or the pathogenic version containing an expanded polyglutamine (mutant). Both of these cell lines proliferate normally at the permissive temperature (33° C.) but upon a shift to the non-permissive temperature (39° C.), T antigen is degraded and the cells differentiate into striatal neuronal cells (Ehrlich M E, et al., Exp Neurol 2001, 167: 215-26; Rigamonti D, et al., J Neurosci 2000, 20: 3705-13; Weinelt S, et al., J Neurosci Res 2003, 71: 228-36; Torchiana E, et al., Neuroreport 1998, 9: 3823-7; Cattaneo E & Conti L, J Neurosci Res 1998, 53: 223-34; Cattaneo E, et al., J Biol Chem 1996, 271: 23374-9; Corti O, et al., Neuroreport 1996, 7:1655-9). These differentiated cells are sensitive to the toxic effects of mutant huntingtin and die at an enhanced rate compared to the wt huntingtin-expressing cells.

1. PC12 Assay System

A) Assay Development

The high-throughput screen using the PC12 cell system uses the fluorescent viability dye Alamar Blue™ (FIG. 1B). Using this assay, up to a five-fold decrease in viability of the Htt-Q103 cells was detected as compared to the control Htt-Q25 cells (FIG. 1C). One important parameter in cell-based HTS to be optimized is the cell number per well that yields the best separation between the positive and negative signal (in this case, viable versus dead cells). The Z-factor is a commonly used quantitative index for maximal signal separation and minimal variability. A Z-factor greater than 0.2 is typically required for robust screening results (the theoretical range is from −∞ to 1, with 1 being maximal) (Zhang J H, et al., J Biomol Screen 1999, 4: 67-73). A plot of the mean (N=8) fluorescence (viability) versus a variety of cell numbers revealed that the maximal statistical separation (Z statistic=0.8) occurred at 7500 cells per well.

Caspase inhibitors have been reported to rescue polyQ-mediated toxicity in several systems, including the one described here (Chen M, et al., Nat Med 2000, 6: 797-801; Kim M, et al., J Neurosci 1999, 19: 964-73; Rigamonti D, et al., J Biol Chem 2001, 276: 14545-8; Wellington C L & Hayden M R, Clin Genet 2000, 57: 1-10; Ellerby L M, et al., J Neurochem 1999, 72: 185-95). As a control, the ability of the general caspase inhibitor BOC-D-FMK to rescue Htt-Q103-mediated cell death in this assay system was tested. The addition of 50 μM BOC-D-FMK to Htt-Q103 cells at the time of tebufenozide induction resulted in a complete (100+%) rescue of the Htt-Q103-induced cytotoxicity (FIG. 1C).

B) Primary Screening

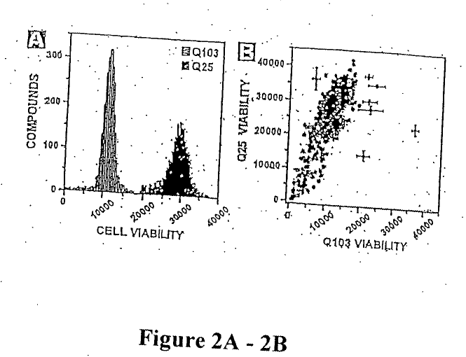

Having defined HTS parameters for the PC12 cell system, approximately 2,500 biologically active compounds were screened from a collection previously assembled. The primary screen of these compounds was performed in triplicate at a concentration of 4 μg/ml (˜10 μM) with 0.1% dimethyl sulfoxide. The procedure for library screening of the PC12 cells consisted of the following: (1) seed cells into 384-well plates with complete medium containing inducing compound (e.g., tebufenozide); (2) transfer library compounds from freshly generated daughter plates to cell culture plates with an integrated Zymark Sciclone/Twister II robot; (3) incubate culture plates for 72 hours (37° C., 9.5% CO₂ for PC12 cells); and (4) add viability dye (Alamar Blue™), incubate for an additional 12-16 hours, and read plates in a fluorescence plate reader (Packard integrated minitrak/sidetrak/Fusion). Dilution and detection of Alamar Blue™ was performed as recommended by the manufacturer (Biosource International). The results of the primary screen of this library are shown in FIG. 2. This screen revealed several compounds that specifically suppressed Q103-induced toxicity and one compound that operates as an enhancer. These six selective suppressors (FIG. 2B) are not known to function as general death suppressing agents (e.g., as caspase inhibitors).

C) Secondary Screening

Compounds selected as being drawn from a distribution different from that of the vehicle-treated cells in the primary screen (p<0.05) were retested in an 11-point, two-fold dilution series in four replicates to confirm activity and to determine the dose response. The dilution curves were created robotically using custom-generated software for the Sciclone and Twister II. All other assay conditions for the secondary screen were identical to that of the primary screen with the exception of compound concentration.

2. ST14A Assay System

A) Assay Development

In this Example, a fluorescence viability assay was used to monitor cell death in ST14A-Htt^{wt} and ST14A-Htt^{mut} cell lines. The assay is based on conversion of a non-fluorescent substrate (calcein AM, Molecular Probes, Eugene, Oreg.) to a fluorescent product by nonspecific esterases in live cells. Thus, cell death is indicated by a decrease in fluorescence. Cells were seeded in 384-well plates in DMEM medium with 0.1 mM sodium pyruvate and 2 mM glutamine with different amounts of serum. The plates were incubated at 33° C. for 3 h and then shifted to 39° C. (with 5% CO₂) and incubated for various time intervals (see below). The wells were washed in phosphate buffered saline ten times, incubated with calcein AM for 4 h and fluorescence was recorded with a read time of 0.2 seconds per well on a fluorescence platereader (Packard Fusion).

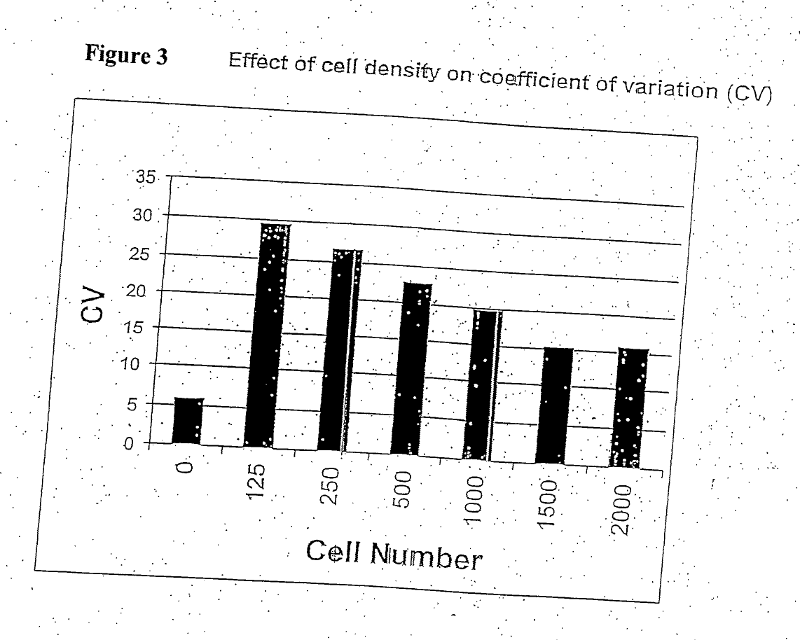

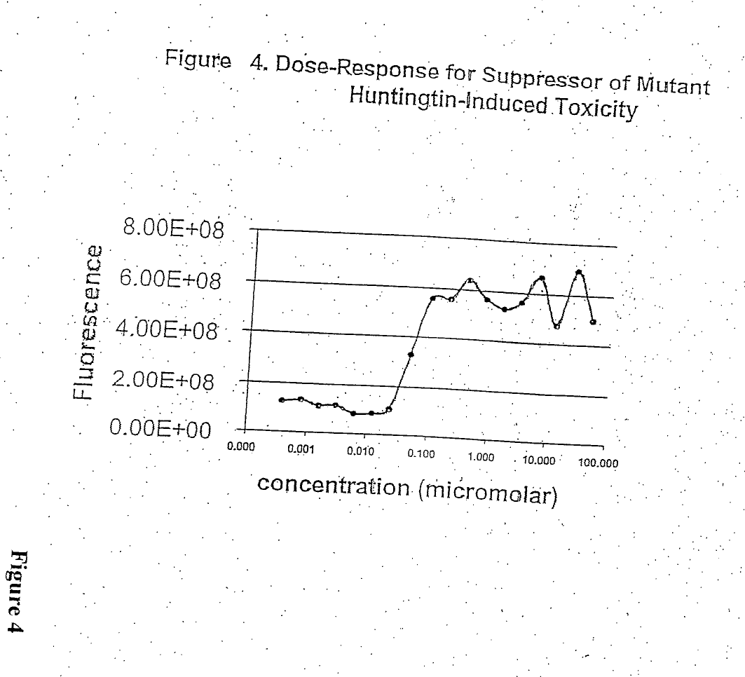

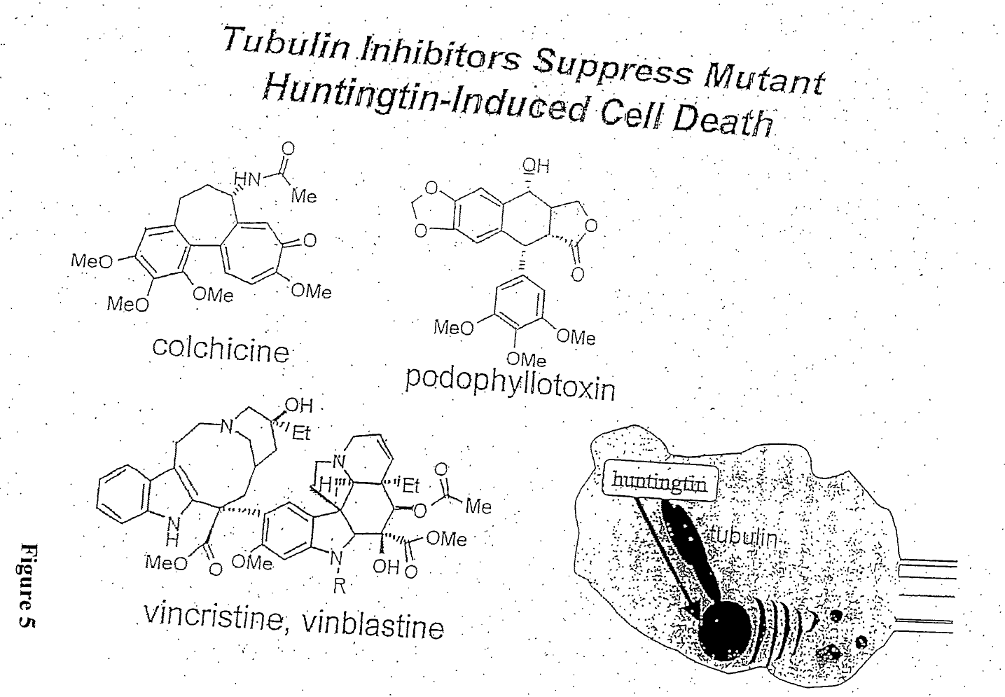

The range of cell numbers that gave a linear increase in fluorescence were tested. The signal was linear over a range of 125-1500 cells per well and was saturated above 2000 cells per well. The coefficient of variation as a percentage of signal (% CV) was high (30-40%) at low cell density and decreased to 15-20% with 1500 or more cells per well (FIG. 3). The duration of calcein incubation was four hours, as the signal did not saturate with up to five hours of incubation of cells with calcein AM at room temperature. The percentage of serum was titrated to 0.5% inactivated fetal calf serum (Sigma) to enhance cell death such that the average fluorescence of live cells on the day of plating was 2-3 fold higher than cells after three days at 39° C. in 0.5% serum. The Z factor was consistently between 0.1 and 0.25 under these conditions. Although the Z factor is marginal in this assay, it was found to be sufficient when triplicate measurements are used, as is a standard practice.

Using the optimized assay, the 2,500 bioactive compound library was screened for inhibitors of mutant huntingtin-induced death of ST14A cells. The library was screened twice, with triplicate tests of each compound performed in each screen. The cutoff for a hit was arbitrarily defined as a 1.5-fold increase in signal in comparison to the average fluorescence on the plate in at least two of the three wells of triplicate testing.

All hits that appeared in the two independent screens of a library were compiled and these potential hits were tested for activity in a dose-titration assay (FIG. 4). Compounds that appeared as positives in the dose-titration assay were reordered from a commercial supplier and were retested again in a dose titration. All compounds that showed activity under these conditions were selected as hits. These compounds were tested for activity in the mutant, wild type and parent cell lines. These selectivity data are indicated in Table 1.

TABLE 1
Selectivity of suppressors in ST14A cell lines

| | Mutant | Wild type | Parent |
|---|---|---|---|
| 1 | + | + | − |
| 2 | + | + | − |
| 3 | + | + | − |
| 4 | + | + | − |
| 5 | + | − | + |
| 6 | + | − | − |
| 7 | + | − | − |
| 8 | + | + | + |
| 9 | + | − | − |
| 10 | + | + | − |
| 11 | + | + | − |
| 12 | + | − | − |

Twelve suppressors of mutant huntingtin-induced death were identified (out of ˜2,500 tested) in the ST14A cell system. These compounds were tested in six replicates in dilution series in mutant huntingtin-expressing cells, wild-type huntingtin-expressing cells and the parental ST14A cells lacking any construct. Four categories of compounds were identified. First, compounds that increase viability of all three cell types. Second, compounds that increase viability of mutant and wild-type huntingtin-expressing cells but not of the parental ST14A cells. Third, compounds that increase viability of both the mutant and parental cells but not the wild-type cells. Fourth, compounds that increase viability only of the mutant cells.

### Example 2

### Identification of Small Molecule Suppressors of Polyglutamine Neurotoxicity

Huntington's Disease (HD) is one of at least nine inherited neurological disorders caused by trinucleotide (CAG) repeat expansion (others being Kennedy's disease, dentatorubro-pallidoluysian atrophy, and six forms of spinocerebellar ataxia). One aim of these experiments is to identify small molecule suppressors of PolyQ neurotoxicity and to elucidate mechanisms of polyQ neurotoxicity through studying the functional means by which the identified compounds suppress polyQ-expanded Htt toxicity.

1. Identification of Compounds that Suppress PolyQ-Htt Toxicity in PC12 Cells

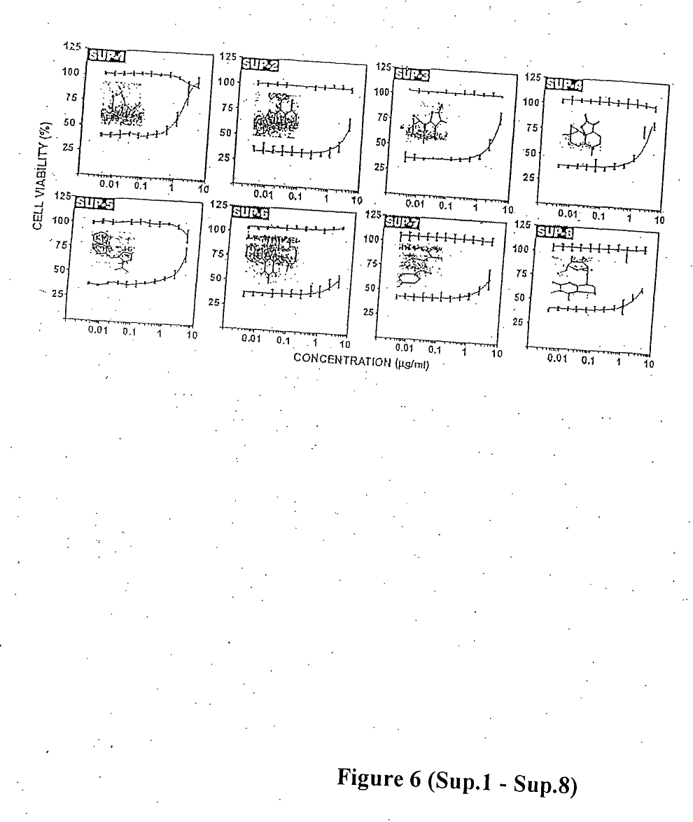

As described in Example 1, it was found that expressing polyQ-expanded human huntingtin exon-1 (Htt-Q103) in rat neuronal (PC12) cells led to selective toxicity over wild-type (Htt-Q25) expressing cells. Using this PC12 model assay system, approximately 50,000 small molecules (MW<2000 Daltons) were screened for their ability to prevent polyQ-mediated toxicity. 8 compounds (referred to herein as SUP-1 to SUP-8) were identified that specifically inhibit Q103-induced cytotoxicity, four of which restore viability to 80% of wild-type treated cells (FIG. 6).

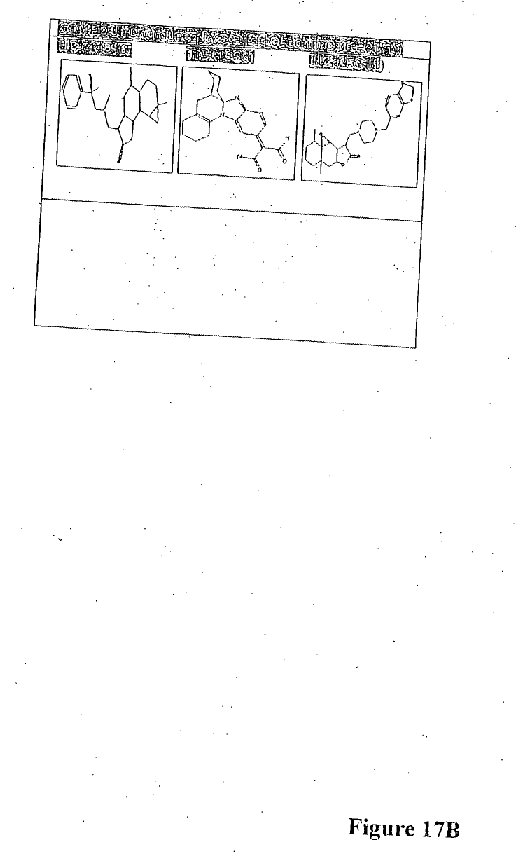

Using the PC12 cell assay system, additional compounds and analogs which are able to prevent polyQ-mediated toxicity were identiifed (see FIG. 16). A summary of certain representative compounds (analogs) is shown in FIG. 17.

2. Characterization of the Mechanism of Action (MOA) for Small Molecule Suppressors of PolyQ-Expanded Htt Protein Toxicity

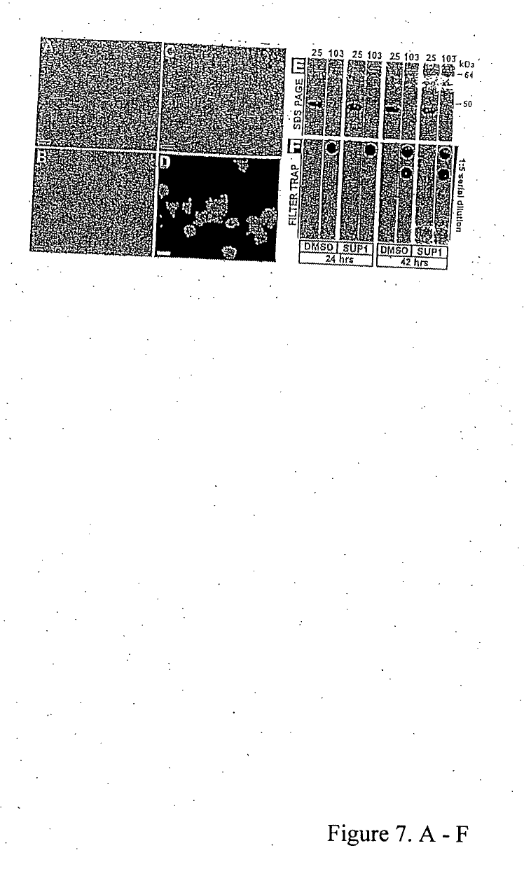

The MOA for the eight suppressors shown in FIG. 6 were characterized. The initial characterization criteria included assessment of the following: (1) ability to restore “normal” cell morphology to Htt-Q103 expressing cells; (2) eliminating compounds that were general suppressors of Htt-Q25/103 protein expression; and (3) examining whether any of the suppressors altered Htt-Q103 aggregate formation. Of the eight suppressors, SUP-1-4 were the best at restoring Htt-Q103 expressing cells to a “normal” cell morphology (e.g., uninduced or Htt-Q25-like morphology, FIG. 7). None of the suppressors appeared to function by down-regulating Htt-Q103 protein expression and none were able to significantly alter Htt-Q103 aggregate formation (results for SUP-1are shown in FIG. 7, as a representative of these experiments).

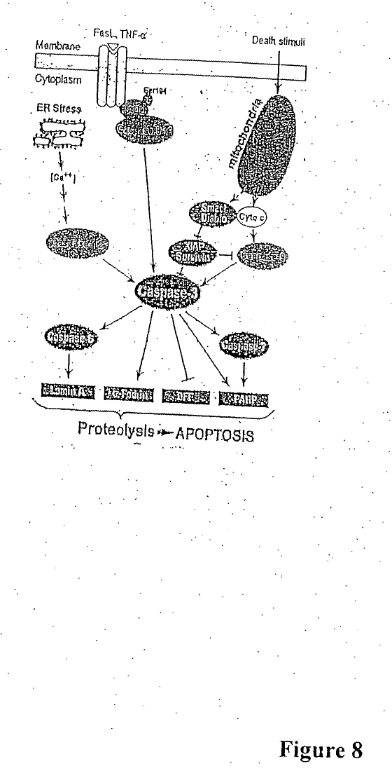

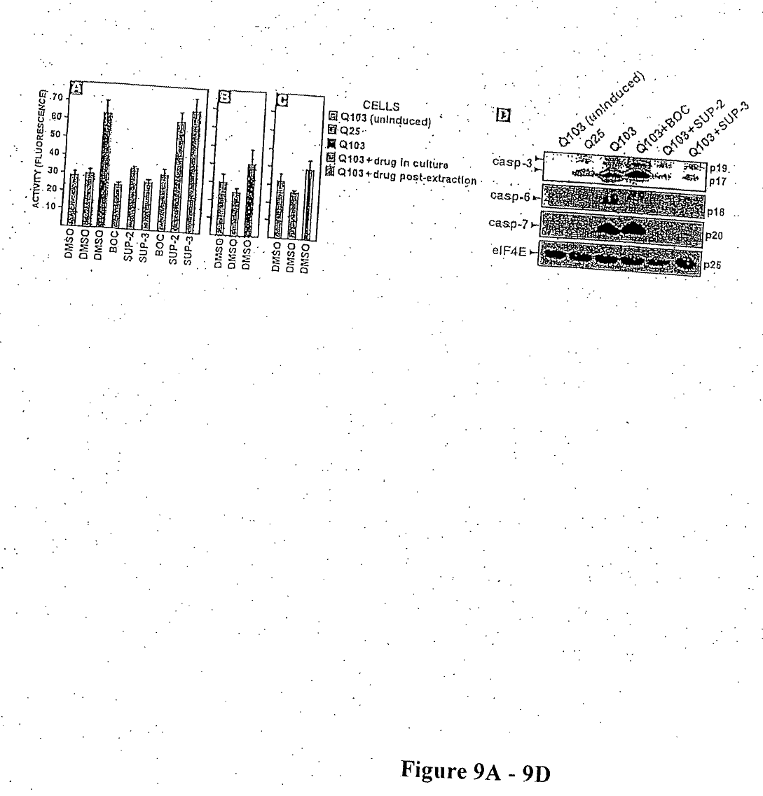

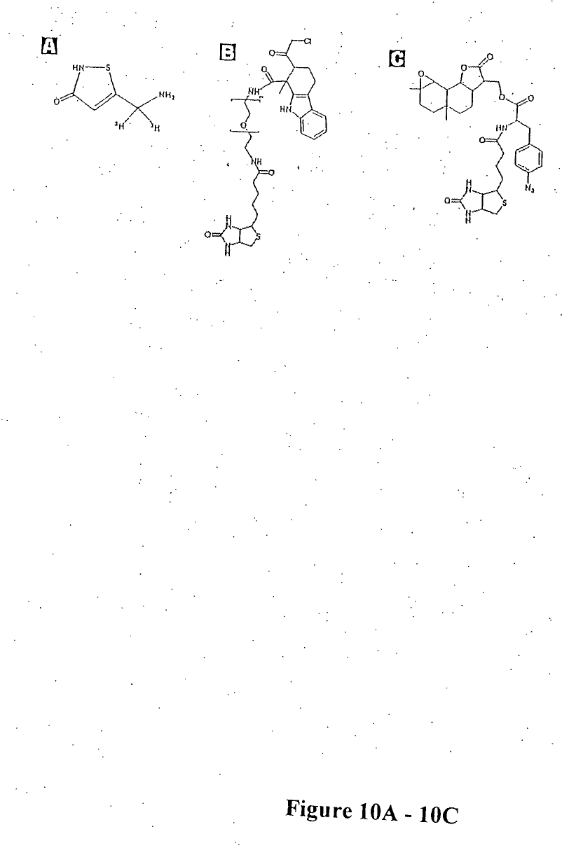

A fourth level of preliminary MOA characterization was to assess whether any of the compounds function as general death suppressors. To assay for general death suppression, all of the suppressors were tested for their ability to rescue apoptosis induced by serum starvation. Interestingly, the top four suppressors were able to suppress serum-starved induced apoptosis of untransfected PC12 cells (viability assessed via Alamar Blue). Caspase activation is central to both serum-starved and poly-Q-mediated apoptosis (FIG. 8). To further characterize the anti-apoptotic MOA of the top four suppressors, the ability to alter caspase activation was examined. Following a 15-hour induction period with tebufenozide, Htt-Q103 cells showed elevated levels of caspase-3 activity over uninduced Htt-Q103 or induced Htt-Q25 expressing cells (FIG. 9). When added to the cells in culture, SUP-2 and SUP-3 suppressed caspase-3 activation. Interestingly, unlike the general caspase inhibitor, BOC-D-FMK (BOC), SUP-2 and SUP-3 did not directly inhibit caspase-3 activity in solution, suggesting they function upstream of caspase-3 cleavage (FIG. 9). Furthermore, Western blot analysis for active caspase 3, 6, and 7 cleavage products under the same experimental conditions also show SUP-2 and SUP-3 to be acting upstream of effector caspase activation (FIG. 9).

To further characterize MOA of the suppressors identified in the primary screen, the following studies were proposed, including characterizing suppressor-induced changes in: (1) apoptotic signaling proteins (e.g., caspases and IAP's), and (2) proteolytic processing of Htt-Q103 cleavage products (fragments) using basic biochemical techniques.

PolyQ-containing proteins have been shown to induce apoptosis through both caspase dependent (caspase-8, 9, and 12) and independent pathways. Preliminary data shows only a modest activation of initiator caspases-8 and 9 in Htt-Q103 cells suggesting alternative pathways of activation (FIG. 9). Caspase-12 stands out as a strong initiator candidate and has been shown to be activated by polyQ-induced ER stress. Active caspase-12 specific antibodies are currently not commercially available. However, fluorometric caspase-12 substrates can be custom synthesized for about the same cost as antibodies (Molecular Probes, Eugene, Oreg.). In addition, it would be of interest to test top suppressors for modulation and inhibition of specific initiator caspases in both mouse and human neuronal cell lines. Organelle specific apoptosis-inducing drugs such as Brefeldin-A, etoposide, and staurosporine will help to confirm specific caspase pathways that are altered or suppressed by specific compounds (Wang et al., 2003, Proc Natl Acad Sci USA, 100:10483-10487; Duan, et al., 2003, J Biol Chem, 278:1346-1353; Robertson et al., 2002, J Biol Chem, 277:29803-29809; Guo et al., 1998, Exp Cell Res, 245:57-68). Finally, proteolytic (caspase-mediated) cleavage of polyQ-expanded Htt protein has been proposed mechanism of polyQ-induced toxicity. Detailed biochemical analysis, for example silver staining of immunoprecipitated Htt protein from compound treated cells, may reveal changes in proteolysis that are central to polyQ-mediated toxicity (Wellington et al., 2000, J Biol Chem, 275:19831-19838; Wellington et al., 2002, J Neurosci, 22:7862-7872).

3. Identification and Validation of Suppressor Molecule Target Proteins.

Target proteins will be identified using either biotinylated or tritiated compound analogs. Target proteins will be isolated by gel (SDS-PAGE) or affinity purification (avidin coupled agarose) and sequenced using tandem mass spectrometry (Gygi Lab, Taplin Biological Mass Spec Facility, Harvard Medical School). Target validation will be performed via siRNA knockdown of the identified protein (Hannon et al., 2002, Nature 418:244-251; Tuschl et al., 2002, Nat Biotechno 120:446-448; Dolma et al., 2003, Cancer Cell 3:285-296).

A) SUP-1 Target Identification

Thiomuscimol (SUP-1) is known to function as a GABA_{A} receptor agonist. Its ability to suppress Htt-Q103 toxicity, however, does not appear to be through this mechanism since other GABA receptor agonists (40 total from the primary screen, including structurally related compounds muscimol and THIP) were not active. The synthesis of thiomuscimol and a tritiated form of the compound have been published (Frolund et al., 1995, Compounds and Radiopharmaceuticals 35:877-889). In addition, it has been shown that thiomuscimol can be covalently coupled to interacting proteins (e.g., GABA_{A} receptor) by photo-crosslinking (Nielsen et al., 1995, European J Pharmacology Molecular Pharmacology Section 289:109-112). These methods will be used to try and identify the SUP-1 target protein and determine its biological MOA.

B) SUP-2 Target Identification

SUP-2 and SUP-7 both contain chloromethyl ketone groups which are known to be functionally active groups in caspase inhibitors such as z-VAD-FMK and BOC-D-FMK. Thus, the ability of these molecules to suppress effector caspase activation is likely the result of covalent binding and subsequent inactivation of a protease upstream of caspase-3 (FIG. 8). Activity studies of SUP-2 analogs suggest modifying the compound to incorporate a biotinylated handle as depicted in FIG. 10 should not alter compound activity. Similar modifications have been used to successfully isolate small molecule target proteins.

C) SUP-3 (and analog SUP-4) Target Identification

Suppressors SUP-3 and SUP-4 (and additional active analogs not shown) do not contain chloromethyl ketone groups as noted above for SUP-2. Activity studies of SUP-3 analogs suggest that reduction of the exocyclic olefin to contain a biotinylated handle should not alter the compounds activity. It is also likely that these compounds are forming a covalent linkage with their target through the lactone or epoxide groups. Alternatively, photoactivatable cross-linkers can be incorporated into biotinylated analogs and used to covalently couple small molecule suppressors to their targets (Dorman et al., 2000, TIBTECH 18:64-76; Fancy and Kodadek, 1999, Proc Natl Acad Sci USA 96:6020-6024; Weber et al., 1997, J Peptide Research, 375-383; and FIG. 10).

In addition to selecting compounds for future development as potential therapeutics to treat polyglutamine disease, these studies will yield powerful tools to reveal mechanisms that ultimately lead to polyQ-induced apoptosis. Furthermore, identifying compound targets may lead to the characterization new apoptotic signaling mechanisms and proteins. Future studies would likely involve: (1) testing hit compounds in one or more in vivo animal models; and (2) additional synthesis and testing of structural analogs, and profiling their efficacy and toxicity thresholds in R6/2 (HD) mice.

### Example 3

### Characterization of the Role of Microtubules and Mitochondria in Huntington's Disease

The normal function of huntingtin (htt) and the mechanism of toxicity caused by expanded polyQ stretches are still unclear. Both a gain of novel function and a loss of normal function have been proposed to explain pathology caused by polyQ expansions in htt. Htt has an essential role in embryonic development and neuronal survival. The protein is largely cytoplasmic and is associated to some extent with microtubules (MT) and membranous compartments of the cell. Diverse functions have been proposed for htt because of its interactions with proteins involved in cellular transport (HAP1), cell death (HIPPI), transcription machinery (CBP, TAFII130) and metabolism (GAPDH). Also, cell toxicity shows context dependence since the extreme N-terminal fragments containing the glutamine repeats are more toxic than larger fragments or full length Htt. The mechanism(s) for context dependence are unclear but may be due to altered or novel interactions of different length Htt fragments with protein partners. There is no effective therapy available for HD.

The present Example uses a chemical genetic approach, wherein biologically active small molecules are used to alter gene and protein function and to identify pathways that affect a phenotype of interest. This approach has the additional advantage of identifying drugs and drug targets that may be relevant to disease.

Compounds that cause MT depolymerization and ones that inhibit mitochondrial electron transport rescued cell death in a neuronal cell culture model of mutant-htt-induced neurotoxicity. Previous studies have suggested interactions between huntingtin and both microtubules and mitochondria. The discovery that affecting some of these interactions can rescue cell death provides a connection between cell death, huntingtin and these cellular components. Discovering the basis for these effects will provide a significant advance in this understanding of mutant and wild-type huntingtin-regulated cell death and may lead to identification of targets for therapy that can prevent neuronal cell death in HD.

1. High-Throughput Screening in a Rat Striatal Neuronal Model of HD

As described in Example 1, a high-throughput cell viability assay was developed in a rat striatal neuronal cell model of mutant htt's toxicity. The model uses embryonic rat striatal neurons immortalized by stably transfecting a temperature-sensitive SV40 large T antigen to generate the ST14A cell line. ST14A cells were then engineered to express normal length polyQ (wild type (WT)) or expanded polyQ (mutant) human htt. ST14A cells were engineered to express WT (15Q to 23Q) or mutant polyQ stretches (82Q to 120Q) in N-terminal 63, 548 or 3144 (full length (FL)) amino acids of human htt. (Rigamonti, D. et al. Wild-type huntingtin protects from apoptosis upstream of caspase-3. J Neurosci 20, 3705-3713 (2000)). These different cell lines proliferate comparably at the permissive temperature (33° C.), but upon serum deprivation and a change to a nonpermissive temperature (39° C.), the cells differentiate and undergo cell death over 2-3 days. However, the rate of cell death is dependent on expression of mutant or wild type htt; there is enhancement of cell death in mutant-htt-expressing cells and retardation of cell death in WT-htt-expressing cells.

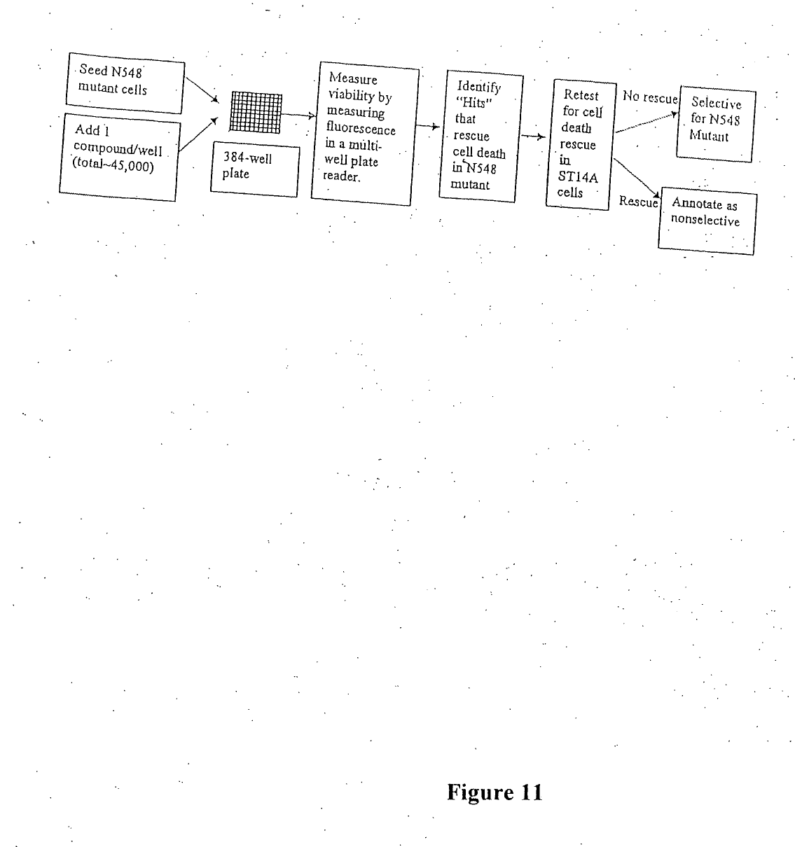

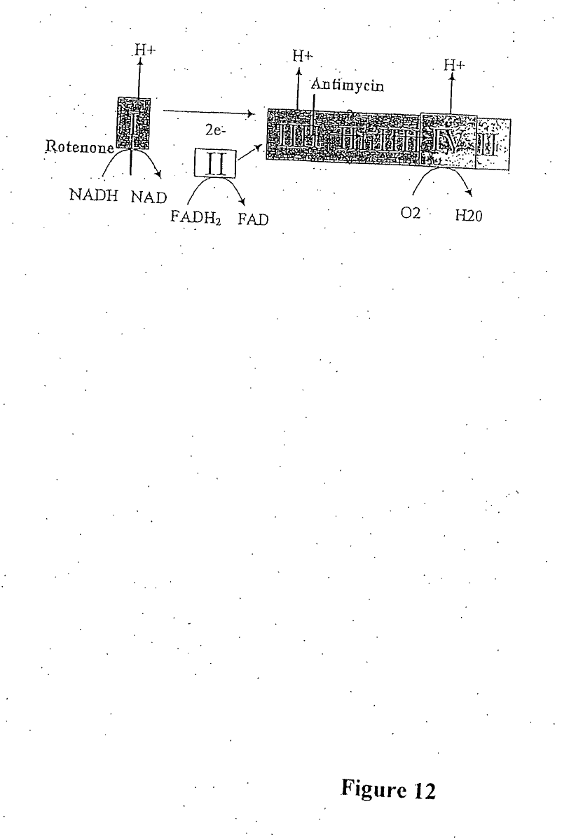

Approximately 45,000 compounds were screened to identify small molecules that selectively prevented cell death in N548 mutant-expressing cells but not in parental ST14A cells (FIG. 11). Compounds identified in the initial screen were retested in a dose series and reconfirmed by testing compounds obtained from a commercial supplier. Further, the compounds discovered were tested for selectivity in cell lines expressing different length htt constructs (with either wild-type or mutant length polyglutamine stretches) to determine context dependence for cell death rescue.

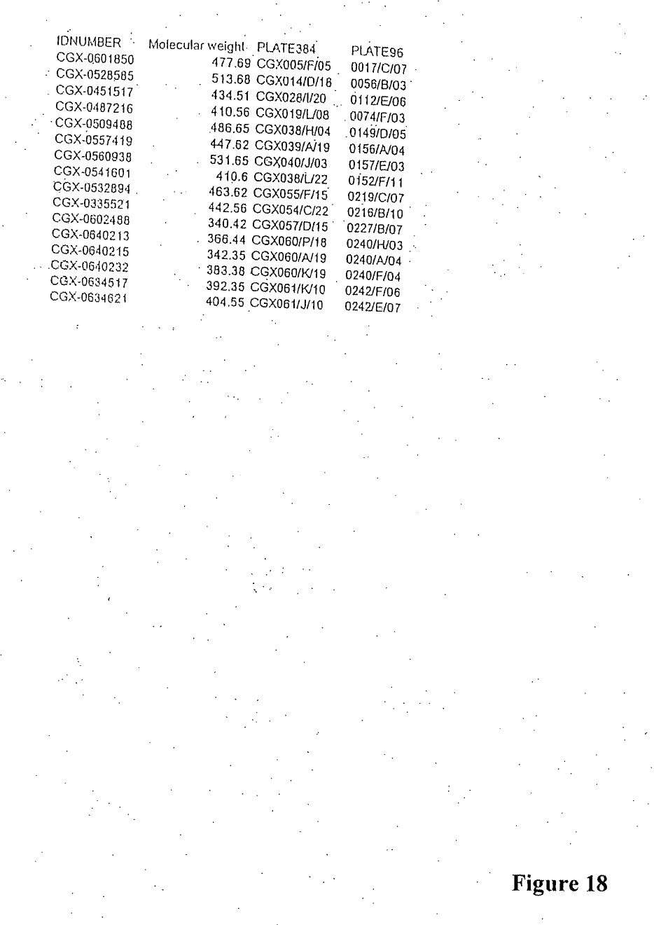

FIG. 18 lists the compounds (analogs) identified using the ST14A cell assay system.

A) Electron Transport Chain (ETC) Inhibitors Prevent Mutant Huntingtin-Induced Cell Death

Specific inhibitors of ETC complex I (Rotenone) and III (Antimycin A) selectively prevented cell death in mutant-Htt (N548 and full length)-expressing cells but not in parent ST14A cells (Table 2 and FIG. 11). Inhibition of complex I or complex III inhibits the net flow of electrons to complex IV and blocks NADH oxidation. Complex I inhibition does not affect electron flow from complex II, whereas complex III inhibition prevents electron flow through both complexes I and II and cause accumulation of NADH and FADH₂.

TABLE 2
Htt Length Dependence for Rescue by Microtubule and
Mitochondrial Inhibitors.

| | mut N63 | mut N548 | mut F1 | WT N63 | WT N548 | WT-F1 |
|---|---|---|---|---|---|---|
| Microtubule inhibitor | − | + | + | − | + | − |
| rescue | | | | | | |
| Mitochondrial | − | + | + | − | − | − |
| inhibitor rescue | | | | | | |

Microtubule destabilizing agents rescue mutant htt-induced cell death

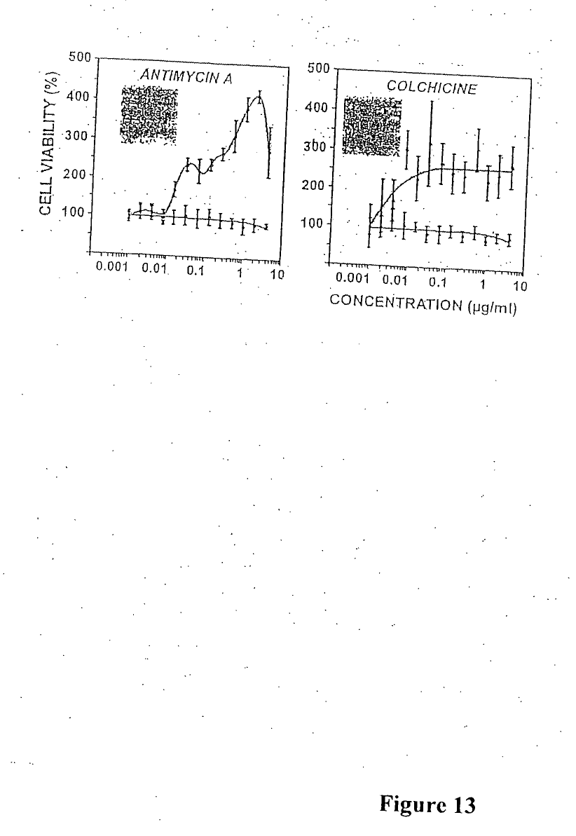

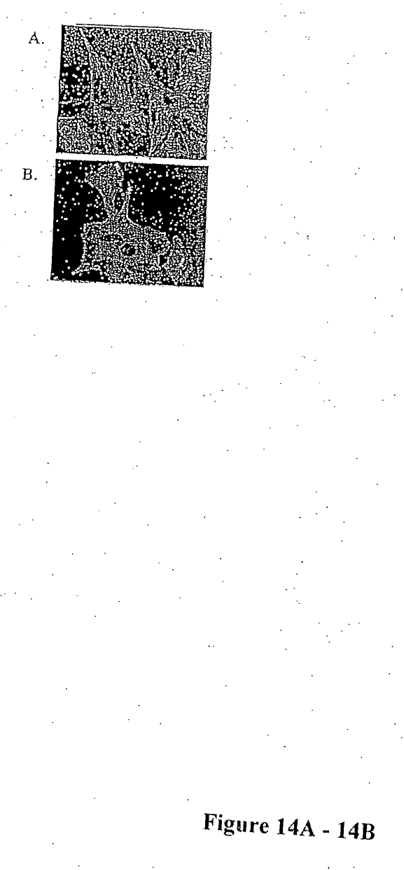

Four structurally diverse MT depolymerizing agents (referred to as MT inhibitors, MTIs) rescued cell death in the mutant-N548-expressing cell line but not in the parent cell line (FIG. 13). These compounds include colchicine, podophyllotoxin, vincristine and nocodazole. Etoposide, a structural analog of podophyllotoxin, with a different mode of action, did not rescue cell death, suggesting MT depolymerization is the relevant mechanism of action for these compounds. MT depolymerization after treatment with these agents was confirmed by indirect immunofluorescence against tubulin (FIG. 14). The profile for cell death rescue by MTIs in the different length versions of mutant and WT is shown in Table 2. Cell death rescue by MTIs was htt length-dependent (Table 2, top row) and was not observed in N63 mutant-expressing cells, suggesting a role for the region between 63 and 548 amino acids in cell-death rescue. This region of htt is important for binding several proteins, suggesting that interactions between htt and other proteins may be involved in the prevention of cell death by MTI. Interestingly, rescue was observed in the N548-wild-type-expressing cells but not the cells expressing full-length wild-type htt, possibly due to polyQ expansion affecting protein interactions in the FL protein, but not of the N548 protein.

2. Defining the Role of Mitochondrial Electron Transport Inhibitors in Alleviating Cell Death in Huntington's Disease

One aim of the studies is to identify the site of mitochondrial/metabolic defect in mutant htt-expressing cells and to characterize the effect of ETC inhibitors on this defect.

Numerous studies have documented mitochondrial defects in HD models. Mitochondria from HD patients reportedly have enhanced sensitivity to complex II and complex IV inhibitors; defects in complex III activity have also been reported. Chemical inhibition of complex II activity causes a HD-like phenotype in rats and primates. However, it is unclear if there are specific or generalized mitochondrial defects in HD and if these defects are secondary to alterations in metabolism.

Mitochondria couple the energy released from oxidation of NADH/FADH₂ into a proton gradient at the electron transport chain (ETC) (FIG. 11) and use the proton gradient to catalyze the synthesis of ATP. Also, mitochondria are at the center of the cell death pathway; the release of key players in cell death including cytochrome c from mitochondria triggers cell death. They are also the principal sites for the generation of toxic reactive oxygen species (ROS) in cells.

The defects that could enhance cell death in mutant htt expressing cells and explain rescue by ETC inhibitors include changes in metabolism affecting NADH/FADH₂levels, defects in the ETC or the generation/protection against ROS.

A) Assaying Metabolic Defects in Mutant Htt Expressing Cell Lines

One hypothesis is that mutant htt alters metabolism by its interaction with GAPDH, a key glycolytic enzyme, leading to decrease in the amount of NADH/FADH₂. A decrease in glycolysis has been implicated in cell death in cell culture models. Also, levels of NADH regulate enzymatic steps that regulate histone acetylation that is implicated in HD. The ETC inhibitors may reverse these defects by causing an accumulation of NADH/FADH₂ and thus be protective. The relative amounts of NADH and FADH₂ will be measured spectrophoto-metrically and compared between N548 mutant and ST14A cells in the presence and absence of the mitochondrial inhibitors. This assay would detect pre-ETC defects in mutant Htt cells.

In case, mutant N548 cells have lower levels of NADH/FADH₂ compared to ST14A cells, the role of this decrease in causing cell death will be tested directly by adding NADH/FADH₂ exogenously to cells and monitoring cell death rescue. In case no differences in NADH/FADH₂ are observed in the two cell lines, it would argue against mutant htt causing a glycolytic defect.

B) Assessing ETC Defects in Mutant-Htt-Expressing Cell Lines

Next, the presence of a primary ETC defect in mutant N548 expressing cells will be determined. The ETC function will be assessed by measuring ATP concentration and MTT reduction in mutant N548 Htt and ST14A cells treated with or without mitochondrial inhibitors. ATP levels reflect the rate of flux of NADH/FADH₂, mitochondrial ETC function and coupling of ETC with oxidative phosphorylation. ATP concentration will be measured using a commercially available Bioluminescence Assay Kit CLSII (Boehringer Mannheim). MTT reduction to MTT formazan as a measure of the reductive potential of the ETC will be assayed by measuring absorbance spectrophotometrically at 570 nm (Slater et al., 1963, Biochim Biophys Acta 77: 383-93). In case both mitochondrial (ETC) and metabolic defects are revealed in mutant expressing cells, the primary site of the defect will be determined by measuring ATP levels, and MTT reduction in isolated mitochondria. These experiments will help determine if the mitochondrial defects are primary or secondary to metabolic defects. In case no ETC defects are detected, it would argue for alterations in cell death regulators caused by mutant htt.

C) Assessing Cell Death Regulatory Activity in Mutant Htt Expressing Cell Lines

Apoptotic signals cause a release of cytochrome c from mitochondria in ST14A cells and ETC inhibitors may rescue cell death by preventing the release of cytochrome c. The inhibition of electron flow to complex IV may be involved in preventing cytochrome c release since cytochrome c is the electron carrier between complex III and IV. The amount of cytochrome c released into the cytosol will be measured in N548-mutant-expressing cells and in parental ST14A cells in the presence and absence of mitochondrial inhibitors by western blotting using a monoclonal antibody against cytochrome c. If ETC inhibitors prevent cyctochrome c release, then it would suggest that mutant-N548-htt-induced release of cytochrome c is dependent on electron transport.

Mitochondria are the major site for production of reactive oxygen species (ROS) that are widely implicated in cell damage and death. Decreased ETC flux by complex I and III inhibitors may be protective by inhibiting the production of excess ROS. ROS production will be assayed in N548 mutant and ST14A cell lines by measuring the conversion of nonfluorescent DCF-DA and DHE dyes to a fluorescent product upon oxidation by ROS. The test compounds will be washed away before adding the dyes and the redundancy of assays will control for test-compound-induced artifacts. If mutant-Htt-expressing cells show increased ROS production that is inhibited by mitochondrial inhibitors, then this result would implicate enhanced ROS production as being causative in HD toxicity. This effect will be confirmed by testing for inhibition of cell death by various ROS inhibitors including N-acetylcysteine, beta carotene, alpha tocopherol and resveratrol in mutant Htt expressing cells. If ROS production is not enhanced but cell death is rescued by N-acetylcysteine in N548-mutant-expressing cells, it would suggest that mutant htt causes defects in the machinery that protect from ROS.

Together, these assays will distinguish between metabolic and primary mitochondrial defects in mutant htt expressing cells. Further experiments will be performed to identify the underlying mechanism(s) by which mutant htt causes those defects.

3. Defining the Role of Microtubule Depolymerization on Mutant Htt's Neurotoxicity

One aim of the studies is to characterize changes in MT and htt-associated proteins upon MT depolymerization.

MTs are a major component of the cytoskeleton and are involved in diverse processes, including cell division, cellular transport and scaffolding of proteins regulating transcription and cell death. Models that could explain the dependence of htt-induced cell death on MT disassembly will be tested. One model is that localization of a cell death regulatory protein to MTs is altered via interaction with mutant htt. A second model is that mutant htt's interaction with a protein involved in cell death is regulated by MT dynamics. In either model, MT disassembly would change the interactions between a cell death regulatory protein and htt or MTs and result in the inability of mutant htt to induce cell death. A number of predictions of these models can be tested. First, this death regulatory protein should bind differentially to MTs in the mutant N548 compared to the parent cell line. Second, there should be a change in the association of this protein with htt upon MT disassembly. Third, the N63 htt construct is predicted not to interact with this protein(s) and/or associate with MTs. Finally, the interactions of this death regulatory protein should be similar in mutant and WT N548 but should be different in the corresponding versions of full-length htt.

A) Identification of MT/Htt-Associated Cell Death Regulators

Initially, differences between proteins associated with MTs in mutant N548 and parent cell line will be characterized. Tubulin will be immunoprecipitated (IP) from mutant N548 expressing and ST14A cells using a beta-tubulin antibody. In other experiments, the exogenous N548 mutant protein will be immunoprecipitated using antibodies that recognize an expanded polyQ epitope in htt or an N-terminal human htt-specific antibody with and without MTI treatment of cells. The epitope specificity of the antibodies will ensure that the endogenous rat wild type htt protein is not immunoprecipitated. Also comparison of the proteins immunoprecipitated using two antibodies raised against different epitopes of htt will reduce false positives. Relevant controls will include immunoprecipitated ST14A cell lysates with htt specific antibodies and immunoprecipitation of mutant N548 cells with nonspecific control antibody. The immunoprecipitated proteins will be separated by SDS PAGE, analyzed by silver staining and differentially precipitated proteins will be identified by protein microsequencing.

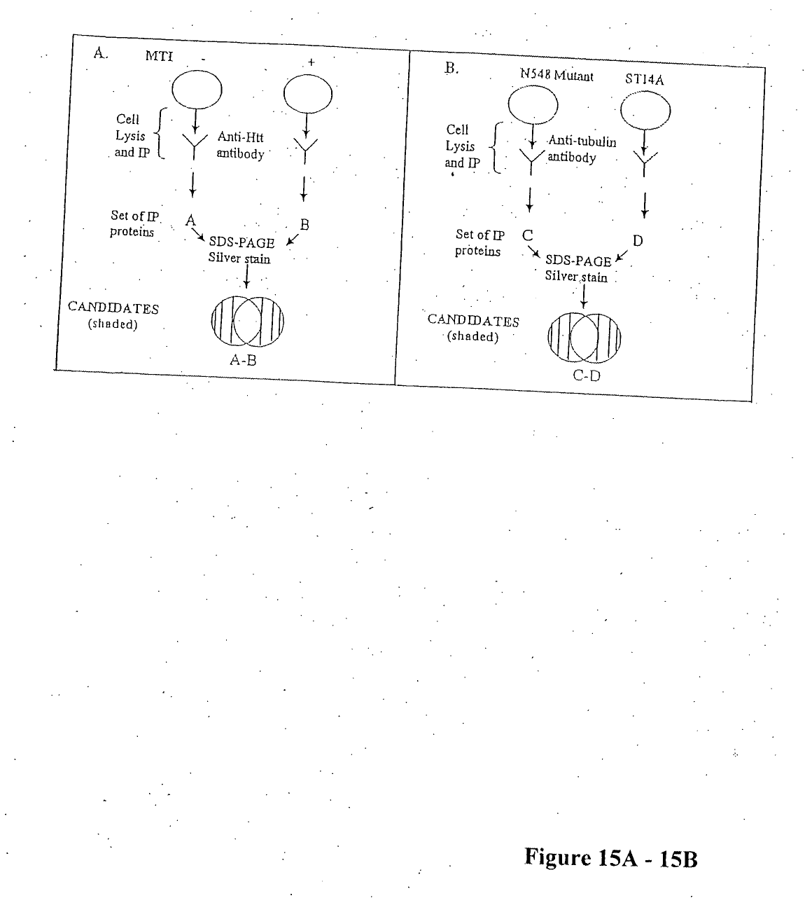

In addition, protein levels will be assessed in the IP by Western blotting for known tubulin and htt interactors, including htt, htt interacting proteins HAPI, HIPI, and cell death regulators that interact with MT, BIM1 and survivin. Any proteins found to be differentially associated with MT in the two cell lines (mutant N548 and ST14A) or showing altered binding with htt upon MTI treatment would be potential candidates for a role in cell death rescue (FIG. 15). Next, the identified candidates' association with MT/htt in the different length htt expressing cell lines will be characterized to test if a candidate protein's association profile matches the rescue phenotype seen in that cell line. For example, N63-expressing cells should show association similar to the parent cell line since this cell line is not rescued by the MTI. This approach will reduce the number of potential candidates to relevant ones. The role of these candidates in cell death rescue will be confirmed by RNAi based loss of function and cDNA overexpression studies in the presence and absence of MTI.

The potential problems with immunoprecipitation assays are optimizing the amount of cell lysates, duration and time of incubation of antibody with the lysates. This would be addressed by testing two well-established antibodies that have been used for htt immunoprecipitation and titrating different amount of cell extracts at different temperature (4° C., 15° C. and 25° C.) that will be incubated with the antibody for different times (from 1 hour to 24 hours).

Another aim of the studies is to test the effects of mutant Htt on MT-based transport and the effect of disruption of transport on mutant-Htt-induced toxicity.

B) Measuring the effect of mutant Htt on mitochondrial localization and transport MTs serve as a scaffold for vesicular, organelle and protein transport. MT-based transport is accomplished by plus and minus end directed motor protein complexes that transport cargo to or away from the cell periphery, respectively. Htt has been proposed to play a role in vesicular/protein transport in part due to its association with HAP-1, a protein that interacts with dynein, a minus end directed motor protein complex. Altered MT-based transport has recently been implicated in HD pathology. MT disruption could rescue cell death due to disruption of htt-dependent transport of a cell death regulator.

Mitochondria, key players in cell death, change from diffuse to perinuclear localization on receiving apoptotic signals. Mitochondrial localization and transport are regulated by MT-based motor activity and will be assayed to detect alterations in mutant-htt-expressing cells. Mitochondrial localization will be assayed in live cells by staining mitochondria with the vital fluorescent dye MITO tracker (Molecular Probes) and transport recorded by time lapse fluorescence video microscopy. First, the localization and rate of transport of mitochondria will be compared between the parent and different length htt expressing cell lines to determine htt length or polyQ dependence on these metrics. Second, the effect of MT depolymerization on these parameters will be assayed in the different cell lines. Third, the effect of inducing cell death at 39° C. in the different cell lines on mitochondrial localization will be monitored. In case defects in the mitochondrial transport are detected in mutant htt cells, experiments will be directed to detect if the transport defects are due to alterations in plus or minus ended motors components and if these changes cause cell death.

C) Testing the Effect of Disrupting Dynein and Kinesin Based Motor Transport on Mutant Htt-Induced Cell Death

To directly test the effect of disrupting MT-based transport on cell death rescue, the minus and plus end directed MT-based motor complexes will be inhibited and their effect on cell death assayed. Overexpression of dynamitin, a component of the dynein motor complex, disrupts the activity of this complex by a dominant negative effect, whereas expression of dominant negative Kinesin Light Chain (KLC) disrupts kinesin, a plus end motor protein complex. MT transport will be disrupted by transiently expressing these constructs in the mutant N548 and ST14A cell line and the rescue of cell death will be assayed by double immunofluorescence for the expressed protein and DNA stain Hoechst 33258 (Sigma) to visualize DNA condensation and fragmentation. Mitochondrial transport in the transfected cells will be assayed to control for inhibition of transport by these constructs. In case the results show cell death rescue by blocking transport, experiments to decrease the levels of specific dynein/kinesin based transport proteins using a RNAi based knockdown of dynactin and kinesin motor protein using lentiviral expression vectors will be initiated. These will be made available as this laboratory is part of a consortium at Whitehead Institute/MIT that is making a mammalian RNAi library. Decrease in the levels of the targeted proteins will be monitored by western blotting. In case no defects in MT-based transport are observed and the disruption of MT transport does not rescue cell death, these results would argue against a role for mutant htt in altering MT-based transport and would favor models of mutant htt's altered association with a death regulator.

In summary, mitochondrial inhibitors and microtubule depolymerizers as specific inhibitors of mutant htt induced neurotoxicity were identified. Experiments are designed to address the localization of mitochondrial defects in mutant htt expressing cells and the mechanism of rescue by ETC inhibitors. In other experiments, the identity of proteins that change association with htt and MTs upon MT depolymerization will be ascertained. Also, experiments will be performed to address the effect of disrupting MT-based transport on the mutant htt's neurotoxicity and determine alterations in MT transport due to mutant htt. The information from the experiments above will enhance this understanding of HD pathology and provide drug targets that can prevent HD toxicity.

### Example 4

### Selective Small Molecule Inhibitors of Cell Death in Mutant-Huntingtin-Expressing Neuronal Cells

Huntington's disease (HD) is one of at least nine inherited neurological disorders caused by trinucleotide (CAG) repeat expansion (others being Kennedy's disease, dentatorubro-pallidoluysian atrophy, and six forms of spinocerebellar ataxia). One aim of these experiments is to identify small molecule suppressors of PolyQ neurotoxicity and to elucidate mechanisms of polyQ neurotoxicity through studying the functional means by which the identified compounds suppress polyQ-expanded Htt toxicity.

As described in Example 1, expressing polyQ-expanded huntingtin protein (Htt) in cultured rat striatal neuronal cells (ST14A cells ) led to selective toxicity over wild-type Htt expressing cells. Using this ST14A model assay system, approximately 47,000 compounds were screened for their ability to prevent polyQ-mediated toxicity.

4.1 Experimental Procedure

Cell Culture. The striatal neuronal cell lines were maintained as previously described (Rigamonti, D., et al., 2000, J Neurosci 20, 3705-13). PC12 cells expressing mutant htt (exon 1 with 103Q) under an ecdysone inducible promoter were a gift from Erik Schweitzer (Aiken, C., et al., 2004, Neurobiol Dis 16, 546-55). They were passaged in PC12 media (DMEM with 10% horse serum and 10% fetal bovine serum) at 37° C. in 9.5% CO₂. Mutant htt was induced by the ecdysone receptor agonist, tebufenozide (Aiken, C., et al., 2004, Neurobiol Dis 16, 546-55).

Compound Libraries. Approximately 47,000 compounds were screened. These included FDA-approved drugs and known biologically active compounds from NINDS (1,040 compounds, Microsource Discovery Inc.) and ACL (2,036 compounds) (Root, D. E., et al., 2003 Chem Biol 10, 881-92) collections, 20,000 synthetic compounds from a combinatorial library (Comgenex International, Inc) and 23,685 natural, semi-natural and drug-like compounds of unknown biological activity from diverse sources (Timtec, Interbioscreen and Chembridge). All compounds were prepared as 4 mg/ml solutions in DMSO (dimethylsulfoxide) except NINDS compounds (10 mM), in 384-well plates (Grenier, Part no.781280). “Daughter plates” were prepared from stock plates by a 1:50 dilution in serum free DMEM (3 μl compound to 147 μl DMEM) in 384-well plates (Grenier, Part no.781270).

Screening and data analysis. 1500 cells were seeded in 384-well plates (Costar 3712) in 57 μl of media with 0.5% inactivated fetal calf serum (IFS). 3 μl of each compound was transferred from daughter plates to triplicate assay plates for a final assay concentration of 4 jig/ml (10 μM for NINDS compounds) in 0.1% DMSO. All transfers were conducted using a robotic Advanced Liquid Handler (Sciclone, Zymark). Cells were then incubated at 39° C. After 3 days, cells were washed 10 times with phosphate buffered saline (PBS) leaving 20 μl residual PBS per well, and 20 μl of 2 μg/ml calcein AM (Molecular probes) in PBS was added per well. Cells were incubated at room temperature for 4 h and fluorescence (ex 485/em 535 nm) intensity was measured using a plate reader (Packard). The fluorescence intensity in each well was normalized to the median of each plate. The median normalized fluorescence of each triplicate assay well was determined. A 50% increase in intensity above the median signal intensity was considered a “hit”. For the PC12 assay, 7500 cells/well were plated in 384-well plates in 57 μl of PC12 medium with tebufenozide (1 μM). Compounds (3 μl) from daughter plates were added to the cells and incubated at 37° C. After 48 h, 20 μl of 40% alamar blue (Biosource, CA) in media was added/well and cells incubated for 12 h at 37° C. Cell viability was assayed by measuring Alamar blue reduction (ex 530/em 590 nm) in a plate reader (Perkin Elmer Victor³)

Cell viability assay (trypan blue exclusion): N548 mutant or ST14A cells were plated at 10⁶ cells in 10 cm tissue culture plates, media changed to serum deprived DMEM (0.5% IFS) and cells incubated at 39° C. for 48 h after treatment with DMSO or compounds. Cells were trypsinized and subjected to an automated trypan blue (0.4%) exclusion cell viability assay (Vi-Cell 1.01, Beckman Coulter). At least 1,000 cells were counted in each assay and the percentage of trypan blue negative (viable) cells was calculated for each assay. For PC-12 cells, 10⁶ cells were plated and viability was determined using the trypan blue exclusion assay after 42 h of htt-Q103 induction.

Western blot analysis and antibodies. Cell lysates were prepared and subjected to western blotting as previously described (Dolma et al. 2003). Anti-T antigen (Santa Cruz, Pab 108), β-tubulin (Sigma, clone TUB2.1), anti-huntingtin (Chemicon Intl. MAB2166 and 1C2), anti-cleaved caspase-3 (Asp175), anti-cleaved caspase-7(Asp198) (Cell Signaling Technology), goat anti-mouse HRP and goat anti-rabbit HRP (Santa Cruz) antibodies were used.

Indirect Immunofluorescence. Cells were grown on glass coverslips in 10% IFS-containing media, treated with compounds and fixed in acetone/methanol (1:1 vol/vol). β-tubulin was detected using a mouse antibody (clone TUB2.1, Sigma) followed by a rhodamine-conjugated goat anti-mouse antibody (Jackson laboratories). Cells were viewed under a fluorescent microscope (ex 530/em 595 nm).

Indirect Immunofluorescence. Cells were grown on glass coverslips in 10% IFS-containing media, treated with compounds and fixed in acetone/methanol (1:1 vol/vol). β-tubulin was detected using a mouse antibody (clone TUB2.1, Sigma) followed by a rhodamine-conjugated goat anti-mouse antibody (Jackson laboratories). Cells were viewed under a fluorescent microscope (ex 530/em 595 nm).

Fluorogenic caspase assay. Caspase activity was measured using a fluorogenic assay (Biovision Inc. CA), based on cleavage of AFC (7-amino-4-trifluoromethyl coumarin) from specific AFC-conjugated peptide substrates by activated caspases. Each cell line was seeded at 10⁶ cells/plate, incubated overnight at 33° C., and then incubated for 6 h at 39° C. in 0.5% IFS containing medium with or without 50 μM BOC-D-fmk (Biomol). Four plates/sample were harvested in lysis buffer provided by the manufacturer. Peptide substrates were added to the cell lysate or to lysis buffer (control), incubated at 37° C. for 2 h, and fluorescence (ex 355/em 510 nm) measured on a plate reader (Perkin Elmer Victor³). Fluorescence intensities of controls were subtracted from sample intensity and the resulting values normalized to protein in each sample (Bradford assay-Biorad).

C. elegans neuronal survival assay. 100 synchronized L1 animals (pqe-1;Htn-Q150 ) (Wood 1988; Faber et al. 2002) were added to 5 wells (20 animals/well) of a 96 well plate. Each well contained 50 μl food suspension (6.6 O.D.) pre-mixed with compound or DMSO in a 96-well plate. C. elegans were incubated for 2 d at 15° C., washed in S-media, immobilized with 5 mM sodium azide on a microscopic glass slide and GFP fluorescence was examined using an Axoplan2 fluorescence microscope (ex 485/em 535 nm). Live (GFP positive) Anterior Sensory Horn (ASH) neurons were counted in at least 50 animals (100 neurons). Data were subjected to a two-tailed Student's t-test.

Rat Brain Slice HD assay. Degeneration of medium spiny neurons (MSNs) in brain slice explants was induced by biolistic transfection of htt constructs based on previously published approaches. (Khoshnan, A. et al. Activation of the IkappaB kinase complex and nuclear factor-kappaB contributes to mutant huntingtin neurotoxicity. The Journal of neuroscience: the official journal of the Society for Neuroscience. 24, 7999-8008 (2004)). At postnatal day 10, brains were dissected from CD Sprague Dawley rats (Charles River) after euthanasia and sliced into 250 micron coronal sections containing striatum using a tissue microtome (Vibratome). All animal experiments were done in accordance with the Institutional Animal Care and Use Committee and Duke University Medical Center Animal Guidelines. Brain slices were plated onto serum-supplemented culture medium and maintained at 32° C. degrees under 5% CO2 as previously described (Khoshnan 2004); compounds were added to the culture medium at the time of plating. DNA constructs (encoding Yellow Fluorescent Protein (YFP), Cyan Fluorescent Protein (CFP), and htt-Q73-CFP containing the full exon 1 domain of human htt, a 73 polyglutamine repeat, and a CFP fusion at the C-terminal) were purified and coated onto 1.6 micron elemental gold particles and delivered to the brain slice explants using a biolistic device (Helios Gene Gun, Bio-Rad) as previously described. MSNs co-transfected with YFP+htt-Q73-CFP degenerate over the course of 4-7 days compared to control neurons transfected with YFP+CFP only. On day 5 after explantation and transfection, MSNs were identified based on their position within the striatum and on their characteristic morphology using fluorescent stereomicroscopes (Leica). Those MSNs that expressed bright, even YFP fluorescence and showed 2 or more dendrites with continuous YFP labeling at least 2 cell body diameters in length were scored as healthy.

Photomicrographs: N548 mutant or ST14A cells were plated at 10⁶ cells in 10 cm tissue culture plates and media changed to serum deprived DMEM (0.5% IFS) and incubated at 39° C. for 48 hours with DMSO or compound treatment. Cells were viewed under a phase contrast microscope and images were acquired with a CCD video camera (Optronics Engineering, Goleta. Calif.).

Equations. Calcein fluorescence of cells on the day of seeding to represent 100% rescue was used. The difference in calcein fluorescence signal of cells on the day of seeding and after increasing number of days under SDM at 39° C. was determined in 3 independent runs of the assay and used to calculate Z′. (I) CV (coefficient of variation)=100*(SD/Mean); (II) Z′ factor=1-(3*SD₁+3*SD₂)/(Mean₁-Mean₂).;1=control, 2=positive outcome. SD=standard deviation.

4.2 Experimental Results

Assay development and Characterization of Striatal HD model. An assay for detecting cell death in mutant-htt-expressing striatal neurons in a 384-well-plate format was developed. Calcein-AM-based methods were used to detect cell viability, and optimized cell number and time course for the assay. Calcein AM is a cell permeable non-fluorescent dye that is cleaved by cellular esterases to generate fluorescent calcein, and is retained by live cells (Wang, X. M., et al., 1993, Human Immunology 37, 264-70). This assay has the advantage of a wash step that removes compounds before calcein addition and thus limits false positives from fluorescent molecules.

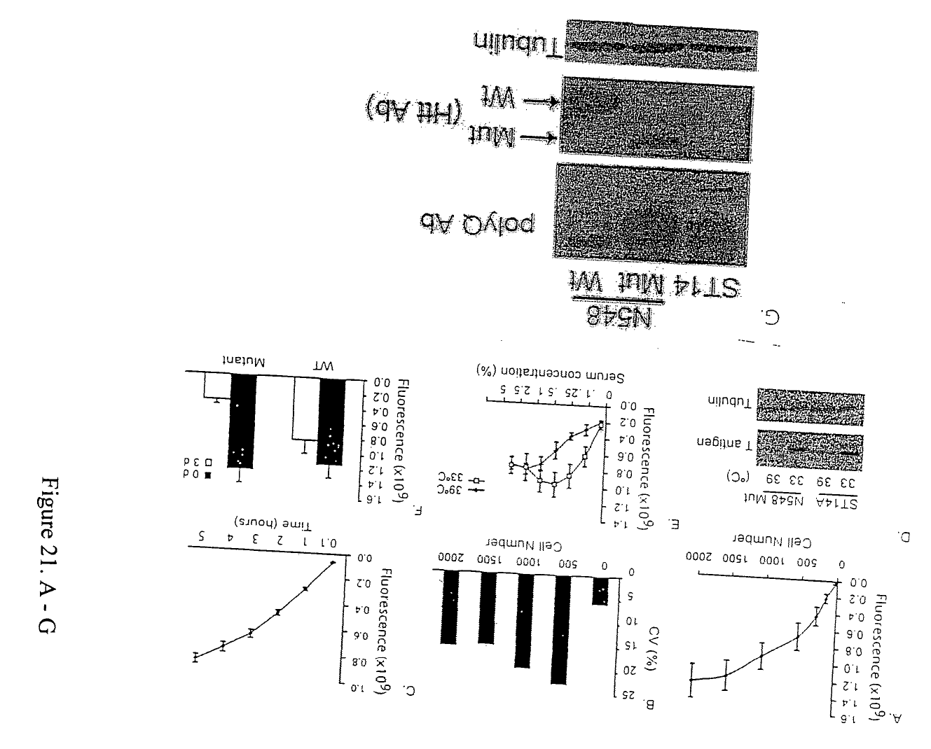

A cell number titration was performed to determine the cell density at which calcein fluorescence was not saturated and the coefficient of variation (CV) was low (FIGS. 21A and 21B). A low CV is critical for a high-throughput assay as it decreases noise and enhances sensitivity. Based on this analysis, 1500 cells per well were chosen for the assay. Next, the kinetics of the fluorescence signal. Calcein signal increased linearly over 4 h (FIG. 21C) was determined, giving a time window for performing the assay in a high-throughput manner. Thus, with a read time of 4 minutes for a 384-well plate, about 60 plates could be processed serially within 4 h of calcein AM addition.

Four key features of the ST14A model were confirmed: (1) expression of the N548 mutant and N548 WT htt transgene, 2) temperature-dependent degradation of T-ag, 3) cell death upon serum deprivation, and 4) the protective effect of WT htt. It was confirmed N548 mutant and WT htt expression by western blotting using two antibodies; one antibody detects expanded-polyQ-containing htt (1C2) and a second antibody detects both WT and mutant htt (MAB2166). The three cell lines (ST14A, N548 mutant and N548 WT) were analyzed for the expression of transgene by western blotting. The antibody specific for expanded polyQ detected a band at the expected molecular weight (between 100-120 kd) in the N548 mutant cell line, but not in ST14A or N548 WT cell lines (FIG. 1C, top panel). The htt antibody (MAB2166) detected the truncated htt protein in both N548 mutant and N548 WT cells, but not in ST14A cells (FIG. 21 G). N548 mutant protein had a decreased mobility compared to the N548 WT, likely due to the extra polyQ length in the mutant protein. Also, the three cell lines had similar expression of the endogenous rat htt These results confirmed transgene expression in N548 mutant and N548 WT. Western blotting for T-ag (FIG. 21D) showed that a shift to 39° C. decreased T-ag protein within 6 h in both parental and mutant cell lines, consistent with degradation of T-ag at 39° C.

Next, the serum concentrations required for inducing cell death in these cells were determined. N548 mutant cells were seeded in 384-well plates at a density of 1,500 cells per well, in decreasing concentrations (5 to 0%), of inactivated fetal-calf serum (IFS), and incubated at 33° C. or 39° C. Cell viability was assayed after 3 d using the calcein AM assay (FIG. 21E). N548 mutant cells at 33° C. and 39° C. had similar viability in serum concentrations above 1.0% suggesting that higher temperature alone did not affect cell viability. Serum concentrations between 0.25% and 1.0% selectively decreased cell viability in differentiated (39° C.) mutant cells, while lower serum concentration affected viability of undifferentiated cells (33° C.) as well. Therefore, 0.5% serum for inducing cell death was chosen in the screen. Finally, it was confirmed that in 0.5% IFS, N548 WT cells were protected relative to N548 mutant cells (FIG. 21F). Additionally, serum deprivation (0.5% IFS) decreased viability in the parental ST14A cells to levels below WT and slightly above N548-mutant-htt cell lines after 2-3 days. These results were consistent with previously published results (Rigamonti, D., et al., 2000, J Neurosci 20(10): 3705-3713) and validated the adaptation of the assay to a 384-well plate format.

Assay optimization for high-throughput screening (HTS). Next, it was determined if the assay adapted for the 384-well format was suitable for high-throughput screening. The Z′ factor is a measure of the quality of a high-throughput assay (Zhang J., et al., 1999, Journal Of Biomolecular Screening 4: 67-83.); calculation of Z′ is provided in Materials and Methods. A Z′ value greater than 0 is required for a usable assay, with a maximum value of 1.0 for an ideal assay. Calcein fluorescence of cells on the day of seeding to represent 100% rescue were used. The difference in calcein fluorescence signal on the day of seeding and after increasing number of days under serum deprivation was determined and used to calculate Z′. After 3 d of serum deprivation, the assay had a Z′ between 0.1 and 0.25, the lower range for an assay suitable for HTS. Since multiple replicates decrease the false negative rate (missed hits), without increasing the false positive rate (Zhang J. H., et al., 2005, J Biomol Screen 10(7): 695-704.), it was decided to perform screening in triplicate to enhance the reliability of data obtained.

Though all screening was performed in the above format, recent improvements on the reliability of the assay (Z′ consistently above 0.35, FIG. 31) were made by a modification of the assay. This modified assay measures cellular dehydrogenase function to assess cell viability 25 and dispenses with washing steps, a significant source of variability in the Calcein AM based viability assay. This improved format would be useful for screens using this assay in the future.

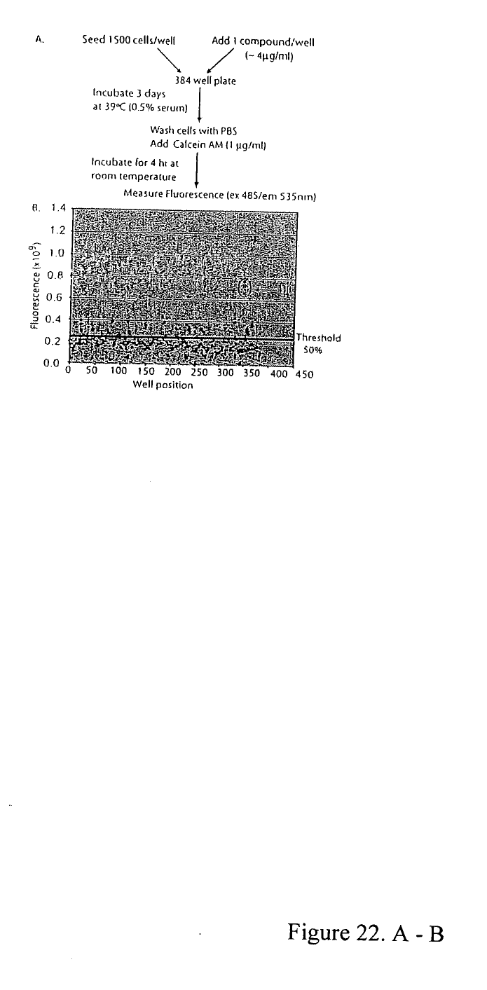

Pilot screening: Before performing large-scale screening, the robustness of the assay was tested by screening the NINDS library (1,040 compounds). The NINDS library was screened in triplicate in two independent runs at a final concentration of 10 μM. Tthe criterion >50% increase in fluorescence above median plate fluorescence in at least two of three replicates were defined to identify a “hit”. A flow diagram of the assay and data from one run of NINDS screening is shown in FIG. 22A and 22B. Data from each run were analyzed independently. A total of 80 positives were identified, with 58 and 56 identified from the first and second independent runs, respectively. Of these 80 positives, 34 were identified in both runs, while 46 were unique in the two runs. All 80 positives were subsequently tested in triplicate in dose-dilution experiments (16-point, 2-fold dilution series). All hits confirmed by this dose-response assay were re-ordered from the vendor and re-tested in two independent dose-dilutions assays, each performed in quadruplicate. 32 hits were reconfirmed by these criteria giving a confirmation rate of 40% (32/80). Of these 32 hits, 21 were in the mechanistic class of glucocorticoids. There were 26 glucocorticoids in the NINDS library (a complete list of compounds in the library is available at the website http://ninds.nih.gov/funding/areas/neurodegeneration/NINDS_Drug_Screening.htm), of which 21 were identified in the screen, suggesting a low false-negative rate for this mechanistic class. Since on average, glucocorticoids caused a 100% increase in viability, this false-negative rate is likely higher for compounds with weaker activity. Of the 32 reconfirmed hits, 26 were common to both runs of the screening assay, suggesting that ˜80% of hits that were eventually confirmed could be identified in a single run of the assay performed in triplicate. This analysis suggested that the assay was reproducible in multiple runs, had a reasonable hit confirmation rate (˜40%) and a low false negative rate for relatively strong hits. Further, a single run, performed in triplicate, identified the majority of hits likely to be detected. It was concluded that the assay was sufficiently robust to undertake a large-scale screen.

Another 46,000 compounds were assayed in the same 384-well plate format in triplicate; hits were identified by the criteria described above. All hits were confirmed by re-testing in dose-response (16-point, 2-fold dilution series) experiments in triplicate. These compounds were reordered from the vendor and then assayed for activity in repeat dose-response assays performed in quadruplicate in at least 3 independent experiments. Based on these criteria, 50 compounds were identified that prevented cell death in N548 mutant htt cells.

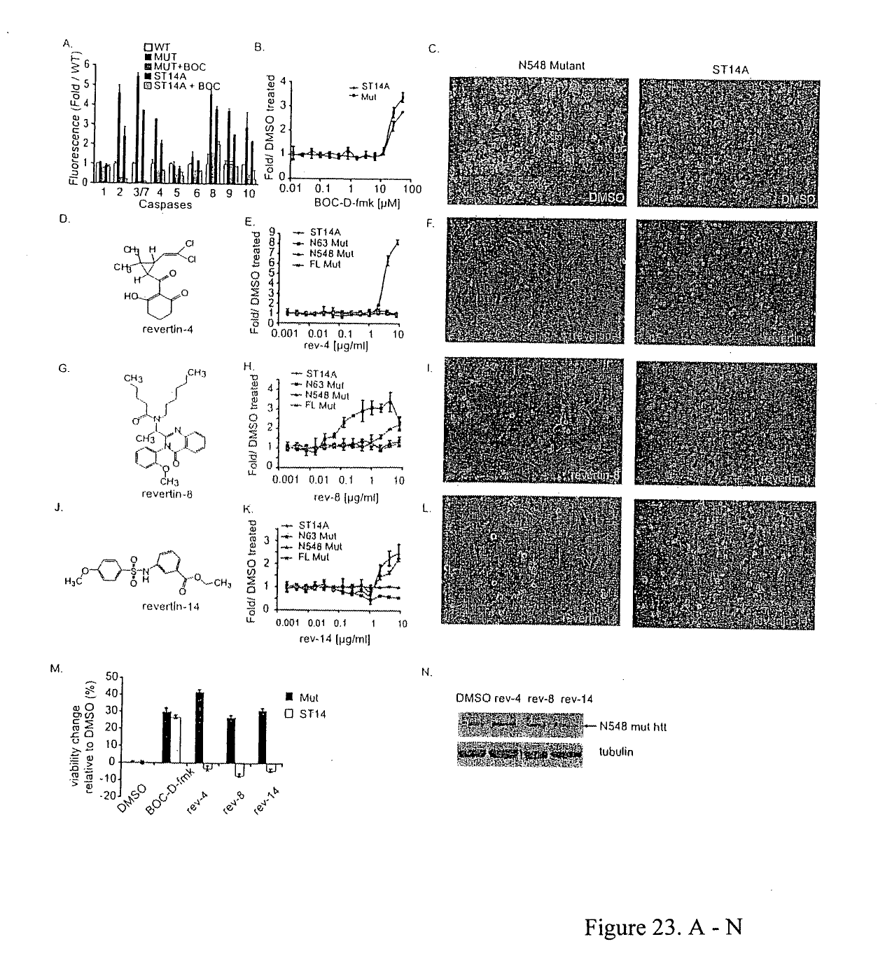

Secondary Viability assays. To confirm that enhanced cell viability determined by calcein fluorescence was a true reflection of an increase in cell viability, secondary viability assays were performed for all hits using Trypan blue exclusion and by monitoring changes in cell morphology. This Trypan blue cell viability assay is based on dye-exclusion by live cells, but not cells whose membrane integrity is compromised. All hit compounds were also tested for their ability to reverse morphological aspects of cell death: under conditions of serum deprivation at 39° C., a substantial number of cells round up and detach between 24 and 48 h, and are easily seen using light microscopy (FIG. 23C). The ability of compounds to prevent rounding up and detachment was noted. Based on these analyses, 46 compounds were confirmed as enhancing cell viability by all three of the following viability criteria: enhanced calcein fluorescence signal, trypan blue exclusion and cell morphological rescue.

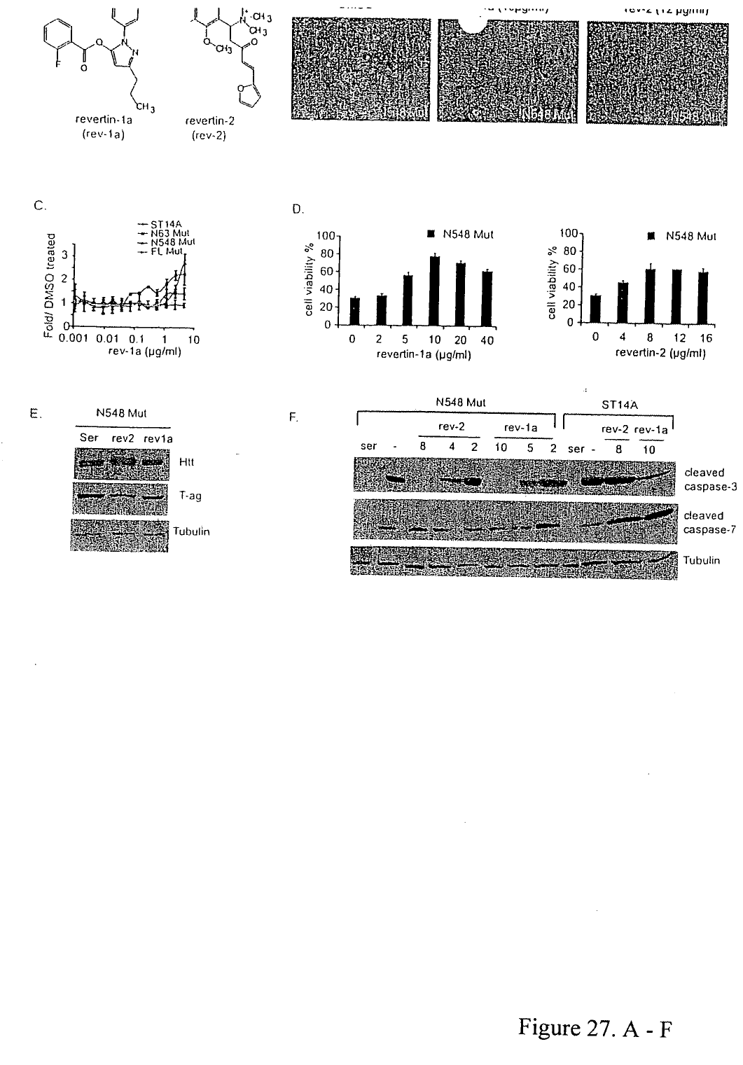

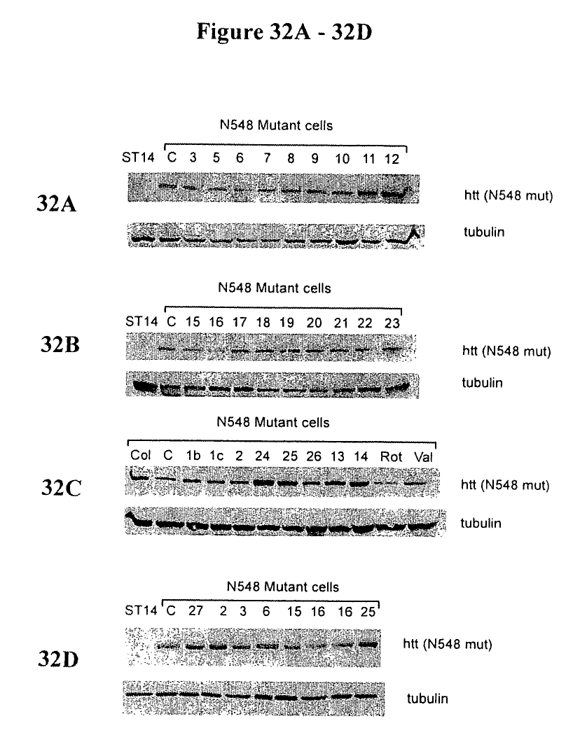

Identification of selective inhibitors of mutant-htt toxicity. As a first step towards probing the mechanisms of action of these compounds, whether the compounds suppressed a general death mechanism or selectively targeted pathways that are perturbed by mutant htt was tested. Caspase-dependent pathways have been implicated in cell death in general and specifically in HD (Thornberry, N. A., and Lazebnik, Y., 1998, Science 281(5381): 1312-1316; Sanchez Mejia, R. O., and Friedlander, R. M., 2001, Neuroscientist 7(6): 480-489 ; Hickey, M. A., and Chesselet, M. F., 2003, Neuropsychopharmacol Biol Psychiatry 27(2): 255-265). Whether caspase activation contributed to cell death in this model was tested. Caspase activation was assayed by measuring cleavage of specific fluorogenic caspase substrates, as well as by western blotting for the cleaved active fragments of caspase-3 and caspase-7, effectors of caspase-dependent pathways. Consistent with previous reports (Rigamonti, D., et al., 2000, J Neurosci 20(10): 3705-3713), enhanced caspase activation in N548 mutant compared to ST14A cells, and inhibition in N548 WT-htt cells (FIG. 23A) was observed. A similar set of specific caspases were activated in both N548 mutant and ST14A cells, as shown by the fluorogenic caspase assay and cleavage of both caspase-3 and caspase-7 by western blotting (FIG. 27F). N548 WT cells have less caspase activation; this correlated with relative protection of these cells from cell death. Furthermore, BOC-D-fmk, a pan-caspase inhibitor (Deas, O., et al., 1998, Journal of Immunology 161(7): 3375-3383.), prevented caspase activation and rescued cell death in both ST14A and N548 mutant cells (FIG. 23A and 23B). None of the selective compounds decreased mutant htt protein expression by western blotting (FIG. 32A-D). These selective compounds likely modulate cell viability pathways that are specifically perturbed by mutant htt.

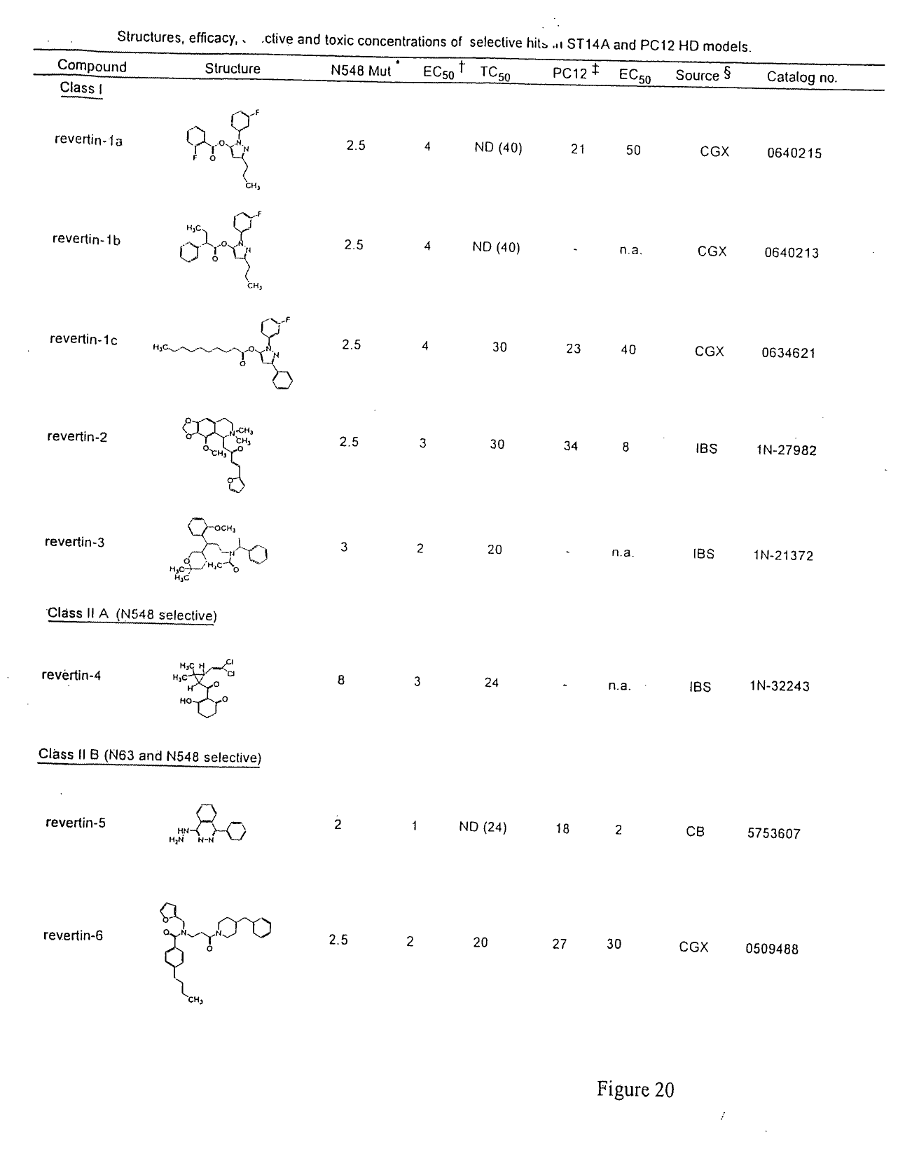

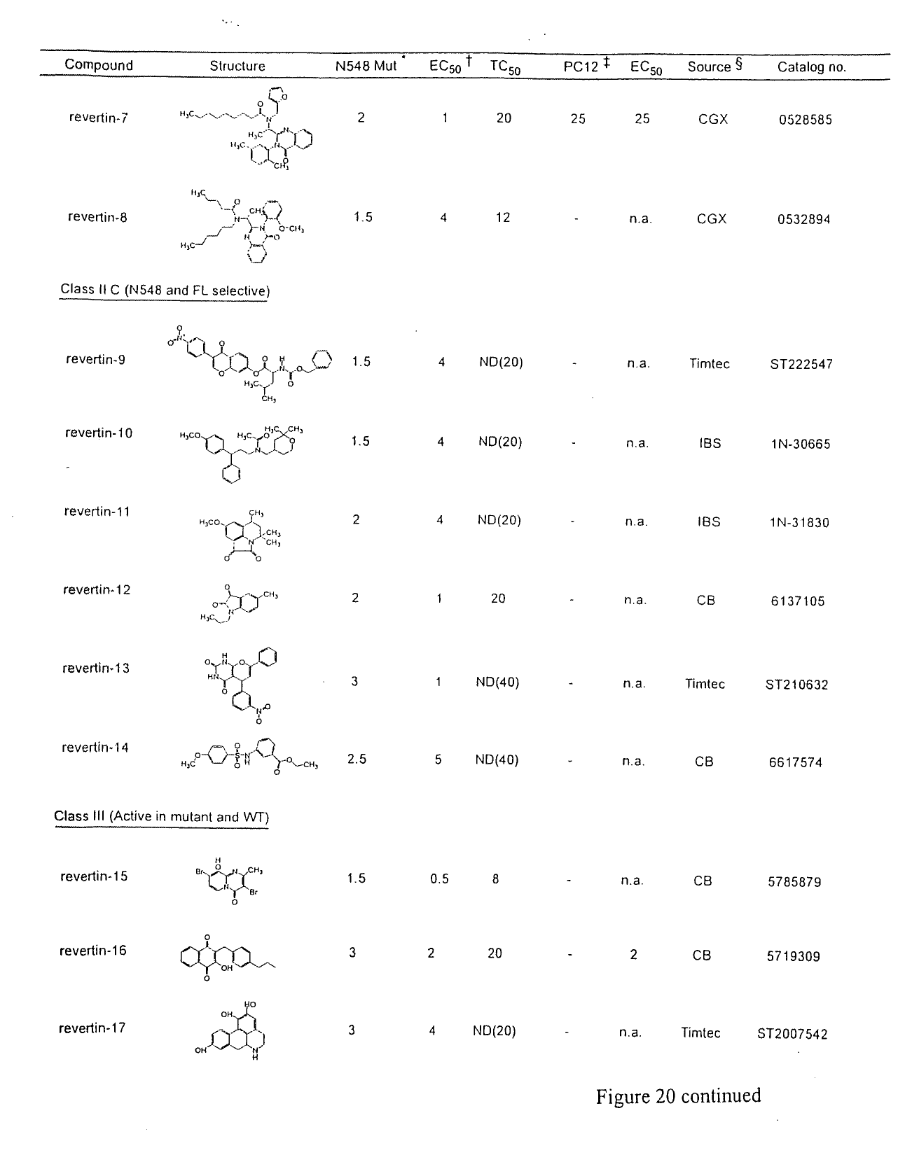

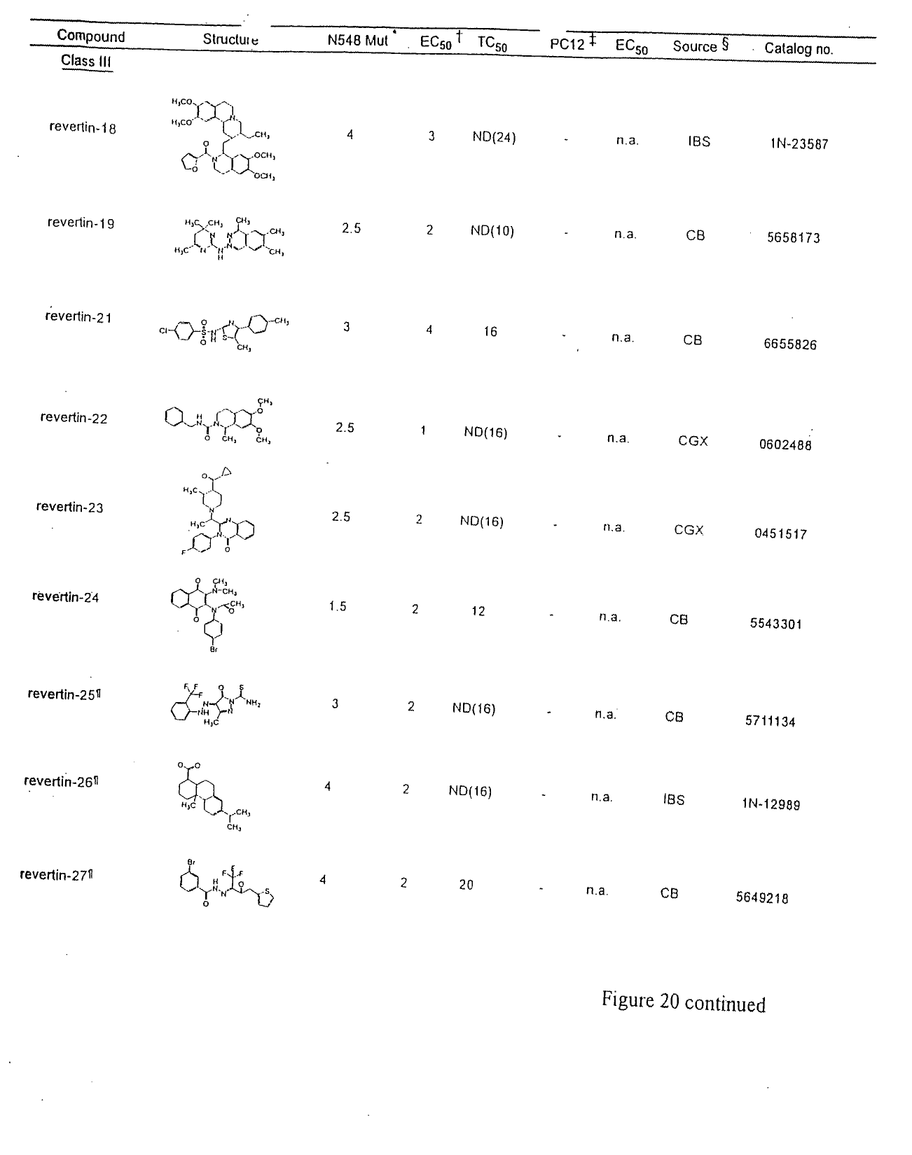

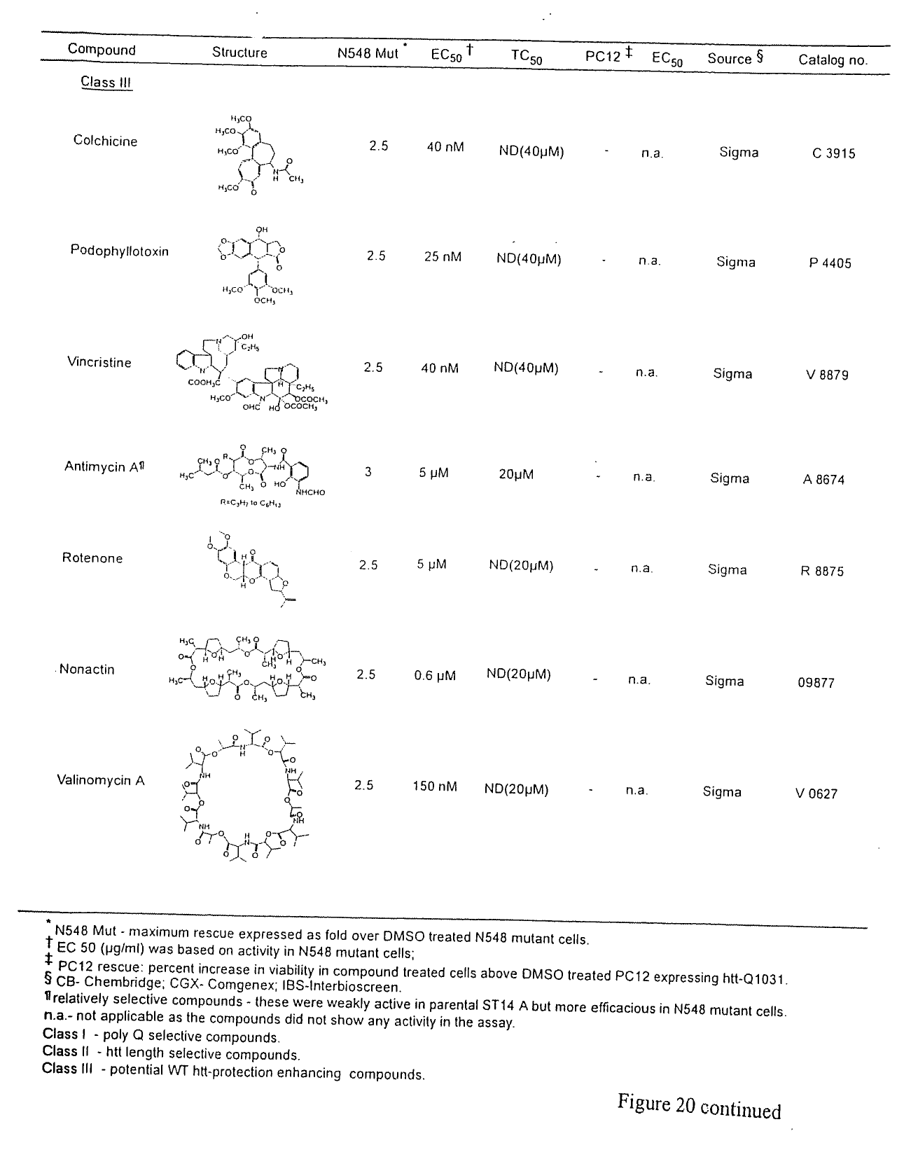

It is concluded that serum-deprivation-induced caspase activation contributes to cell death in this model, but is not specific for cell death in mutant htt-expressing cells. However, the mechanisms leading to caspase activation may differ in the N548 mutant compared to the ST14A cells. Thus, it was decided to use the rescue of cell death in ST14A cells as a selectivity “filter” to identify compounds that were selective for mutant htt-induced cell death pathways. Ten compounds with known biological mechanisms were identified as non-selective protective agents (FIG. 24) in both cell lines. Some of these are FDA-approved drugs (budesonide, clofibrate and tretinoin) while others have a history of clinical use (flufenamic acid and zaprinast) (Kagan, G., et al., 1981, Journal of International Medical Research 9(4): 253-256; Reiser, J., et al., 1986, British Journal of Diseases of the Chest 80(2): 157-163) or are currently used for treating rare metabolic diseases with neurological symptoms (tetrahydrobiopterin) (Bernegger, C., and Blau, N., 2002, Molecular Genetics & Metabolism 77(4): 304-313). Thus, these drugs may have potential for neurodegeneration therapy, despite lacking selectivity for cell death in mutant-htt-expressing cells. A few compounds, such as prostaglandin E2 and the chloride channel blocker 2-NPPB, are neuroprotective in other neuronal cell culture models (Wei, L., et al., 2004, European Journal of Physiology 448(3): 325-334; Gendron, T. F., et al., 2005, European Journal of Pharmacology 517(1-2): 17-27), validating, to some extent, this assay for discovering general neuroprotective agents. However, 36 compounds were also identified that selectively suppressed cell death in mutant-htt-expressing cells (for compound structures, vendor information, efficacy and effective concentrations see supplemental FIG. 20). Of these compounds, 29 were novel; these compounds were named “revertins” for reversal of mutant huntingtin toxicity (revertin1a,b,c through revertin 19, and revertin 21 through revertin 27). Revertins may modulate pathways affecting cell viability that are perturbed by mutant htt (FIG. 20).

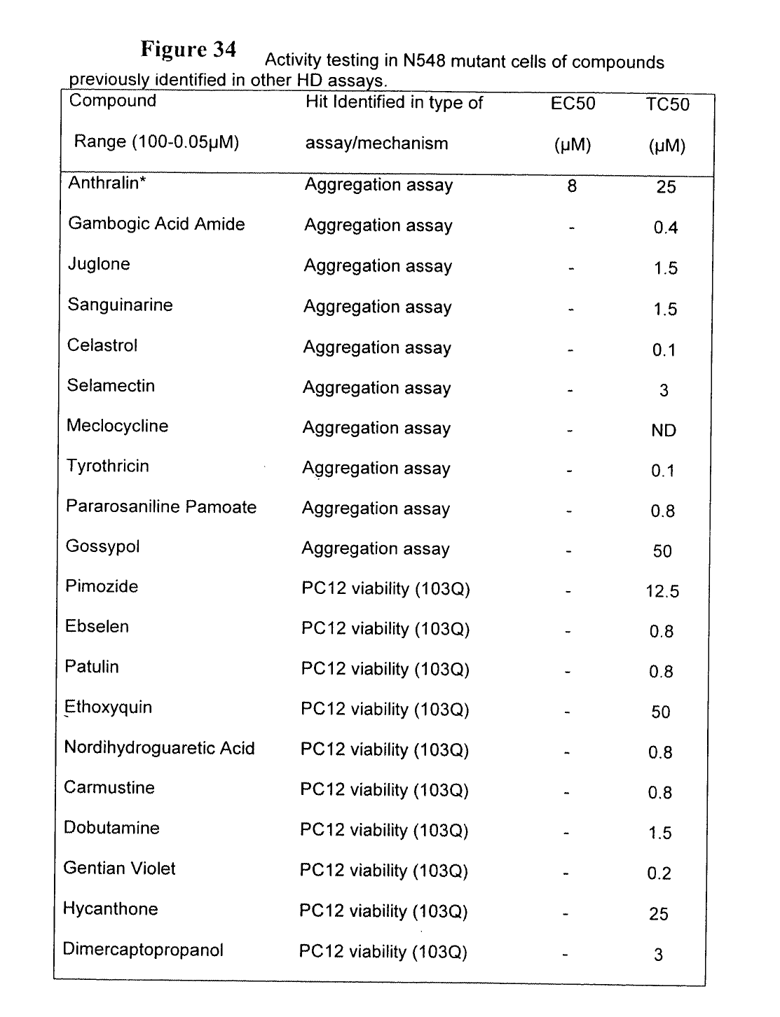

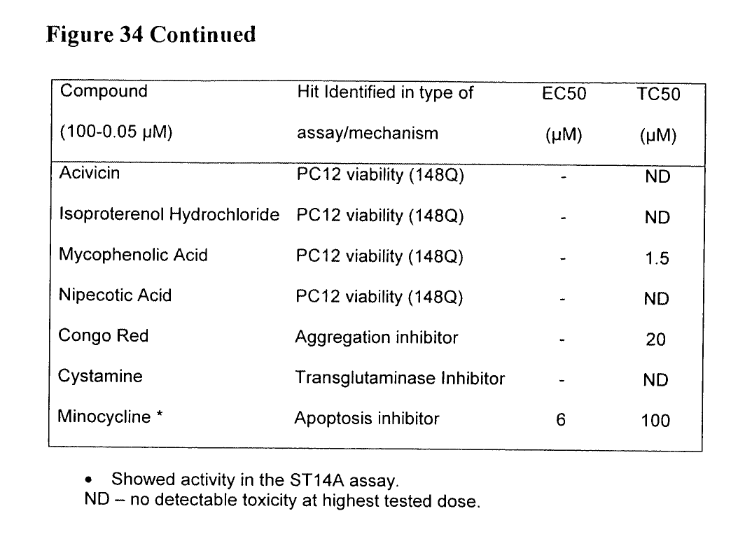

The NINDS compound collection has been previously screened and a number of hits identified in different HD assays (PC12 viability and aggregation assays) (Aiken, C. T., Tobin, A. J. & Schweitzer, E. S. A cell-based screen for drugs to treat Huntington's disease. Neurobiol Dis 16, 546-555 (2004); Wang, W. et al. Compounds blocking mutant huntingtin toxicity identified using a Huntington's disease neuronal cell model. Neurobiol Dis 20, 500-508 (2005); Wang, J., Gines, S., MacDonald, M. E. & Gusella, J. F. Reversal of a full-length mutant huntingtin neuronal cell phenotype by chemical inhibitors of polyglutamine-mediated aggregation. BMC Neurosci 6, 1 (2005); Apostol, B. L. et al. A cell-based assay for aggregation inhibitors as therapeutics of polyglutamine-repeat disease and validation in Drosophila. Proceedings of the National Academy of Sciences of the United States of America. 100, 5950-5955 (2003)). Whether any of these compounds were active and showed selectivity in the ST14A model were tested. However, none of the previously described hits in the screen (except BOC-D-fmk) were identified, possible due to the screen being conducted at a single concentration (10 μM). Therefore, 27 commercially available compounds that were identified in other HD screens in N548-mutant cells were tested, over a wide concentration range (100 to 0.05 μM) in a 2-fold dose-dilution series in triplicate (AIken 2004; Wang 2005; Apostol 2003). Hits were confirmed by trypan blue exclusion assay (FIG. 34). Only 2 compounds, anthralin and minocycline were active in N548-mutant cells. Anthralin is a DNA intercalator that is used topically in skin disorders while minocycline has previously shown to be active in the ST14A model by an antiapoptotic mechanism. (Wang, X. et al. Minocycline inhibits caspase-independent and -dependent mitochondrial cell death pathways in models of Huntington's disease. Proc Natl Acad Sci USA 100, 10483-10487 (2003); Wang 2005). However, both compounds failed to show selectivity for N548-mutant cell death. This suggests that the mechanisms targeted by compounds in these different HD assays are distinct.

Toxicity Testing. To assess the therapeutic window for these compounds, compound toxicity in N548-mutant cells was assessed. A dose-response assay was performed with a 13-point, 2-fold dose-dilution; the highest concentration tested being the solubility limit of a compound or a maximum dose of 80 μM (compounds with known mechanism) or 80 μg/ml (novel compounds) for soluble compounds. The approximate TC50 was estimated from dose response curves and this information for all compounds is provided in FIG. 24 (non-selective compounds) and FIG. 20 (selective compounds).

Identification of htt-length-dependent inhibitors of mutant htt toxicity. To define further the mechanisms of action for these compounds, the compounds were tested for activity in striatal cell lines expressing mutant or wild type polyQ in the context of different htt protein lengths. This strategy had three major objectives. First, previous work suggested that htt protein length (context) affects polyQ toxicity (Chan et al. 2002; Yu et al. 2003). Understanding the mechanisms underlying context dependence is important, since smaller htt fragments, in addition to full-length (FL) protein, are reported in HD patients and HD mouse models (DiFi\dot{g}lia et al. 1997; Wellington et al. 2002; Zhou et al. 2003). Second, an observation that two compounds rescue cell death in a different subset of cell lines, would suggest that these compounds act by different mechanisms. Third, since these cell lines constitutively express mutant htt, it is possible that the selectivity is due to a artifactual cell-line-specific change in cell death pathways and not due to a mutant-htt-perturbed pathway. Thus, activity in multiple mutant-htt-expressing cell lines makes it more likely that a mutant-htt-specific cellular pathway is targeted by selective hits.

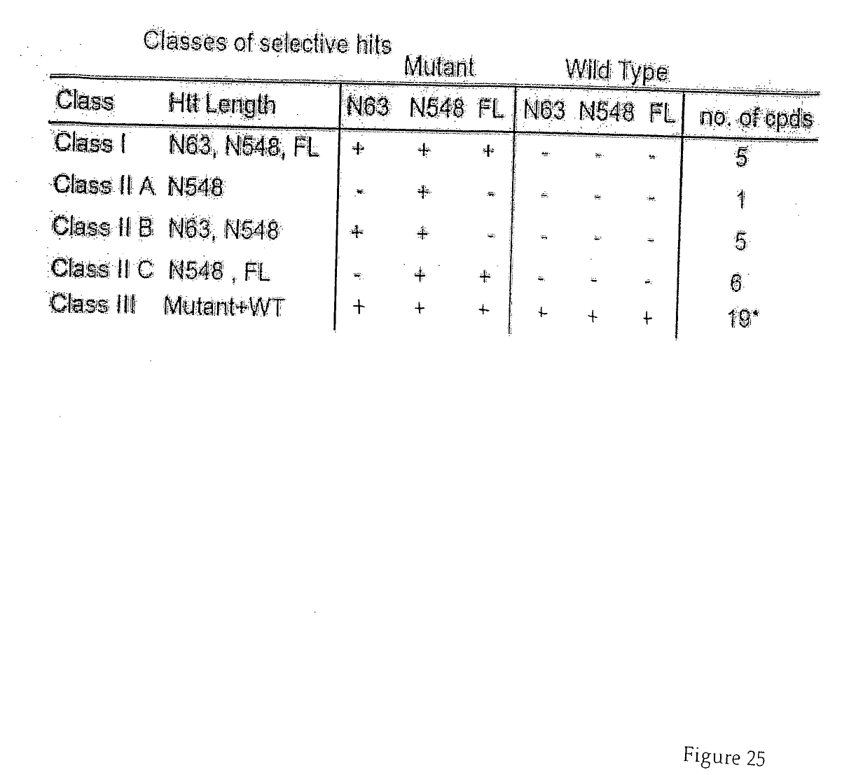

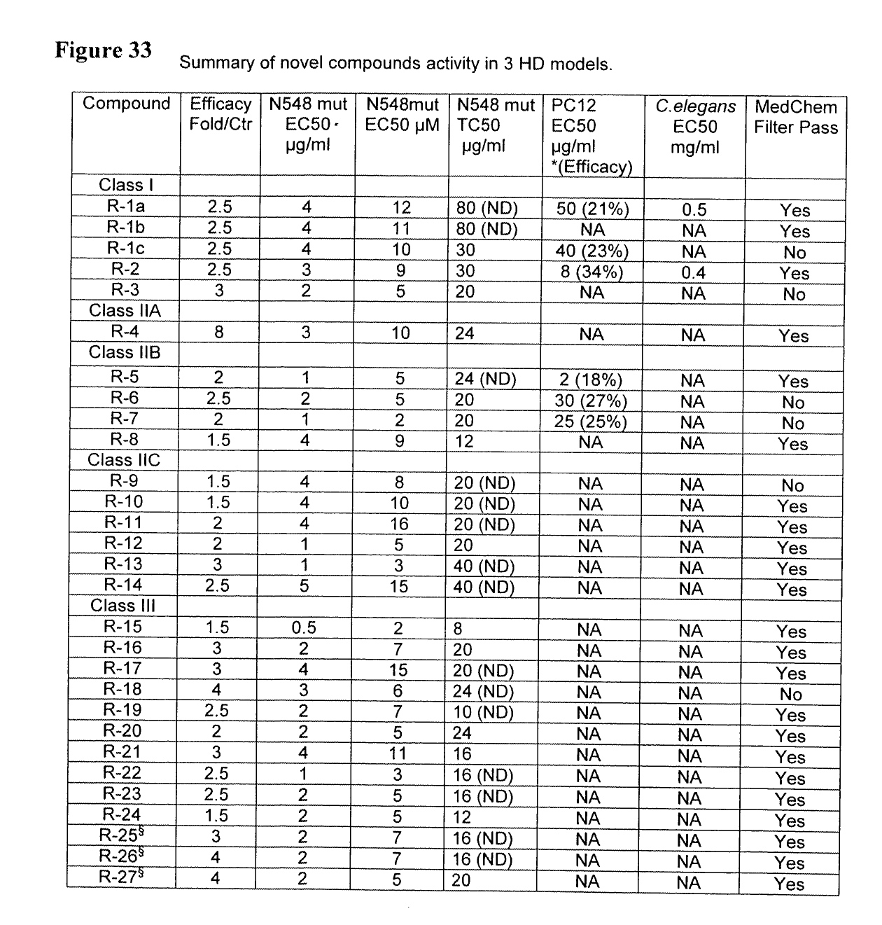

The ability of compounds to rescue cell death in cell lines that expressed expanded (mutant) or unexpanded (WT) polyQ stretches in the context of N63, N548 or FL htt (Rigamonti et al. 2000) were tested. The panel of cell lines in which a compound prevented cell death was referred to as a “selectivity profile”. Based on the results of this testing, revertins were grouped into 3 classes of selectivity profiles (FIG. 25). Compounds that suppressed death in the context of all mutant htt proteins, irrespective of htt length (N63, N548 and FL) were designated class I (5 compounds). Compounds that suppressed cell death in an htt-length-dependent manner were designated class II (11 compounds); these compounds were subdivided into 3 subclasses, based on their efficacy in cells expressing different lengths of mutant htt, namely, compounds that rescued cell death in N548-mutant-expressing cells only (class IIA: 1 compound), compounds that rescued cell death in N63 and N548 mutants (class IIB: 4 compounds) and compounds that rescued cell death in N548 and FL mutants, but not N63 mutant (class IIC: 6 compounds). Dose response curves of the rescue for representative compounds for each class (class IIA, IIB and IIC), along with their structures and micrographs showing morphological rescue are shown in FIGS. 23D to 23L. The selectivity of rescue, that is, rescue in N548-mutant cells, and not in ST14A cells, was confirmed by Trypan blue exclusion assay (FIG. 23M). In addition, these compounds did not affect the expression of mutant htt (FIG. 23N), ruling this out as an explanation for their selectivity. Finally, compounds that suppressed cell death in both mutant htt and WT htt-expressing cells (at least one cell line expressing mutant or WT htt in any context) were designated as class III. A few class III compounds demonstrated relative selectivity, since they alleviated death in ST14A cells but to a lesser extent than in N548 mutant cells (FIG. 33). Based on this analysis, compounds were identified that utilize at least 5 distinct mechanisms to rescue mutant htt toxicity.

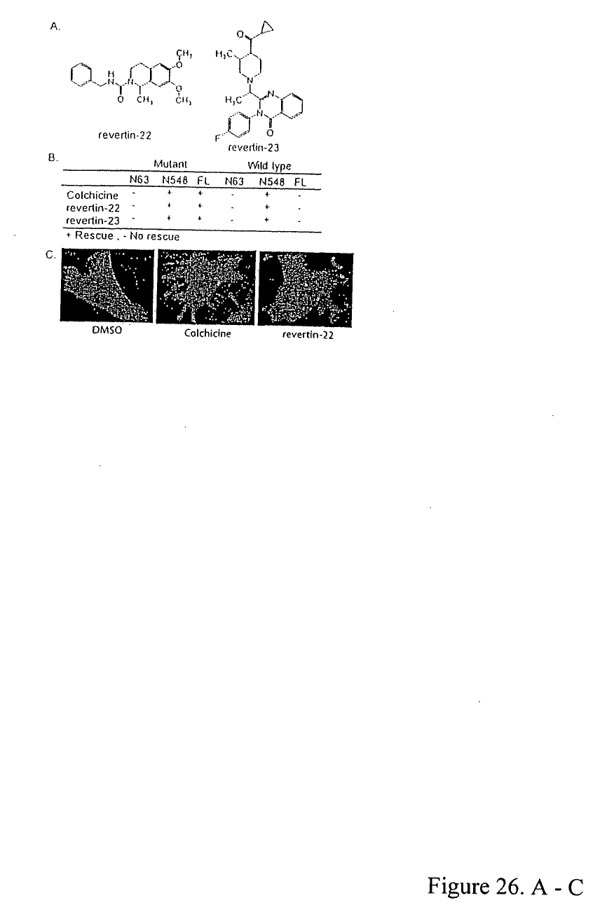

Identification of novel microtubule inhibitors based on selectivity profiling. If the classification of compounds based on differential rescue in an htt-length-dependent manner has underlying mechanistic basis, then compounds in the same class are likely to act by a similar mechanism. In the screening, it was discovered that microtubule inhibitors (MTIs), which depolymerize microtubules, such as colchicine (Jordan, A., et al., 1998, Med Res Rev 18, 259-96), specifically rescue cell death in three cell lines: N548 mutant, FL-mutant and N548 WT (FIG. 26B). Two novel compounds, revertin-22 and revertin-23 (FIG. 26A), shared this selectivity profile with MTIs, but were structurally distinct from known MTIs (Jordan, A., et al., 1998, Med Res Rev 18, 259-96; and FIG. 20). It was hypothesized that these novel compounds may act as MTIs, based on their selectivity profile. Using β-tubulin immunofluorescence, it was discovered that these novel compounds disrupt microtubules (data shown for revertin-22, FIG. 26C). Furthermore, the dose response of cell death rescue by these compounds paralleled that for MT disruption, suggesting that MT disruption is the mechanism by which these compounds rescue cell death. Investigations of this mechanism of selective death rescue by MTI's is ongoing. These findings indicate that selectivity profiling can be used to identify the mechanism of action of novel compounds and suggests an underlying mechanistic basis exists for this classification.

Secondary screening and drug lead discovery. In order for a compound to be an attractive drug lead for further testing in mouse models of HD, it is valuable to observe efficacy in more than one HD model. Such compounds are likely to affect conserved mechanism of htt toxicity. The revertins were tested for rescue of HD phenotypes in diverse models; a neuronal cell culture model (PC12), a yeast HD model, and a in vivo C. elegans (worm) HD model.

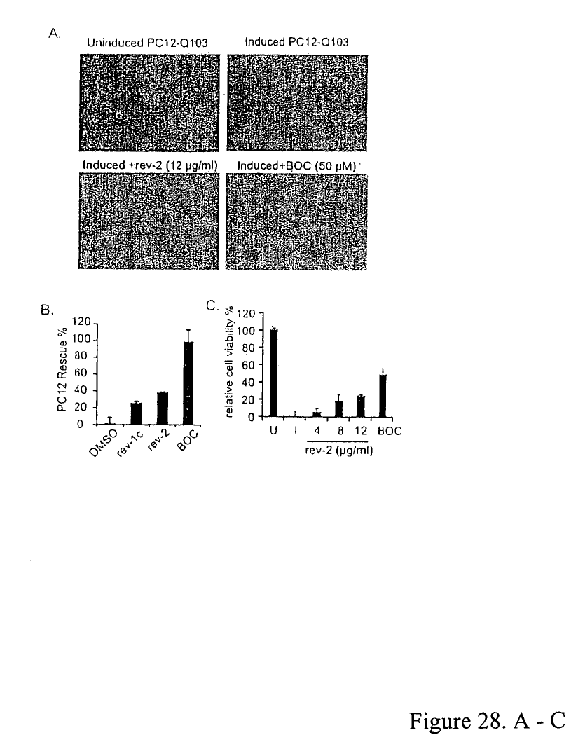

PC12 HD model. All compounds were tested in an HD model in PC12, a cell line of neuroendocrine origin that is extensively studied. In this model, inducible expression of mutant htt (exon 1 with Q103) with an ecdysone receptor agonist, tebufenozide, causes cell death over 48-72 h in PC12 cells (Aiken, C. T., et al., 2004, Neurobiol Dis 16(3): 546-555). The decrease in cell viability was confirmed by morphological criteria, assay of mitochondrial function (alamar blue reduction) and trypan blue dye exclusion assay. PC-12 Htt-Q103 cells round up, detach and start to lyse after 36-48 h of Htt-Q103 induction (FIG. 28A). This results in decreased mitochondrial function (alamar blue reduction) (FIG. 28B) and increased cell membrane permeability (increased Trypan blue staining). The alamar blue assay was used to test viability owing to its amenability to HTS in 384-well format (Nociari, M. M., et al., 1998, Journal of Immunological Methods 213(2): 157-167). Dose-response experiments were performed for all hits in triplicate in two independent experiments. Consistent with an earlier report (Aiken, C. T., et al., 2004, Neurobiol Dis 16(3): 546-555), BOC-D-fmk rescued cell death in this model as evaluated by different viability parameters (FIG. 28C). Since this assay showed less than 5% CV, any compound showing ˜15% increase (+3SD) in alamar blue reduction in both experiments was considered a hit. 7 compounds showed mild to moderate activity in this model, with the most active compounds showing ˜35% rescue in this assay (FIG. 20). Interestingly, all 7 compounds that were active in PC12 model were active in the striatal HD model in an htt-length-independent manner or were selective for N63-mutant and N548-mutant htt cells, again suggesting that the htt-length-based classification had an underlying mechanistic basis.

Yeast HD Model. Inducible expression of an exon1htt transgene with 72 glutamines (Q72) reduces yeast (S. cerevisiae) growth compared to growth of uninduced or Q25-expressing yeast cells (Meriin, A. B., et al., 2002, J Cell Biol 157(6): 997-1004). Compounds were tested for their ability to rescue the growth defect of Q72-htt yeast. Q72-htt yeast cells were engineered in genetic backgrounds with deletions in multi-drug resistance genes (PDR) to enhance drug influx (Bauer, B. E., et al., 1999, Biochimica et Biophysica Acta 1461(2): 217-236). All compounds were tested in a two-fold dilution series, starting at the highest soluble concentrations. None of the compounds rescued yeast growth reproducibly. This indicates that mechanisms targeted by these compounds are likely not conserved in this simple eukaryotic model HD system.

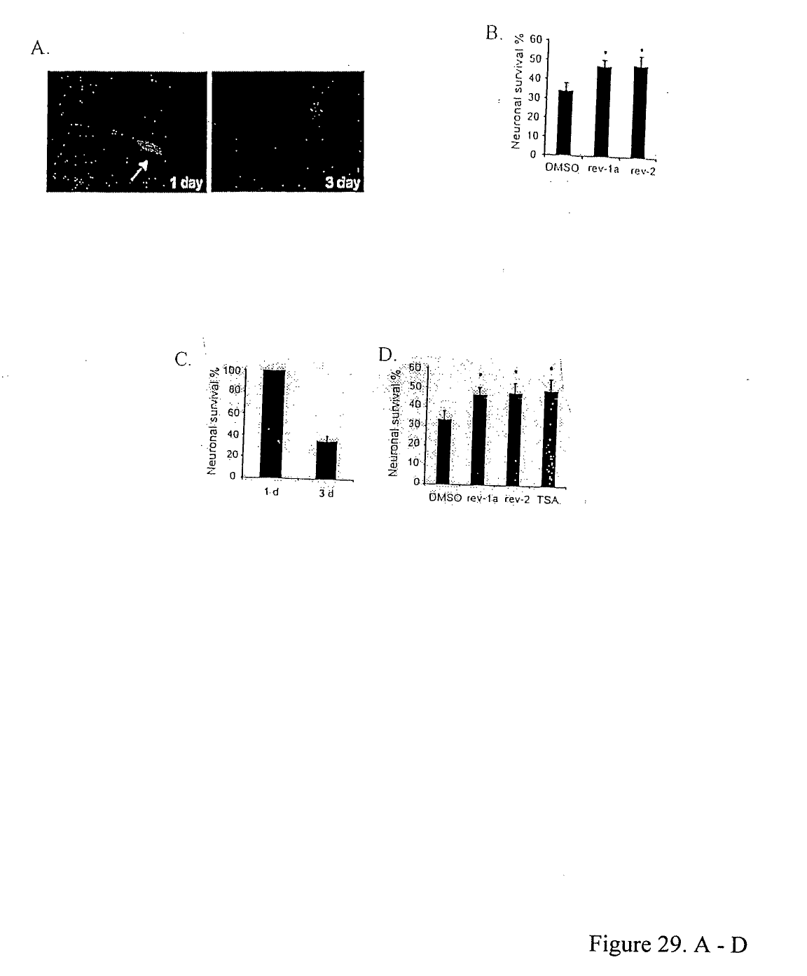

C.elegans (worm) HD Model. A C.elegans HD model was optimized for drug testing. In this model, animals in a polyQ enhancer-1(pqe-1) background that express mutant htt in larval anterior sensory horn (ASH) neurons, undergo ASH neuronal death over 2-3 d after hatching (Faber et al. 2002). ASH neuronal death was monitored by observing loss of GFP expression in neurons (FIG. 29A). At day 1 (day of hatching), 100% of the ASH neurons are alive, but at 3 days, only 30% are alive (FIG. 29C).

Since the effective concentration of compounds in C. elegans can vary widely from those in mammalian cell culture, the optimal concentrations to test in C. elegans were determined based on a novel assay. This assay was designed to determine compound concentrations that affect C. elegans physiology (Supplementary methods and Supplementary FIG. 1). All compounds were tested for rescue of ASH neuronal cell death at 2 to 3 concentrations; these concentrations ranged from those that affected C. elegans physiology to lower doses without any effects. It was found that trichostatin A (TSA), an HDAC inhibitor that rescues neurodegeneration in fly and mouse HD models, rescued ASH cell death in this model of HD (FIG. 29D). This result validated the assay for the discovery of small molecule therapeutics of HD. One novel compound designated revertin-2 (rev-2) was found (FIG. 27A) that rescued ASH neuronal cell death worm HD model (FIGS. 29B and D.) This compound was protective in all three mutant htt versions and the PC12 model (FIGS. 28A-C). Thus, the ST14A cell culture model can be used to discover compounds that are active in in vivo models of HD. In the screen, a series of structurally related compounds (FIGS. 20 and 27A.) was identifed, designated as revertin-1 series (rev-1a, 1b, 1c) that selectively rescued cell death in all mutant htt-expressing cell lines (FIG. 27C). Compounds in this series were active in both the worm and PC12 HD models (FIGS. 27B, D, and E). The revertin-1 series has certain features making it an attractive candidate for lead development including different analogues that are active, a low molecular weight (˜400) making it likely that it can cross the blood brain barrier; furthermore, it is synthetically tractable.

Novel Compounds Rescue Toxicity in Multiple HD Models.

Based on the results from testing in diverse HD models, two novel compounds (FIG. 27A), named revertin-1a (rev-1a) and revertin-2 (rev-2) were active in multiple HD models. rev-1a was one of a series of structurally related compounds (rev-1a, 1b, 1c) (FIG. 20). rev-1a and rev-2 rescued cell death independent of htt context. The rescue of cell death was confirmed on morphological criteria (FIG. 27B). Dose-response experiments of selective death rescue for rev-1a in all mutant htt-expressing cell lines and ST14A cells is shown in FIG. 27C. A dose response for Trypan blue exclusion based viability assay is shown for both compounds (FIG. 27D). These compounds did not affect mutant-htt or T-ag expression (FIG. 27E). Since caspase-dependent pathways are implicated in cell death in both ST14A and N548 mutant cells (FIG. 23A and 23B) (Rigamonti, D., et al., 2000, J Neurosci 20(10): 3705-3713), one explanation for selective cell death rescue by these compounds might be differential inhibition of caspase activation in the two cell lines. Therefore, these compounds were tested for suppression of caspase activation by monitoring accumulation of the activated cleaved forms of caspase-3 and caspase-7, by western blotting. These caspases are downstream effectors of cell death that are activated within 4 to 8 h after serum deprivation and stay active beyond 24h. N548 mutant and ST14A cells were incubated in serum at 33° C. or serum deprived media (SDM) with rev-2 or rev-1a treatment for 20 h at 39° C. Neither cell line displayed caspase-3 or caspase-7 cleaved fragments when cultured in serum-containing media (FIG. 27F). However in SDM at 39° C., they showed similar levels of caspase-3 and caspase-7 cleaved fragments. Rev-2 and rev-1a showed a dose dependent and selective inhibition of caspase-3 activation in N548 mutant cells, but not in ST14A cells. These compounds did not substantially inhibit caspase-7 activation in either cell line.

Both rev-1a and rev-2 also rescued cell death in the PC12 model based on morphological criteria and alamar blue reduction (FIG. 28A-C). A dose-response experiment for rescue of PC12 viability by rev-2 using the Trypan blue exclusion assay demonstrated a maximal rescue of ˜40% compared to BOC-D-fmk (FIG. 38C). Rev-1a did not show a clear rescue based on the Trypan blue exclusion assay. The reasons for this discrepancy in the results from the alamar blue reduction and trypan blue dye exclusion assays for rev-la are not clear—it may be due to different aspects of a cell's viability being measured by each assay or differences in sensitivities of these assays. The latter idea is supported by the fact that BOC-D-fmk rescues viability completely based on alamar blue, but only ˜50% based on Trypan blue exclusion.

As described earlier, rev-1a and rev-2 rescued neuronal death in the C. elegans model, as assayed by GFP expression in ASH neurons to an extent comparable to the HDAC inhibitor, trichostatin A (FIG. 29D). Both rev-1a (but not rev-1b or 1c) and rev-2 showed rescue at the highest soluble concentration of these compounds. Structures and activity of 7 active and 7 informative inactive analogs of R-1a are provided in FIG. 36. Also tested were 23 structural analogs of R-2 and found two active analogs (FIG. 37). The analysis of SAR for R-1a and R-2 is presented below. These compounds are effective at low micromolar concentrations and would likely require optimization to enhance potency. This structure activity information confirms the compound specificity and will be useful for synthesis of compounds with increased potency and efficacy.

Compounds of rev-1 series have features making them attractive candidates for lead development, including a number of active analogues that are synthetically tractable and a low molecular weight (˜400 dalton) making it likely they will cross the blood-brain barrier. In summary, these results demonstrate optimization of a striatal HTS model for HD and its use to discover novel lead-like compounds that are active in multiple models.

Structure Activity Relationship (SAR) for R-1a.

By analyzing the structural analogs of la that were tested in the primary screen (all at 4 μg/mL), some features of the R-1a scaffold required for its activity (FIG. 36) were discerned. Fluorination of both phenyl rings appears to increase activity, as replacement of the fluoro substituent with even a bromo substituent resulted in a loss of activity. Changing the substitution of the benzoyl moiety from monofluoro to dichloro resulted in an increase in activity, even in the absence of a fluoro substituent on the N-phenyl ring.

SAR for R-2.

23 structural analogs of R-2 (FIG. 37) were purchased and tested. Two of the analogs were active with similar efficacy to R-2, resulting in a limited degree of information on the structure-activity relationship. For example, it was found that the tricyclic structure of R-2 alone was insufficient to recapitulate its activity, without the alpha-beta-unsaturated ketone substituent on the piperidine ring. This double bond (conjugated to the carbonyl of this substituent) appears crucial for the activity of R2, as all analogs lacking this feature were inactive. Of interest, different bulky groups can be placed off of this double bond and activity is maintained. This information may be useful in improving potency, activity, solubility and pharmacokinetic parameters for in vivo delivery.

Compounds of R-1 Series are Active in a Brain Slice-Based HD Assay.

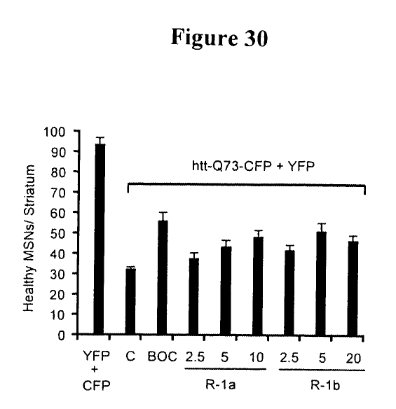

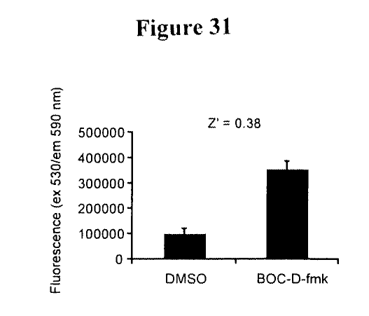

In order to assess R1-a and R-2 efficacy in a more in vivo like HD model, these compounds were tested in a previously described brain slice-based HD assay. (Khoshnan, A. et al. Activation of the IkappaB kinase complex and nuclear factor-kappaB contributes to mutant huntingtin neurotoxicity. The Journal of Neuroscience, 24, 7999-8008 (2004)). In this model, rat brain slices (postnatal day 10) are co-transfected with expression vectors for human htt exon-1 containing 73 glutamines as a cyan fluourescent fusion protein (htt-Q73-CFP) and a yellow fluorescent protein (YFP) reporter to monitor morphology of transfected neurons. Degeneration in medium spiny neurons (MSNs), a group of striatal neurons most affected in HD, is induced over 4-7 days in htt-Q73-CFP expressing cells but not in CFP transfected cells (FIG. 30). MSN health is assayed by observing morphology and integrity of transfected MSNs at day 5. BOC-D-fmk rescued MSN degeneration in this model suggesting that caspases have a role in this HD model as well (FIG. 30). Both R-1a and R-1b rescued MSN degeneration in a dose dependent manner (FIG. 30), while R1-c and R-2 did not show significant rescue. Importantly, R1-a and R1-b showed activity in this model at ˜5μg/ml which was comparable to their EC50 in the ST14A model (4 μg/ml), suggesting a similar mechanism of action in this model.

Medicinal Chemistry Based Filtering and Toxicity Testing.

In order to prioritize compounds that are more likely to be drug candidates, all novel compounds were subjected to in silico medicinal chemistry filtering. A Lipinski's rules based filter that is widely used to predict oral bioavailability for potential drug leads was used.

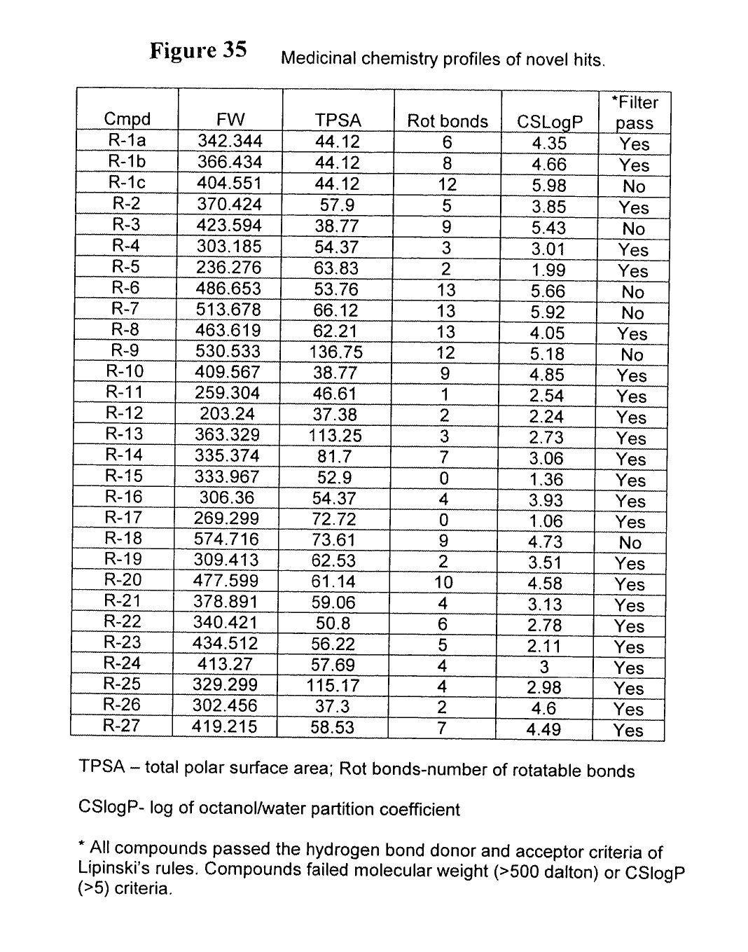

Molecular descriptors were calculated using a commercially available prediction program (www.chemsilico.com) (FIG. 35). 23 of the 29 selective novel compounds including R-1a and R-2 passed Lipinski's filters suggesting adequate oral bioavailability (FIG. 33). The therapeutic window for all compounds was reviewed by assessing toxicity in N548-mutant cells. 17 of all 46 compounds were toxic at higher concentrations while 29 showed no toxicity at the highest concentration tested (FIG. 33 and FIG. 24).

4.3 Discussion

A central feature of HD pathology is neuronal loss in the striatum and cortex that results in a fatal outcome. Moreover, there is no therapy for HD. Potential therapeutic agents on the horizon exhibit a mild amelioration of the disease phenotype in animal models, underscoring the need for more efficacious therapeutic agents. Few novel compounds based on aggregation screens and cell culture based HD models have been identified (Zhang, X., et al., 2005, Proc Natl Acad Sci USA 102(3): 892-897).

A number of cell culture and in vivo HD models that show enhanced cell death have been developed (Sipione, S., and Cattaneo, E., 2001, Mol Neurobiol 23(1): 21-51). In an effort to find therapeutic agents and to illuminate mechanisms of mutant htt toxicity, a screen using a striatal cell culture model of HD was conducted. In this model, perturbation of cellular pathways by mutant htt enhances susceptibility to cell death by serum deprivation (Rigamonti, D., et al., 2000, J Neurosci 20(10): 3705-3713). This striatal neuronal cell viability assay was optimized for HTS in a 384 well format. It was a simple assay performed over a short duration (3 d) and achieved a throughput of ˜5,000 compounds in a single run, paving the way for larger screens. 47,000 compounds were screened and identified 46 compounds that inhibited cell death in this model, based on three distinct cell viability criteria.

A major challenge in drug discovery is prioritizing a large number of hits identified in HTS. A strategy of systematically testing all 46 compounds in 3 different HD models was carried out, and found a few hits that were active in multiple HD models; for example rev-1 and rev-2 were active in 3 distinct HD models (ST14A, PC12 and C.elegans). This testing revealed a low overlap of activity across different models. There may be numerous reasons for this result, including a lack of conserved targets in different organisms or cell types (striatal neurons compared to PC12 cells of neuroendocrine origin), different htt protein contexts (exon 1 in PC12 and yeast; N171 in C. elegans model), levels of transgene expression (yeast and PC12 have relatively high expression) and permeability differences (yeast and C. elegans). This poor overlap is not surprising since even for the same assay, using different assay detection technologies can result in the identification of non-overlapping, but functionally relevant hits (Wu, X., et al., 2003, Journal of Biomolecular Screening 8(4): 381-392). Thus, inactivity of a compound in the subset of secondary HD models tested does not imply a lack of relevance for HD. Further testing these compounds in a wider set of HD assays is likely to identify additional hits relevant to HD.

Another issue addressed was to identify small molecules that selectively target disease pathways. Most HD cell viability screens do not discriminate between compounds that specifically target mutant htt-perturbed cell death pathways and general cell death pathways, (Aiken, C. T., et al., 2004, Neurobiol Dis 16(3): 546-555). In this assay, serum deprivation of ST14A and N548 mutant-htt expressing cell lines causes caspase dependent death that is suppressed non-selectively by the pan-caspase inhibitor, BOC-D-fmk (FIG. 23A and 23B). By testing compounds for cell death suppression in ST14A cells, dozens of compounds were identified that selectively inhibited cell death in N548 mutant cells. These results suggest that there are differences in the cell death pathways between wild type and mutant htt neurons and that small molecules can target these pathways. This approach is similar to synthetic lethal screening in cancer models where the aim is to selectively kill tumor cells with specific genetic elements (Dolma, S., et al., 2003, Cancer Cell 3(3): 285-296). These results demonstrate that though the phenotypes are reversed (cell death vs cell survival) it is possible to detect genotype-selective agents that only affect mutant cells. Not only can these compounds be specific therapeutic agents, but they may be useful probes for identifying disease pathways and targets for therapy.

Aberrant caspase activation has been implicated in HD. Enhanced and aberrant caspase-3 processing in N548 mutant cells has been reported (Rigamonti, D., et al., 2000, J Neurosci 20(10): 3705-3713). Aberrant activation of caspase-8 has also been reported in HD. Pharmacological and genetic inhibition of caspase-1(Ona, V. O., et al., 1999, Nature 399(6733): 263-267) and caspase-3 activation (Chen, M., et al., 2000, Nature Medicine 6(7): 797-801; Hersch, S., et al. 2003, Ann Neurol 54(6): 841; author reply 842-843; Wang, X., et al., 2003, Proc Natl Acad Sci USA 100(18): 10483-10487) have been shown to delay disease onset and extend life span in transgenic mice HD model. It was found that selective suppression of death by rev-1 and rev-2 in N548 mutant cells correlates with selective inhibition of caspase-3 activation in N548 mutant cells. These results suggest that there are differences in pathways that cause caspase-3 activation in N548 mutant cells compared to ST14A cells. Selective inhibition of caspase activation by these compounds could be a useful therapeutic intervention.

As a step in defining the mechanisms of these selective compounds, they were grouped on the basis of their selective rescue in cell lines expressing htt-transgenes of different length (FIG. 25). This strategy can help elucidate the mechanism of other compounds in the same class as demonstrated by this discovery of novel microtubule inhibitors. Further, all compounds active in PC12 (exon 1 htt) were from htt length independent (class I) and N63 and N548 selective (class IIB) classes, supporting the idea that htt length-based classification of compounds has underlying mechanistic basis.

Multiple mechanisms have been proposed to contribute to HD pathology (Ross, C. A., 2002, Neuron 35(5): 819-822; Rangone, H., et al. 2004, Pathol Biol (Paris) 52(6): 338-342). Several studies have suggested that htt context modifies polyQ toxicity, since gene-expression changes, disease severity and subcellular distribution of mutant htt are modified by htt protein context. For example, mice expressing exon 1 of htt (R6/2) show a faster disease progression in comparison to transgenic mice with full-length mutant htt. Also protein context may play a role in the selectivity of the brain regions affected in HD, since other polyQ expansion disorders in distinct proteins affect different brain regions. It was reasoned that if polyQ triggers toxicity by common pathways in each htt context, then all compounds should be active in a context-independent manner. This identification of compounds that prevent htt toxicity in a context-dependent manner and supports a model in which mutant htt causes toxicity by affecting multiple cellular pathways, some of these pathways are common to different htt contexts while others are unique to each htt context.

Since both full length and smaller htt fragments are reported in HD patient brains; these results also raise questions about the htt fragment length used in HD models for assessing drug efficacy. Various HD models use different lengths of htt protein, ranging from small N-terminal fragments to full-length htt. Most models currently used to assess potential HD therapeutics express mutant htt's exon 1 since it generally causes a more severe phenotype. These results suggest that potential therapeutic agents should be tested in models where polyQ is expressed in more than one htt protein contexts to avoid overlooking therapeutically useful compounds. Alternatively, compounds that are active in multiple contexts or are context-independent are more likely to be effective in HD and possibly other polyQ disorders and could be prioritized in trials. For example, rev-1 and rev-2 were effective in a context-independent manner and were also effective in both C. elegans and PC12 HD models.

There are at least 8 polyQ disorders in addition to HD that are caused by polyQ expansions in different proteins (Gatchel, J. R., and Zoghbi, H. Y., 2005, Nature Reviews Genetics 6(10): 743-755) and have no therapy at present (Di Prospero, N. A., and Fischbeck, K. H., 2005, Nature Reviews Genetics 6(10): 756-765). Testing this subset of compounds in models of other polyQ disorders could identify drug leads for these disorders and probes to understand common mechanisms of polyQ toxicity. Overall, the subset of small molecules presented here should accelerate the discovery of therapeutics for HD and serve as probes for dissecting the pathophysiology of HD and possibly other polyQ disorders. The strategies present here, including selectivity-profiling-based classification and systemic testing in multiple model systems are applicable to other screening efforts.

### Example 5

### Isolation of Compounds that Reduce Cell Death in the ST14A Assay

Huntington's disease (HD) is one of at least nine inherited neurological disorders caused by PolyQ, or trinucleotide (CAG) repeat, expansion (others being Kennedy's disease, dentatorubro-pallidoluysian atrophy, and six forms of spinocerebellar ataxia). One aim of these experiments is to identify small molecule suppressors of PolyQ neurotoxicity and to elucidate mechanisms of polyQ neurotoxicity through studying the functional means by which the identified compounds suppress PolyQ-expanded Htt toxicity.

As described above in Example 4, the ST14A cell assay may be useful for identifying compounds that reduce cellular lethality due to PolyQ expansion. In addition to the molecules described in the preceding examples, use of the ST14A screening assay revealed compounds that suppress PolyQ-expanded Htt toxicity. Three classes of compounds were identified in the screen: 141, 178, and 180. These compounds, along with a selection of their analogs identified in the screen, are listed in FIG. 19.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A sterile pharmaceutical composition comprising a compound having a formula selected from the group consisting of Formula I and Formula II, in an amount effective in inhibiting neuronal cell death: 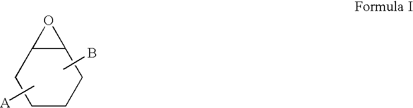 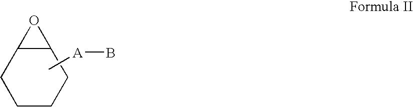
where A is a substituted or unsubstituted cycloalkyl, aryl, or heterocyclyl;
where B is a substituted tetrahydrofuran-2-one: 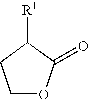
and where R¹ is an alkylheterocyclyl group having the formula (CH₂)ₙ-D, where n=1-3 and D is selected from the group consisting of substituted or unsubstituted quinoline; substituted or unsubstituted isoquinoline; substituted or unsubstituted piperidine; substituted or unsubstituted piperazine; and substituted or unsubstituted pyrrolidine.

2. The composition of claim 1, which further comprises a pharmaceutical carrier.

3. A sterile pharmaceutical composition comprising a compound having Formula III in an amount effective in inhibiting neuronal cell death: 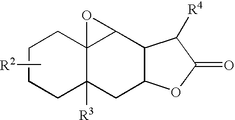
where R² is selected from the group consisting of H, methyl, alkyl,and cycloalkyl;
where R³ is selected from the group consisting of H, methyl, alkyl, and cycloalkyl;
where R⁴ is (CH₂)ₘ-E, where m=1 -3 and E is selected from the group consisting of substituted or unsubstituted quinoline; substituted or unsubstituted isoquinoline, substituted or unsubstituted piperidine; substituted or unsubstituted piperazine; and substituted or unsubstituted pyrrolidine.

4. The composition of claim 3, further comprising a pharmaceutical carrier.

5. A sterile pharmaceutical composition comprising a compound having Formula IV in an amount effective in inhibiting neuronal cell death: 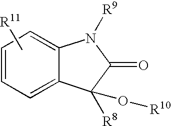
where R¹ may be absent (in which case the bond to oxygen is a double bond) or is selected from the group consisting of H, (C₁₋₄)alkyl, (C₁₋₄)alkenyl, (C₁₋₄)alkylcarbonyl(C₁₋₄)alkyl, (C₁₋₄)alkenylcarbonyl(C₁₋₄)alkyl, (C₁₋₄)alkylcarbonyl(C₁₋₄)alkenyl, (C₁₋₄)alkylaryl, and (C₁₋₄)alkylcarbonyl(C₁₋₄)alkenylary;
where R⁹ is selected from the group consisting of H and (C₁₋₄)alkyl;
where R¹⁰ may be absent or is selected from the group consisting of H, (C₁₋₄)alkyl, (C₁₋₄)alkylcarbonyl(C₁₋₄)alkyl, (C₁₋₄)alkylhydroxyl, and (C₁₋₄)alkylhydroxyl(C₁₋₄)alkyl; and
where R¹¹ is H or (C₁₋₄)alkyl.

6. The composition of claim 5, further comprising a pharmaceutical carrier.

7. A sterile pharmaceutical composition comprising a compound having Formula V in an amount effective in inhibiting neuronal cell death: 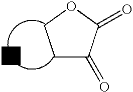
wherein F comprises one or two ring structures containing 10 carbon atoms.

8. The composition of claim 7, further comprising a pharmaceutical carrier.

9. A sterile pharmaceutical composition comprising a compound having Formula VI in an amount effective in inhibiting neuronal cell death: 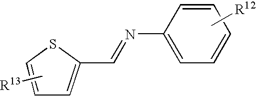
wherein R¹² is selected from the group consisting of H, (C₁₋₄)alkyl, amide, (C₁₋₄)alkylamide; (C₁₋₄)alkylcarbonyl, (C₁₋₄)alkoxycarbonyl, and sulfonyl; and
wherein R¹³ is selected from the group consisting of H, (C₁₋₄)alkyl, NO₂, (C₁₋₄)alkylcarbonyl, (C₁₋₄)alkoxycarbonyl and sulfonyl.

10. The composition of claim 9, further comprising a pharmaceutical carrier.

11. A sterile pharmaceutical composition comprising a compound having Formula VII in an amount effective in inhibiting neuronal cell death: 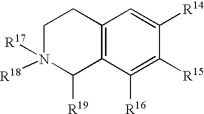
where R¹⁴ and R¹⁵ may be the same or different, and are selected from the group consisting of H, (C₁₋₄)alkyl, oxy, (C₁₋₄)alkoxy, and (C₁₋₄)alkoxycarbonyl;
where R¹⁶ is selected from the group consisting of H, (C₁₋₄)alkyl, (C₁₋₄)alkoxy, (C₁₋₄)alkoxyalkyl, and (C₁₋₄)alkoxycarbonyl;
where R¹⁷ is selected from the group consisting of H or (C₁₋₄)alkyl;
where R¹⁸ may be absent or is selected from the group consisting of H and methyl; and
where R¹⁹ is selected from the group consisting of (C₁₋₄)alkylcarbonyl(C₁₋₄)alkyl, (C₁₋₄)alkylcarbonylaryl, (C₁₋₄)alkylcarbonyl(C₁₋₄)alkenylaryl, amido(C₁₋₄)alkylaryl and amido(C₁₋₄)alkylheteroaryl.

12. The composition of claim 11 where R¹⁹ is (CH₂)ₒcarbonyl-G-J, where o=1-3, G is selected from the group consisting of (C₁₋₄)alkyl, (C₁₋₄)alkenyl, and (C₁₋₄)alkylhydroxy, and J is selected from the group consisting of substituted or unsubstituted aryl, substituted or unsubstituted heteroaryl, substituted or unsubstituted phenyl, substituted or unsubstituted pyridine, substituted or unsubstituted pyrazine, substituted or unsubstituted pyrimidine, substituted or unsubstituted pyrrole, substituted or unsubstituted furan, substituted or unsubstituted isoxazole, and substituted or unsubstituted isothiazole.

13. The composition of claim 11, further comprising a pharmaceutical carrier.

14. The composition of claim 12, further comprising a pharmaceutical carrier.

15. A sterile pharmaceutical composition comprising a compound having Formula VIII in an amount effective in inhibiting neuronal cell death: 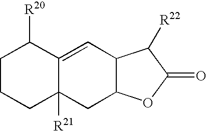
where R²⁰ is selected from the group consisting of H, (C₁₋₄)alkyl, and (C₁₋₄)alkoxy;
wherein R²¹ is selected from the group consisting of H, (C₁₋₄)alkyl, and (C₁₋₄)alkoxy;
wherein R²² is selected from the group consisting of H, (C₁₋₄)alkyl, (C₁₋₄)alkoxy, (C₁₋₄)alkyl(4-7)cyclo(C₁₋₄)alkyl, (C₁₋₄)alkylaryl, (C₁₋₄)alkylheteroaryl, and (C₁₋₄)alkylhetero(4-7)cyclyl.

16. The composition of claim 15 wherein R²² is an alkylheterocyclyl where the heterocyclic group is selected from the group consisting of a substituted or unsubstituted piperidine, a substituted or unsubstituted piperazine, a substituted or unsubstituted pyrrolidine, a substituted or unsubstituted phenyl, a sustituted or unsubstituted pyrazine, a substituted or unsubstituted pyridine, a substituted or unsubstituted pyrimidine, a substituted or unsubstituted pyrrole and a substituted and unsubstituted furan.

17. The composition of claim 15, further comprising a pharmaceutical carrier.

18. The composition of claim 16, further comprising a pharmaceutical carrier.

19. A sterile pharmaceutical composition comprising a compound having Formula IX in an amount effective in inhibiting neuronal cell death: 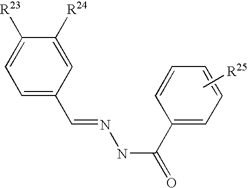
wherein R²³ and R²⁴ may be the same or different and are selected from the group consisting of H, (C₁₋₄)alkyl, hydroxy, and (C₁₋₄)alkoxy;
wherein R²⁵ is selected from the group consisting of H, (C₁₋₄)alkyl, (C₁₋₄)alkoxy, hydroxy, and halogen.

20. The composition of claim 19, further comprising a pharmaceutical carrier.

21. A sterile pharmaceutical composition comprising a compound having Formula X in an amount effective in inhibiting neuronal cell death: 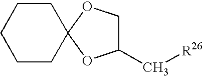
wherein R²⁶ is a heterocyclyl group of 4, 5, 6, or 7 members comprising N.

22. The composition of claim 21, where R²⁶ is a heterocyclyl comprising O in an epoxide linkage, and substituted with R²⁷, where R²⁷ is selected from the group consisting of (C₁₋₄)alkyl or aryl.

23. The composition of claim 21, further comprising a pharmaceutical carrier.

24. The composition of claim 22, further comprising a pharmaceutical carrier.

25. A sterile pharmaceutical composition comprising a compound having Formula XI in an amount effective in inhibiting neuronal cell death: 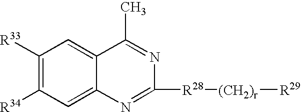
wherein r is 0, 1, or 2;
wherein R²⁸ is selected from the group consisting of NH and S;
wherein R²⁹ is selected from the group consisting of hydroxy, a 4-7 member heterocyclyl group, and carbamidoyl (—C(═NH)—NH2);
wherein R³³ and R³⁴ are selected from the group consisting of H and (C₁₋₄)alkyl.

26. The composition of claim 25, where R²⁹ is a 4-7 member substituted heterocyclyl group where the heteroatom is N, and where the substitutions are R³⁰, R³¹ , and R³²;
wherein R³⁰, R³¹, and R³² are selected from the group consisting of H, hydroxy, and (C₁₋₄)alkyl.

27. The composition of claim 25, where R²⁹ is a substituted carbamidoyl group (—C(═N¹H)—N²H2) where the substitution may be on N¹ or ² and where the substitution is selected from the group consisting of (C₁₋₄)alkyl, aryl, or C₁₋₄ alkylaryl.

28. The composition of claim 25, further comprising a pharmaceutical carrier.

29. The composition of claim 26, further comprising a pharmaceutical carrier.

30. The composition of claim 27, further comprising a pharmaceutical carrier.

31. A sterile pharmaceutical composition comprising a compound having Formula XII in an amount effective in inhibiting neuronal cell death: 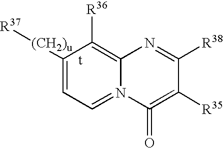
wherein R³⁵ is selected from the group consisting of H, (C₁₋₄)alkyl, I, F, and Br;
wherein R³⁶ is selected from the group consisting of hydroxy and keto;
wherein t is a single bond or a double bond;
wherein u is 0 or 1;
wherein R³⁷ is selected from the group consisting of Br, F, I, and a 4-7 member heterocyclyl group.

32. The composition of claim 31, where R³⁷ is a 4-7 member heterocyclyl group substituted with (C₁₋₄)alkyl.

33. The composition of claim 31, further comprising a pharmaceutical carrier.

34. The composition of claim 32, further comprising a pharmaceutical carrier.

35. A method of inhibiting neuronal cell death comprising administering, to a neuronal cell, an effective amount of a compound having a formula selected from the group consisting of Formula I and Formula II: 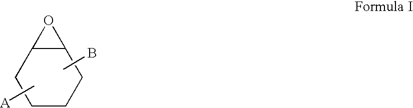 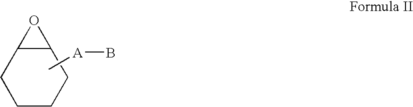
where A is a substituted or unsubstituted cycloalkyl, aryl, or heterocyclyl;
where B is a substituted tetrahydrofuran-2-one: 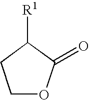
and where R¹ is an alkylheterocyclyl group having the formula (CH₂)ₙ-D, where n=1-3 and D is selected from the group consisting of substituted or unsubstituted quinoline; substituted or unsubstituted isoquinoline; substituted or unsubstituted piperidine; substituted or unsubstituted piperazine; and substituted or unsubstituted pyrrolidine.

36. A method of inhibiting neuronal cell death comprising administering, to a neuronal cell, an effective amount of a compound having Formula III: 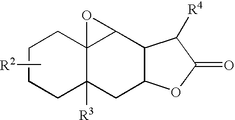
where R² is selected from the group consisting of H, methyl, alkyl,and cycloalkyl;
where R³ is selected from the group consisting of H, methyl, alkyl, and cycloalkyl;
where R⁴ is (CH₂)ₘ-E, where m=1-3 and E is selected from the group consisting of substituted or unsubstituted quinoline; substituted or unsubstituted isoquinoline, substituted or unsubstituted piperidine; substituted or unsubstituted piperazine; and substituted or unsubstituted pyrrolidine.

37. A method of inhibiting neuronal cell death comprising administering, to a neuronal cell, an effective amount of a compound having Formula IV: 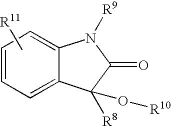
where R⁸ may be absent (in which case the bond to oxygen is a double bond) or is selected from the group consisting of H, (C₁₋₄)alkyl, (C₁₋₄)alkenyl, (C₁₋₄)alkylcarbonyl(C₁₋₄)alkyl, (C₁₋₄)alkenylcarbonyl(C₁₋₄)alkyl, (C₁₋₄)alkylcarbonyl(C₁₋₄)alkenyl, (C₁₋₄)alkylaryl, and (C₁₋₄)alkylcarbonyl(C₁₋₄)alkenylary;
where R⁹ is selected from the group consisting of H and (C₁₋₄)alkyl;
where R¹⁰ may be absent or is selected from the group consisting of H, (C₁₋₄)alkyl, (C₁₋₄)alkylcarbonyl(C₁₋₄)alkyl, (C₁₋₄)alkylhydroxyl, and (C₁₋₄)alkylhydroxyl(C₁₋₄)alkyl; and
where R¹¹ is H or (C₁₋₄)alkyl.

38. A method of inhibiting neuronal cell death comprising administering, to a neuronal cell, an effective amount of a compound having Formula V: 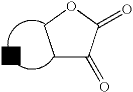
wherein F comprises one or two ring structures containing 10 carbon atoms.

39. method of inhibiting neuronal cell death comprising administering, to a neuronal cell, an effective amount of a compound having Formula VI: 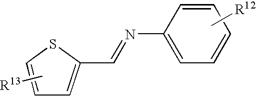
wherein R¹² is selected from the group consisting of H, (C₁₋₄)alkyl, amide, (C₁₋₄)alkylamide; (C₁₄)alkylcarbonyl, (C₁₋₄)alkoxycarbonyl, and sulfonyl; and
wherein R¹³ is selected from the group consisting of H, (C₁₋₄)alkyl, NO₂, (C₁₋₄)alkylcarbonyl, (C₁₋₄)alkoxycarbonyl and sulfonyl.

40. A method of inhibiting neuronal cell death comprising administering, to a neuronal cell, an effective amount of a compound having Formula VII: 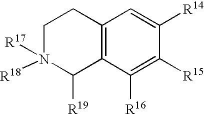
where R¹⁴ and R¹⁵ may be the same or different, and are selected from the group consisting of H, (C₁₋₄)alkyl, oxy, (C₁₋₄)alkoxy, and (C₁₋₄)alkoxycarbonyl;
where R¹⁶ is selected from the group consisting of H, (C₁₋₄)alkyl, (C₁₋₄)alkoxy, (C₁₋₄)alkoxyalkyl, and (C₁₋₄)alkoxycarbonyl;
where R¹⁷ is selected from the group consisting of H or (C₁₋₄)alkyl;
where R¹⁸ may be absent or is selected from the group consisting of H and methyl; and
where R¹⁹ is selected from the group consisting of(C₁₋₄)alkylcarbonyl(C₁₋₄)alkyl, (C₁₋₄)alkylcarbonylaryl, (C₁₋₄)alkylcarbonyl(C₁₋₄)alkenylaryl, amido(C₁₋₄)alkylaryl and amido(C₁₋₄)alkylheteroaryl.

41. The method of claim 40 where R¹⁹ is (CH₂)ₒcarbonyl-G-J, where o=1-3, G is selected from the group consisting of(C₁₋₄)alkyl, (C₁₋₄)alkenyl, and (C₁₋₄)alkylhydroxy, and J is selected from the group consisting of substituted or unsubstituted aryl, substituted or unsubstituted heteroaryl, substituted or unsubstituted phenyl, substituted or unsubstituted pyridine, substituted or unsubstituted pyrazine, substituted or unsubstituted pyrimidine, substituted or unsubstituted pyrrole, substituted or unsubstituted furan, substituted or unsubstituted isoxazole, and substituted or unsubstituted isothiazole.

42. A method of inhibiting neuronal cell death comprising administering, to a neuronal cell, an effective amount of a compound having Formula VIII: 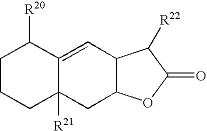
where R²⁰ is selected from the group consisting of H, (C₁₋₄)alkyl, and (C₁₋₄)alkoxy;
wherein R²¹ is selected from the group consisting of H, (C₁₋₄)alkyl, and (C₁₋₄)alkoxy;
wherein R²² is selected from the group consisting of H, (C₁₋₄)alkyl, (C₁₋₄)alkoxy, (C₁₋₄)alkyl(4-7)cyclo(C₁₋₄)alkyl, (C₁₋₄)alkylaryl, (C₁₋₄)alkylheteroaryl, and (C₁₋₄)alkylhetero(4-7)cyclyl.

43. The method of claim 42 wherein R²² is an alkylheterocyclyl where the heterocyclic group is selected from the group consisting of a substituted or unsubstituted piperidine, a substituted or unsubstituted piperazine, a substituted or unsubstituted pyrrolidine, a substituted or unsubstituted phenyl, a sustituted or unsubstituted pyrazine, a substituted or unsubstituted pyridine, a substituted or unsubstituted pyrimidine, a substituted or unsubstituted pyrrole and a substituted and unsubstituted furan.

44. A method of inhibiting neuronal cell death comprising administering, to a neuronal cell, an effective amount of a compound having Formula IX: 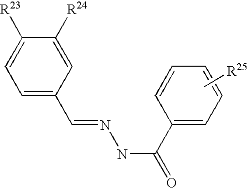
wherein R²³ and R²⁴ may be the same or different and are selected from the group consisting of H, (C₁₋₄)alkyl, hydroxy, and (C₁₋₄)alkoxy;
wherein R²⁵ is selected from the group consisting of H, (C₁₋₄)alkyl, (C₁₋₄)alkoxy, hydroxy, and halogen.

45. A method of inhibiting neuronal cell death comprising administering, to a neuronal cell, an effective amount of a compound having Formula X: 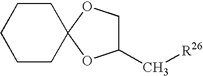
wherein R²⁶ is a heterocyclyl group of 4, 5, 6, or 7 members comprising N.

46. The method of claim 45, where R²⁶ is a heterocyclyl comprising O in an epoxide linkage, and substituted with R²⁷, where R²⁷ is selected from the group consisting of (C₁₋₄)alkyl or aryl.

47. A method of inhibiting neuronal cell death comprising administering, to a neuronal cell, an effective amount of a compound having Formula XI: 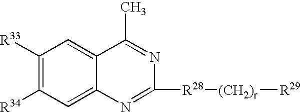
wherein r is 0, 1, or 2;
wherein R²⁸ is selected from the group consisting of NH and S;
wherein R²⁹ is selected from the group consisting of hydroxy, a 4-7 member heterocyclyl group, and carbamidoyl (—C(═NH)—NH2);
wherein R³³ and R³⁴ are selected from the group consisting of H and (C₁₋₄)alkyl.

48. The method of claim 47, where R²⁹ is a 4-7 member substituted heterocyclyl group where the heteroatom is N, and where the substitutions are R³⁰, R³¹, and R³²;
wherein R³⁰, R³¹, and R³² are selected from the group consisting of H, hydroxy, and (C₁₋₄)alkyl.

49. The method of claim 47, where R²⁹ is a substituted carbamidoyl group (—C(═N¹H)—N²H2) where the substitution may be on N¹ or ² and where the substitution is selected from the group consisting of (C₁₋₄) alkyl, aryl, or C₁₋₄ alkylaryl.

50. A method of inhibiting neuronal cell death comprising administering, to a neuronal cell, an effective amount of a compound having Formula XII: 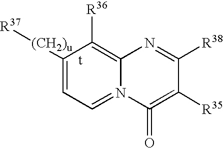
wherein R³⁵ is selected from the group consisting of H, (C₁₋₄)alkyl, I, F, and Br;
wherein R³⁶ is selected from the group consisting of hydroxy and keto;
wherein t is a single bond or a double bond;
wherein u is 0 or 1;
wherein R³⁷ is selected from the group consisting of Br, F, I, and a 4-7 member heterocyclyl group.

51. The method of claim 50, where R³⁷ is a 4-7 member heterocyclyl group substituted with (C₁₋₄)alkyl.

52. A sterile pharmaceutical composition comprising a compound having Formula XIII in an amount effective in inhibiting neuronal cell death: 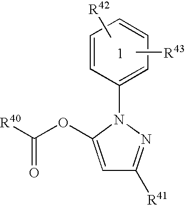
where R⁴⁰ may be may be a substituted or unsubstituted aromatic, substituted or unsubstituted diaromatic, or C₁-C₁₀ alkyl; wherein substituent groups may be H, halogen, C₁-C₄ alkyl groups, or alkoxy groups; and wherein the number of substituents may be one, two, or more than two;
R⁴¹ may be a (C₁₋₄)alkyl, alkoxy, aromatic ring, or dimethyl group;
Ring 1 may be additionally substituted, wherein R⁴² and R⁴³ may be H or a halogen, preferably flourine or chlorine; and
R⁴² and R⁴³ may be the same or may be different substituent groups.

53. The composition of claim 52 further comprising a pharmaceutical carrier.

54. A sterile pharmaceutical composition comprising a compound having Formula XIV in an amount effective in inhibiting neuronal cell death: 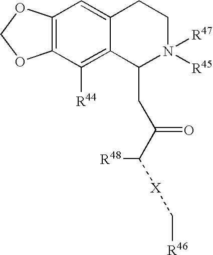
wherein R⁴⁴ and R⁴⁵ may be C₁-C₄ alkoxy groups;
“—X—” may be a single or double bond or an amide bond.
R⁴⁶ may be absent or, for example, one of the following substituent groups: 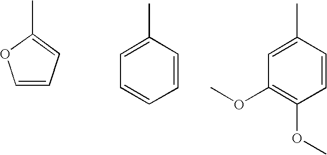
or may be a substituted aromatic rin or heterocyclic (optionally aromatic ring);
R⁴⁷ may be absent or may be hydrogen or methyl (in which case the N is a quaternary ammonium ion); and
R⁴⁸may be C or N or O.

55. The composition of claim 54 further comprising a pharmaceutical carrier.

56. A method of inhibiting neuronal cell death comprising administering, to a neuronal cell, an effective amount of a compound having Formula XIII: 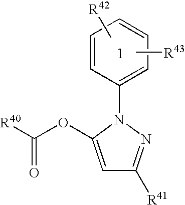
where R⁴⁰ may be may be a substituted or unsubstituted aromatic, substituted or unsubstituted diaromatic, or C₁-C₁₀ alkyl; wherein substituent groups may be H, halogen, C₁-C₄ alkyl groups, or alkoxy groups; and wherein the number of substituents may be one, two, or more than two;
R⁴¹ may be a (C₁₋₄)alkyl, alkoxy, aromatic ring, or dimethyl group;
Ring 1 may be additionally substituted, wherein R⁴² and R⁴³ may be H or a halogen, preferably flourine or chlorine; and
R⁴² and R⁴³ may be the same or may be different substituent groups.

57. A method of inhibiting neuronal cell death comprising administering, to a neuronal cell, an effective amount of a compound having Formula XIV: 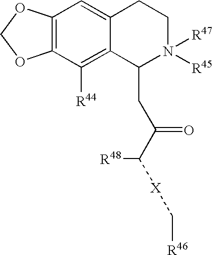
wherein R⁴⁴ and R⁴⁵ may be C₁-C₄ alkoxy groups;
“—X—” may be a single or double bond or an amide bond.
R⁴⁶ may be absent or, for example, one of the following substituent groups: 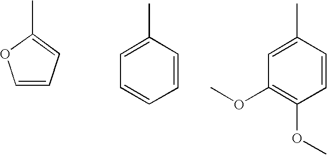
or may be a substituted aromatic ring or heterocyclic (optionally aromatic ring);
R⁴⁷ may be absent or may be hydrogen or methyl (in which case the N is a quaternary ammonium ion); and
R⁴⁸ may be C or N or O.
